(19) United States
(12) Patent Application Publication
(10) Pub. No.: US 2024/0238237 A1
(43) Pub. Date: Jul. 18, 2024

(54) CONJUGATED LINOLEIC ACID SUPPLEMENTATION FOR DISEASE TREATMENT

(71) Applicant: VIRGINIA TECH INTELLECTUAL PROPERTIES, INC., Blacksburg, VA (US)

(72) Inventor: Deborah J. Good, Blacksburg, VA (US)

(21) Appl. No.: 18/289,409

(22) PCT Filed: May 4, 2022
(86) PCT No.: PCT/US2022/027697

§ 371 Date: Nov. 3, 2023

Related U.S. Application Data
(60) Provisional application No. 63/184,001, filed on May 4, 2021.
(60) Provisional application No. 63/184,029, filed on May 4, 2021.

(51) Int. Cl. A61K 31/201; A61P 1/14; A61P 3/04; A61P 5/24

(52) U.S. Cl. CPC A61K 31/201; A61P 1/14; A61P 3/04; A61P 5/24

(57) ABSTRACT

Described in certain example embodiments herein are methods of and compositions for treating a Snordl l6 deficiency disease or symptom thereof with conjugated linoleic acid (CLA) or a formulation thereof. In some embodiments, the Snordl l6 deficiency disease is Prader-Willi Syndrome. Described in certain example embodiments herein are methods of and compositions for treating Nhlh2 associated hypogonadism with CLA or a formulation thereof.

### CROSS-REFERENCE TO RELATED APPLICATIONS

This application claims the benefit of U.S. Provisional Application No. 63/184,001, filed on May 4, 2021, entitled “Treatment of SNORD116 Deficiency Diseases, Prader-Willi Syndrome, and Symptoms Thereof,” and U.S. Provisional Application No. 63/184,029, filed on May 4, 2021, entitled “Conjugated Linoleic Acid Treatment of Hypogonadism,” the contents of which are incorporated by reference herein in their entireties.

### TECHNICAL FIELD

The subject matter disclosed herein is generally directed to conjugated linoleic acid treatment of diseases and symptoms thereof.

### BACKGROUND

Prader-Willi Syndrome (PWS) is a genetic condition that occurs in up to 1 in 10,000 live births. Individuals with PWS show initial developmental delay, significant hypotonia/muscle weakness, and typically demonstrate some level of intellectual disability, and obesity later in childhood or adolescence. For PWS patients, there is, as yet, no cure, and few treatment options. As such, there at least a need for improved treatments of PWS and/or symptoms thereof.

Citation or identification of any document in this application is not an admission that such a document is available as prior art to the present invention.

### SUMMARY

Described in certain embodiments herein are methods of treating a Snord116 deficiency, disease, or a symptom thereof in a subject in need thereof, the method comprising administering an effective amount of conjugated linoleic acid (CLA) or a formulation thereof to the subject in need thereof, optionally wherein the effective amount is administered in one or more doses. In certain example embodiments, the Snord116 deficiency disease is Prader-Willi Syndrome (PWS).

In certain example embodiments, the effective amount of CLA is at least 1,000 mg per day, optionally about 3,000 to about 5,000 mg per day. In certain example embodiments, the effective amount of the CLA is about 3-10 g/kg body weight per day, optionally about 5 g/kg body weight per day. In certain example embodiments, the CLA or formulation thereof comprises a mixture of CLA isoforms, optionally a 50:50 mixture of trans-10, cis-12 and trans9, cis-11 CLA isomers. In certain example embodiments, the CLA or formulation thereof comprises 80-100% of a mixture of CLA isoforms, optionally a 50:50 mixture of trans-10, cis-12 and trans9, cis-11 CLA isomers.

In certain example embodiments, the effective amount of CLA decreases body weight, decreases fat mass, increases lean body mass, improves hypogonadism or a symptom thereof, improves testicular morphology, modulates the α-diversity of the gut microbiome, modulates hypothalamic gene expression of one or more genes, decreases anxiety, increase sperm maturation, increase spermatogenesis, increase sperm differentiation, decrease testicular degeneration or any combination thereof in the subject in need thereof.

In certain example embodiments, the effective amount of CLA or formulation thereof is administered daily, every other day, weekly, or monthly.

In certain example embodiments, the effective amount of CLA or formulation thereof is a liquid, solid, semi-solid, or an emulsion.

In certain example embodiments, the effective amount of CLA or formulation thereof is a dietary supplement or feed or foodstuff or beverage formulation.

In certain example embodiments, the subject in need thereof is a mammal, optionally a human.

Described in certain example embodiments herein are dietary supplements, feeds, or foodstuff or beverage formulations effective for treating a Snord116 deficiency disease or a symptom thereof in a subject in need thereof, the dietary supplement comprising an amount of conjugated linoleic acid (CLA) or a formulation thereof such that the dietary supplement delivers an effective amount of the CLA to the subject in need thereof in one or more doses, optionally 1-3 doses.

In certain example embodiments, the Snord116 deficiency disease is Prader-Willi Syndrome (PWS). In certain example embodiments, the effective amount of CLA is at least 1,000 mg per day, optionally about 3,000 to about 5,000 mg per day. In certain example embodiments, the effective amount of the CLA is about 3-10 g/kg body weight per day, optionally about 5 g/kg body weight per day. In certain example embodiments, the CLA or formulation thereof comprises a mixture of CLA isoforms, optionally a 50:50 mixture of trans-10, cis-12 and trans9, cis-11 CLA isomers. In certain example embodiments, the CLA or formulation thereof comprises 80-100% of a mixture of CLA isoforms, optionally a 50:50 mixture of trans-10, cis-12 and trans9, cis-11 CLA isomers.

In certain example embodiments, the effective amount of CLA decreases body weight, decreases fat mass, increases lean body mass, improves hypogonadism or a symptom thereof, improves testicular morphology, modulates the α-diversity of the gut microbiome, modulates hypothalamic gene expression of one or more genes, decreases anxiety, increase sperm maturation, increase spermatogenesis, increase sperm differentiation, decrease testicular degeneration or any combination thereof in the subject in need thereof.

In certain example embodiments, the dietary supplement, feed, or foodstuff or beverage formulation is a liquid, solid, semi-solid, or an emulsion.

In certain example embodiments, the dietary supplement, feed, or foodstuff or beverage formulation is adapted for daily, every other day, weekly, or monthly administration.

In certain example embodiments, the subject in need thereof is a mammal, optionally a human.

Described in certain example embodiments herein are methods of treating hypogonadism in a subject having a Nhlh2 deficiency, disease, or symptom thereof, the method comprising an effective amount of conjugated linoleic acid (CLA) or a formulation thereof to the subject in need thereof.

In certain example embodiments, the effective amount of CLA is at least 1,000 mg per day, optionally about 3,000 to about 5,000 mg per day. In certain example embodiments, the effective amount of the CLA is about 3-10 g/kg body weight per day, optionally about 5 g/kg body weight per day. In certain example embodiments, the CLA or formulation thereof comprises a mixture of CLA isoforms, optionally a 50:50 mixture of trans-10, cis-12 and trans9, cis-11 CLA isomers. In certain example embodiments, the CLA or formulation thereof comprises 80-100% of a mixture of CLA isoforms, optionally a 50:50 mixture of trans-10, cis-12 and trans9, cis-11 CLA isomers.

In certain example embodiments, the effective amount of CLA decreases body weight, decreases fat mass, increases lean body mass improves hypogonadism or a symptom thereof, improves testicular morphology, increases sperm maturation, increase spermatogenesis, increase sperm differentiation, decrease testicular degeneration or any combination thereof in the subject in need thereof.

In certain example embodiments, the effective amount of CLA or formulation thereof is administered daily, every other day, weekly, or monthly.

In certain example embodiments, the effective amount of CLA or formulation thereof is a liquid, solid, semi-solid, or an emulsion.

In certain example embodiments, the effective amount of CLA or formulation thereof is a dietary supplement or feed or foodstuff or beverage formulation.

In certain example embodiments, the subject in need thereof is a mammal, optionally a human.

Described in certain example embodiments herein are dietary supplements, feeds, or foodstuff or beverage formulations hypogonadism in a subject in need thereof having a Nhlh2 deficiency, disease, or symptom thereof in a subject in need thereof, the dietary supplement comprising an amount of conjugated linoleic acid (CLA) or a formulation thereof such that the dietary supplement delivers an effective amount of the CLA to the subject in need thereof in one or more doses, optionally 1-3 doses.

In certain example embodiments, the effective amount of CLA is at least 1,000 mg per day, optionally about 3,000 to about 5,000 mg per day.

In certain example embodiments, the effective amount of the CLA is about 3-10 g/kg body weight per day, optionally about 5 g/kg body weight per day.

In certain example embodiments, the CLA or formulation thereof comprises a mixture of CLA isoforms, optionally a 50:50 mixture of trans-10, cis-12 and trans9, cis-11 CLA isomers.

In certain example embodiments, the CLA or formulation thereof comprises 80-100% of a mixture of CLA isoforms, optionally a 50:50 mixture of trans-10, cis-12 and trans9, cis-11 CLA isomers.

In certain example embodiments, the effective amount of CLA decreases body weight, decreases fat mass, increases lean body mass, improves hypogonadism or a symptom thereof, improves testicular morphology, increases sperm maturation, increase spermatogenesis, increase sperm differentiation, decrease testicular degeneration or any combination thereof in the subject in need thereof.

In certain example embodiments, the dietary supplement, feed, or foodstuff or beverage formulation is a liquid, solid, semi-solid, or an emulsion.

In certain example embodiments, the dietary supplement, feed, or foodstuff or beverage formulation is adapted for daily, every other day, weekly, or monthly administration.

In certain example embodiments, the subject in need thereof is a mammal, optionally a human.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1—Shows the experimental design for Example 1. Mice were randomly assigned to their treatment group based on genotype over the 1.5 year study. Following the study baseline measures during the pre-week period, the standard chow diet was changed to study diet and continued for up to 14 weeks or until euthanasia.

FIG. 2A-2F—Shows the overall CLA study effects on Week 12 weight, fat, lean mass, temperature, and food intake. Pst-hoc analysis findings for (FIG. 2A-2B) bodyweight, (FIG. 2C) fat, (FIG. 2D) lean mass, (FIG. 2E) temperature, and (FIG. 2F) food intake, from Week 12 data points for the 12-week study. All data are presented as mean+/−the standard error of the mean. N=8 WT control, 8 WT CLA, 8 PWS control, 8 PWS CLA, 7 PWS-KO control, 8 PWS-KO CLA. ***p<0.001, **p<0.01, *p<0.05. Individual statistics for each measure are provided in Example 1. WT=(Snord116^{m+/p+}), PWS=(Snord116^{m+/p-}), PWS-KO−(Snord116^{m-/p-}).

FIG. 3A-3F—Shows Fasting glucose and glucose tolerance findings with CLA diet. Glucose measurements were performed in the week prior to diet treatment and at the end of the study (12 weeks). Post hoc analysis findings for fasting glucose (FIG. 3A) for both study times, (FIG. 3B) glucose tolerance for each genotype pre-study, (FIG. 3C) area-under-the-curve values for each genotype, pre-study, (FIG. 3D) glucose tolerance curves for each genotype on control diet, (FIG. 3E) glucose tolerance curves for each genotype on CLA diet, and (FIG. 3F) area-under-the-curve values for each genotype and treatment group. All data are presented as mean+/−standard error of the mean. N=8 WT control, 8 WT CLA, 8 PWS control, 7 PWS CLA, 7 PWS-KO control, 8 PWS-KO CLA. Letters indicate significant differences within a single figure for effects of genotype. For effect of treatment, * indicates an effect of treatment at the p<0.05 level, ** indicates an effect of treatment at the p<0.01 levels, and NS indicates the differences are not significant. Individual statistics for each measure are provided in Example 1. WT=(Snord116m+/p+), PWS=(Snord116m+/p−). PWS-KO=(Snord116m−/p−).

FIG. 4A-4B—Shows metabolic rate in control and CLA-treated animals. TSE system measurements were per-formed at the end of the 12-week study. Effect tests of genotype, treatment, and genotype×treatment were all non-significant. (FIG. 4A) Respiratory Exchange Ratio (VCO₂/VO₂) and (FIG. 4B) Energy Expenditure (KJ/kg/FFM/h). Due to a broken/malfunctioning part that occurred during the study, only the following animal numbers from the entire study were tested in the TSE system: N=7 WT control, N=5 WT CLA, N=6 PWS control, N=6 PWS CLA, N=4 PWS-KO control, N=4 PWS-KO CLA. Individual statistics for each measure are provided in Example 1. WT=(Snord116^{m+/p+}), PWS=(Snord116^{m+/p-}), PWS-KO−(Snord116^{m-/p-}).

FIG. 5A-5D—Shows activity measures in control and CLA-treated mice. (FIG. 5A) Mice were placed in cages containing running wheels for 72 h, with the first 24 h of running discounted for acclimation to the new cage. Wheel running activity was measured in both the pre- and post-study time periods. Twenty-four-hour measurements were collected. N=8 WT control, N=8 WT CLA, N=7 PWS control, N=6 PWS CLA, N=8 PWS-KO control, N=7 PWS-KO CLA. (FIG. 5B) Home-cage activity in the X—Y—Z axes was collected via beam breaks during the calorimetry measurement in the post-study period. The software converts these readings to meters per hour. N=7 WT control, N=5 WT CLA, N=6 PWS control, N=6 PWS CLA, N=4 PWS-KO control, N=4 PWS-KO CLA. (FIG. 5C) Following three days of rotarod acclimation, fourth-day rotarod tests were conducted in the post-study period. The average of four tests for each animal is shown. N=8 WT control, N=8 WT CLA, N=7 PWS control, N=6 PWS CLA, N=8 PWS-KO control, N=7 PWS-KO CLA. (FIG. D) Time in open arms was determined with a 5 min testing period. N=8 WT control, N=8 WT CLA, N=6 PWS control, N=6 PWS CLA, N=8 PWS-KO control, N=6 PWS-KO CLA, NS-non-significant. For effect of treatment, ** indicates an effect of treatment at the p<0.01 level. Individual statistics for each measure are provided Example 1. WT=(Snord116m+/p+). PWS=(Snord116m+/p−), PWS-KO=(Snord116m−/p−).

FIG. 6A-6C—Shows in vivo plantarflexor torque measurements. (FIG. 6A) Torque frequency, normalized to total body mass (TBM). *WT CLA>PWS, PWS CLA, PWS-KO. **PWS-KO<PWS-KO CLA. ***WT<WT CLA, PWS, PWS CLA, PWS-KO CLA. (FIG. 6B) Torque-frequency, normalized to Week 12 lean body mass (LBM) to account for differences in total body weight. *PWS>PWS CLA. **WT<WT CLA, PWS, PWS-KO CLA. (FIG. 6C) Fatigue as percent of initial contraction peak. *PWS-KO>all other groups. **PWS>WT. WT CLA, PWS CLA. ***PWS-KO CLA>WT CLA, PWS CLA. Comparisons p<0.05. N=8 WT control, N=8 WT CLA, N=8 PWS control, N=7 PWS CLA, N=8 PWS-KO control, N=7 PWS-KO CLA. WT=(Snord116m+/p+), PWS=(Snord116m+/p−), PWS-KO=(Snord116m−/p−).

FIG. 7A-7H—Shows Liver histology in control and CLA-diet-treated mice. Liver biopsies were obtained from all mice that completed the study at necropsy. N=3 samples from each genotype and treatment group were prepared for histological analysis with H&E staining. Representatives from all samples examined are shown at 10× magnification. (FIG. 7A) WT control diet, N=8; (FIG. 7B) PWS control diet, N=8; (FIG. 7C) PWS-KO control diet, N=7; (FIG. 7D) WT CLA diet, N=8; (FIG. 7E) PWS CLA diet showing liver steatosis, N=5: (FIG. 7F) PWS CLA diet showing normal liver morphology, N=3; (FIG. 7G) PWS-KO CLA diet showing liver steatosis, N=4; (FIG. 7H) PWS-KO CLA diet showing normal liver morphology, N=3. WT=(Snord116m+/p+), PWS=(Snord116m+/p−), PWS-KO=(Snord116m−/p−).

FIG. 8A-8C—RNA-seq analysis and results in CLA-treated and untreated WT and PWS animals. At the end of the study, hypothalamic RNA was isolated from PWS and WT animals from both diet groups. RNA-seq analysis was performed on N=5 (WT, control and CLA diet, PWS CLA diet) and N=4 (PWS control diet). (FIG. 8A) Differentially regulated genes (DEGs) in all possible cross-conditions. All differentially expressed genes are listed in Supplementary Data Files S1, S2 and S3 of Knott et al. 2022. Nutrients. 14:860. Significant GO terms are indicated (Tables 2-4 of Example 1 herein). (FIG. 8B) Two genes that were differentially regulated between WT and PWS mice, regardless of diet, are shown. (FIG. 8C) Two genes that were differentially regulated with diet (no genotype effect) are shown. ns=non-significant; *=p<0.05. N=9-11 samples for QPCR per analysis. Graphs show geometric mean±geometric SD, normalized to WT animals (FIG. 8B) or control diets (FIG. 8C). WT=(Snord116m+/p+), PWS=(Snord116m+/p−), PWS-KO=(Snord116m−/p−).

FIG. 9A-9C—Shows changes in bacterial diversity in mice fed control and CLA diets. DNA isolated from cecal contents was used for 16S IRNA sequencing. (FIG. 9A) Principal coordinate analysis (PCoA) displayed no clustering of the bacterial communities by treatment or genotype; however, bacterial communities of mice clustered by diet. (FIG. 9B) At the phyla level, the relative abundance of Cyanobacteria differed by diet (p=0.04). (FIG. 9C) At genus level, the relative abundance of Ruminococcus (p=0.02), Sutterela (p=0.01), and Turicibacter (p=0.007) differed by diet. The Kruskal-Wallis test was used to determine the overall effects of diet, genotype, and their interaction. WT=(Snord116m+/p+), PWS=(Snord116m+/p−), PWS-KO=(Snord116m−/p−).

FIG. 10A-10H—Shows whole study effects on weight, fat, lean mass, temperature, and food intake. Post hoc analysis findings for (FIG. 10A) body weight-effect of genotype (both treatments), (FIG. 10B) body weight-effect of treatment (all genotypes), (FIG. 10C) body fat-effect of treatment (all genotypes), (FIG. 10D) lean mass-effect of genotype (all treatments), (FIG. 10E) temperature-effect of treatment (all genotypes), (FIG. 10F) food intake-effect of genotype (both treatments). (FIG. 10G) food intake/gram body weight-effect of genotype, and (FIG. 10H) food intake/gram body weight-effect of treatment. All data are presented as mean+/−standard error of the mean. N=8 WT control, 8 WT CLA, 8 PWS control, 7 PWS CLA, 7 PWS-KO control, 8 PWS-KO CLA. Letters indicate significant differences within a single figure for effects of genotype. For effect of treatment, ***P<0.001, *P<0.05. Individual statistics for each measure are provided in Example 1. ˜˜ used to indicate y-axis that are not at 0).

FIG. 11—Shows fasting glucose with CLA diet-effect of treatment. Glucose measurements were done at the end of the study (12 weeks). Post hoc analysis findings for fasting glucose revealed high significance (***=P<0.001) for the effect of diet (genotypes groups by diet). Data are presented as mean+/−standard error of the mean. N=8 WT control, 8 WT CLA, 8 PWS control, 7 PWS CLA, 7 PWS-KO control, 8 PWS-KO CLA, grouped by diet.

FIG. 12A-12B—Shows wheel running post measures. (FIG. 12A) Post hoc analysis of genotype effects on wheel running were significant (P=0.0008) (FIG. 12B) Treatment effects were non-significant (NS). N=8 WT control, N=8 WT CLA, N=7 PWS control, N=6 PWS CLA, N=8 PWS-KO control, N=7 PWS-KO CLA. ˜˜ used to indicate y-axis that are not at 0.

FIG. 13—Shows the analysis of alpha diversity in microbiome. Genotype and treated effects, and interactions are shown for each analysis. *=P<0.05, for diet interactions. The actual P-values are shown below each figure.

FIG. 14A-14C—Shows body weight and fat reduction in CLA-treated animals. (FIG. 14A) Body weight (g) shows a significant reduction after 12 weeks CLA treatment as compared to control diets, regardless of genotype. (FIG. 14B) Body fat (g) shows a significant reduction after 12 weeks CLA treatment compared to control diets. (FIG. 14C) Lean mass (g) did not change between treatment groups.

FIG. 15A-15C—Shows improved RotaRod function with CLA treatment. (FIG. 15A) The rotarod apparatus showing three test lanes and one lane with grip tape for the initial acclimation. (FIG. 15B) C57Bl/6 background mice representing the WT, PWS, and PWS-KO genotypes. (FIG. 15C) 129v/J background mice, representing a genetically obese line of mice with a deletion of Nhlh2.

FIG. 16A-16C—Shows anxiety improvement with CLA treatment. (FIG. 16A) Elevated plus maze used in this experiment. (FIG. 16B) C57Bl/6 strain normal mice, and mice containing the PWS deletions. (FIG. 16C) 129v/J strain normal mice and mice containing the Nhlh2 deletion.

FIG. 17A-17C—Show body weight and fat reduction in CLA-treated animals. (FIG. 17A) Body weight (g) showed a significant reduction after 12 weeks CLA treatment as compared to control diets, regardless of genotype. (FIG. 17B) Body fat (g) showed a significant reduction after 12 weeks CLA treatment compared to control diets. (FIG. 17C) Lean mass (g) was significantly improved in CLA-treated mice.

FIG. 18—Shows H&E stained microscopy images demonstrating improvement of testicular histology with CLA treatment. Images are at 10× magnification. Con: control treatment; CLA: CLA treatment for normal mice (129-WT) or mice with a deletion of Nhlh2 (N2KO). Inset images show sections of the same slide/image at 20× magnification.

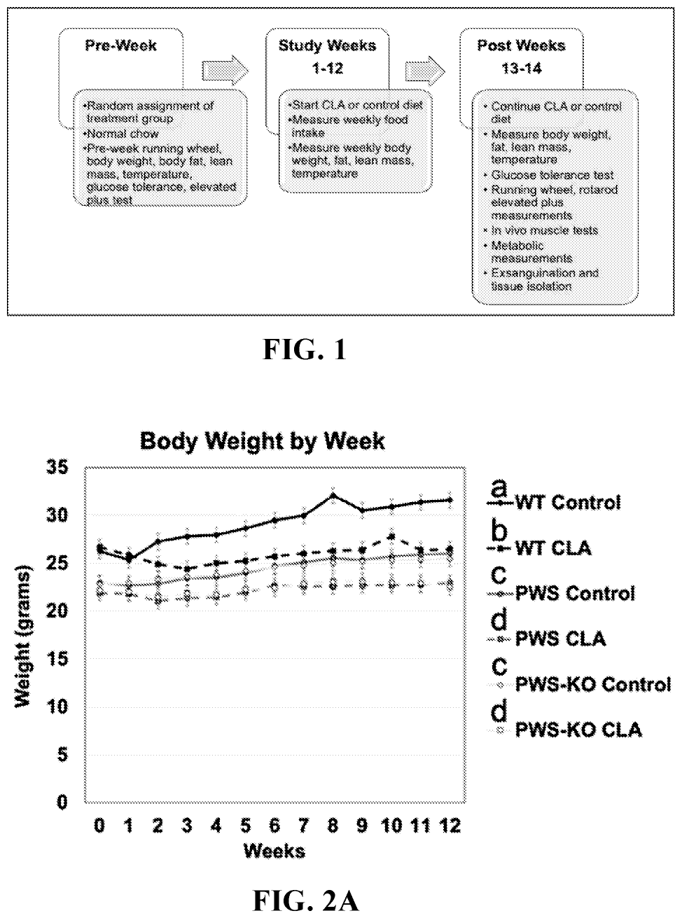

The figures herein are for illustrative purposes only and are not necessarily drawn to scale.

### DETAILED DESCRIPTION OF THE EXAMPLE EMBODIMENTS

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2^{nd} ed., J. Wiley & Sons (New York, N.Y. 1994), March, Advanced Organic Chemistry Reactions, Mechanisms and Structure 4^{th} ed., John Wiley & Sons (New York, N.Y. 1992); and Marten H. Hofker and Jan van Deursen, Transgenic Mouse Methods and Protocols, 2^{nd} edition (2011).

Definitions of common terms and techniques in chemistry and organic chemistry can be found in Smith. Organic Synthesis, published by Academic Press. 2016; Tinoco et al. Physical Chemistry, 5^{th} edition (2013) published by Pearson; Brown et al., Chemistry, The Central Science 14^{th} ed. (2017), published by Pearson, Clayden et al., Organic Chemistry, 2^{nd} ed. 2012, published by Oxford University Press; Carey and Sunberg, Advanced Organic Chemistry, Part A: Structure and Mechanisms, 5^{th} ed. 2008, published by Springer; Carey and Sunberg, Advanced Organic Chemistry, Part B: Reactions and Synthesis, 5^{th} ed. 2010, published by Springer, and Vollhardt and Schore, Organic Chemistry, Structure and Function; 8^{th} ed. (2018) published by W. H. Freeman.

Definitions of common terms, analysis, and techniques in genetics can be found in e.g., Hartl and Clark. Principles of Population Genetics. 4^{th} Ed. 2006, published by Oxford University Press. Published by Booker. Genetics: Analysis and Principles, 7^{th} Ed. 2021, published by McGraw Hill; Isik et la., Genetic Data Analysis for Plant and Animal Breeding. First ed. 2017. published by Springer International Publishing AG; Green, E. L. Genetics and Probability in Animal Breeding Experiments. 2014, published by Palgrave; Bourdon, R. M. Understanding Animal Breeding. 2000 2^{nd} Ed. published by Prentice Hall; Pal and Chakravarty. Genetics and Breeding for Disease Resistance of Livestock. First Ed. 2019, published by Academic Press; Fasso, D. Classification of Genetic Variance in Animals. First Ed. 2015, published by Callisto Reference; Megahed, M. Handbook of Animal Breeding and Genetics, 2013, published by Omniscriptum Gmbh & Co. Kg., LAP Lambert Academic Publishing; Reece. Analysis of Genes and Genomes. 2004, published by John Wiley & Sons. Inc; Deonier et al., Computational Genome Analysis. 5^{th} Ed. 2005, published by Springer-Verlag, New York; Meneely, P. Genetic Analysis: Genes, Genomes, and Networks in Eukaryotes. 3^{rd} Ed. 2020, published by Oxford University Press.

As used herein, the singular forms “a”, “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g. a given confidence interval (e.g. 90%, 95%, or more confidence interval from the mean), such as variations of +/−10% or less, +/−5% or less, +/−1% or less, and +/−0.1% or less of and from the specified value, insofar such variations are appropriate to perform in the disclosed invention. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

As used herein, a “biological sample” refers to a sample obtained from, made by, secreted by, excreted by, or otherwise containing part of or from a biologic entity. A biologic sample can contain whole cells and/or live cells and/or cell debris, and/or cell products, and/or virus particles. The biological sample can contain (or be derived from) a “bodily fluid”. The biological sample can be obtained from an environment (e.g., water source, soil, air, and the like). Such samples are also referred to herein as environmental samples. As used herein “bodily fluid” refers to any non-solid excretion, secretion, or other fluid present in an organism and includes, without limitation unless otherwise specified or is apparent from the description herein, amniotic fluid, aqueous humor, vitreous humor, bile, blood or component thereof (e.g. plasma, serum, etc.), breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from an organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

As used herein, “administering” refers to any suitable administration for the agent(s) being delivered and/or subject receiving said agent(s) and can be oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example, a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration routes can be, for instance, auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratym panic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated, subject being treated, and/or agent(s) being administered.

As used herein, “agent” refers to any substance, compound, molecule, and the like, which can be administered to a subject on a subject to which it is administered to. An agent can be inert. An agent can be an active agent. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

As used herein, “differentially expressed,” refers to the differential production of RNA, including but not limited to mRNA, tRNA, miRNA, siRNA, snRNA, and piRNA transcribed from a gene or regulatory region of a genome or the protein product encoded by a gene as compared to the level of production of RNA or protein by the same gene or regulator region in a normal or a control cell. In another context, “differentially expressed,” also refers to nucleotide sequences or proteins in a cell or tissue which have different temporal and/or spatial expression profiles as compared to a normal or control cell.

As used herein, the terms “disease” or “disorder” are used interchangeably throughout this specification, and refer to any alternation in state of the body or of some of the organs, interrupting or disturbing the performance of the functions and/or causing symptoms such as discomfort, dysfunction, distress, or even death to the person afflicted or those in contact with a person. A disease or disorder can also be related to a distemper, ailing, ailment, malady, disorder, sickness, illness, complaint, indisposition, or affliction.

As used herein, “dose,” “unit dose,” or “dosage” can refer to physically discrete units suitable for use in a subject, each unit containing a predetermined quantity of the CLA or formulation thereof calculated to produce the desired response or responses or deliver the desired amount of the CLA in association with its administration.

As used herein, “expression” refers to the process by which polynucleotides are transcribed into RNA transcripts. In the context of mRNA and other translated RNA species, “expression” also refers to the process or processes by which the transcribed RNA is subsequently translated into peptides, polypeptides, or proteins. In some instances, “expression” can also be a reflection of the stability of a given RNA. For example, when one measures RNA, depending on the method of detection and/or quantification of the RNA as well as other techniques used in conjunction with RNA detection and/or quantification, it can be that increased/decreased RNA transcript levels are the result of increased/decreased transcription and/or increased/decreased stability and/or degradation of the RNA transcript. One of ordinary skill in the art will appreciate these techniques and the relation “expression” in these various contexts to the underlying biological mechanisms.

As used herein, “gene” refers to a hereditary unit corresponding to a sequence of DNA that occupies a specific location on a chromosome and that contains the genetic instruction for a characteristic(s) or trait(s) in an organism. The term gene can refer to translated and/or untranslated regions of a genome. “Gene” can refer to the specific sequence of DNA that is transcribed into an RNA transcript that can be translated into a polypeptide or be a catalytic RNA molecule, including but not limited to, tRNA, siRNA, piRNA, miRNA, long-non-coding RNA and shRNA.

As used herein “increased expression” or “overexpression” are both used to refer to an increased expression of a gene, such as a gene relating to an antigen processing and/or presentation pathway, or gene product thereof in a sample as compared to the expression of said gene or gene product in a suitable control. The term “increased expression” preferably refers to 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 610%, 620%, 630%, 640%, 650%, 660%, 670%, 680%, 690%, 700%, 710%, 720%, 730%, 740%, 750%, 760%, 770%, 780%, 790%, 800%, 810%, 820%, 830%, 840%, 850%, 860%, 870%, 880%, 890%, 900%, 910%, 920%, 930%, 940%, 950%, 960%, 970%, 980%, 990%, 1000%, 1010%, 1020%, 1030%, 1040%, 1050%, 1060%, 1070%, 1080%, 1090%, 1100%, 1110%, 1120%, 1130%, 1140%, 1150%, 1160%, 1170%, 1180%, 1190%, 1200%, 1210%, 1220%, 1230%, 1240%, 1250%, 1260%, 1270%, 1280%, 1290%, 1300%, 1310%, 1320%, 1330%, 1340%, 1350%, 1360%, 1370%, 1380%, 1390%, 1400%, 1410%, 1420%, 1430%, 1440%, 1450%, 1460%, 1470%, 1480%, 1490%, or/to 1500% or more increased expression relative to a suitable control.

As used herein “reduced expression”, “decreased expression”, or “underexpression” are used interchangeably herein and refer to a reduced or decreased expression of a gene, such as a gene relating to an antigen processing pathway, or a gene product thereof in sample as compared to the expression of said gene or gene product in a suitable control. As used herein throughout this specification, “suitable control” is a control that will be instantly appreciated by one of ordinary skill in the art as one that is included such that it can be determined if the variable being evaluated an effect, such as a desired effect or hypothesized effect. One of ordinary skill in the art will also instantly appreciate based on inter alia, the context, the variable(s), the desired or hypothesized effect, what is a suitable or an appropriate control needed. In one embodiment, said control is a sample from a healthy individual or otherwise normal individual. By way of a non-limiting example, if said sample is a sample of a lung tumor and contains lung tissue, said control is lung tissue of a healthy individual. The term “reduced expression” and/or the like refers to at least a 25% reduction, e.g., at least a 30%, 40%, 50%, 60%, 70%, 75%, 80%, 85%, 90%, 95%, 98% or 99% reduction, relative to such control or as otherwise described in this specification.

As used interchangeably herein, the terms “sufficient” and “effective,” can refer to an amount (e.g. mass, volume, dosage, concentration, and/or time period) needed to achieve one or more desired result(s). For example, a therapeutically effective amount refers to an amount needed to achieve one or more therapeutic effects. Exemplary therapeutic effects ascribed to an effective amount are discussed in greater detail elsewhere herein.

As used herein, “modulate” broadly denotes a qualitative and/or quantitative alteration, change or variation in that which is being modulated. Where modulation can be assessed quantitatively—for example, where modulation comprises or consists of a change in a quantifiable variable such as a quantifiable property of a cell or where a quantifiable variable provides a suitable surrogate for the modulation—modulation specifically encompasses both increase (e.g., activation) or decrease (e.g., inhibition) in the measured variable. The term encompasses any extent of such modulation, e.g., any extent of such increase or decrease, and may more particularly refer to statistically significant increase or decrease in the measured variable. By means of example, in aspects modulation may encompass an increase in the value of the measured variable by about 10 to 500 percent or more. In aspects, modulation can encompass an increase in the value of at least 10%, 20%, 30%, 40%, 50%, 75%, 100%, 150%, 200%, 250%, 300%, 400% to 500% or more, compared to a reference situation or suitable control without said modulation. In aspects, modulation may encompass a decrease or reduction in the value of the measured variable by about 5 to about 100%. In some aspects, the decrease can be about 5%, 10%, 20%, 30%, 40%, 50%, 60%, 70%, 80%, 90%, 95%, 96%, 97%, 98%, 99% to about 100%, compared to a reference situation or suitable control without said modulation. In some embodiments, modulation can be stimulated by an exogenous actor, such as a drug, diet, abiotic environmental factor.

As used herein, “negative control” refers to a “control” that is designed to produce no effect or result, provided that all reagents are functioning properly and that the experiment is properly conducted. Other terms that are interchangeable with “negative control” include “sham,” “placebo,” and “mock.”

As used herein, “positive control” refers to a “control” that is designed to produce the desired result, provided that all reagents are functioning properly and that the experiment is properly conducted.

As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo.

As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient.

As used herein, “tangible medium of expression” refers to a medium that is physically tangible or accessible and is not a mere abstract thought or an unrecorded spoken word. “Tangible medium of expression” includes, but is not limited to, words on a cellulosic or plastic material, or data stored in a suitable computer readable memory form. The data can be stored on a unit device, such as a flash memory or CD-ROM or on a server that can be accessed by a user via, e.g., a web interface.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as a Snord116 deficiency disease or a Nhlh2 deficiency associated hypogonadism or symptom thereof. The effect can be therapeutic in terms of a partial or complete cure of a disease, condition, symptom or adverse effect attributed to the disease, disorder, or condition. The term “treatment” as used herein covers any treatment of a Snord116 deficiency disease or a Nhlh2 deficiency associated hypogonadism or symptom thereof, in a subject, particularly a human and can include any one or more of the following: (a) preventing the disease or a symptoms from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, such as treating the pain of a subject by administration of an analgesic agent even though such agent does not treat the cause of the pain.

As used herein, “nutraceutical” refers to a dietary supplement that are composed of extracts, concentrates or combinations of vitamins, minerals, botanicals, herbs, or dietary substances “for use by man to supplement the diet by increasing the total dietary intake”.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Prader-Willi Syndrome (PWS) is a genetic condition that occurs in up to 1 in 10,000 live births. Individuals with PWS show initial developmental delay, significant hypotonia/muscle weakness, and typically demonstrate some level of intellectual disability, and obesity later in childhood or adolescence. The most common cause of the syndrome is a de novo deletion of the paternal 15q chromosome, as the maternal allele is imprinted and not expressed. Uniparental (maternal) disomy and an imprinting locus mutation can also be causative. A minimal deletion of chromosome 15q that causes PWS includes just two expressed regions; the SNORD116 cluster (a group of 28 or more small nucleolar RNAs “snoRNAs”), and the IPW gene which also encodes a non-coding RNA of little known function. SNORD116 regulates expression of Nhlh2. More specifically, in normal individuals, SNORD116 stimulates Nhlh2 expression. In PWS, Nhlh2 expression is decreased, which is consistent with SNORD116 deletion and subsequent reduction in SNORD116 stimulation of Nhlh2 expression. For PWS patients, there is, as yet, no cure, and few treatment options. As such, there at least a need for improved treatments of PWS and/or symptoms thereof.

With that said, embodiments disclosed herein can provide methods for treating a Snord116 deficiency, disease or a symptom thereof (e.g., PWS or symptom thereof), hypogonadism associated with an Nhlh2 deficiency or disease thereof, or both in a subject that include administering an effective amount of CLA or a formulation thereof to the subject. Also described in several example embodiments herein are formulations, such as dietary supplements, feed, or food stuffs that comprise an effective amount of CLA or formulation thereof. Such dietary supplements, feed, and/or foodstuff or beverage can be administered to a subject in need thereof for treating a Snord116 deficiency, disease or a symptom thereof (e.g., PWS or symptom thereof), hypogonadism associated with an Nhlh2 deficiency or disease thereof, or both in a subject to which they are administered. Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### CLA Formulations

Described herein are formulations, such as dietary supplements, feed formulations, foodstuffs and beverages, and/or the like, the contain an amount of CLA or formulations such that, when administered to a subject in need thereof, the total amount of CLA administered in one or more doses of the formulation is an effective amount of CLA to treat a Snord116 deficiency disease or a symptom thereof (e.g., PWS) and/or a Nhlh2 deficiency associated hypogonadism or symptom thereof. As used herein “Snord116 deficiency disease” refers to any disease or condition that is caused at least in part or in whole by a mutation, deletion, insertion, or any combination thereof of one or both copies of the Snord116 gene or locus such that the expression or function of the Snord116 gene or gene product from the one or both copies of the Snord116 gene or locus is decreased or eliminated thereby causing the disease, condition, or symptom(s) thereof in a subject. As used herein “Nhlh2 deficiency associated hypogonadism” refers to hypogonadism that is caused at least in part or in whole by a mutation, deletion, insertion, or any combination thereof of one or both copies of the Nhlh2 gene or locus such that the expression or function of the Nhlh2 gene or gene product from the one or both copies of the Nhlh2 gene or locus is decreased or eliminated thereby causing the hypogonadism and/or symptom(s) thereof in a subject.

Described in certain example embodiments herein are dietary supplements, feeds, or foodstuff or beverage formulations effective for treating a Snord116 deficiency disease or a symptom thereof in a subject in need thereof, the dietary supplement comprising an amount of conjugated linoleic acid (CLA) or a formulation thereof such that the dietary supplement delivers an effective amount of the CLA to the subject in need thereof in one or more doses, optionally 1-3 doses.

Described in certain example embodiments herein are dietary supplements, feeds, or foodstuff or beverage formulations hypogonadism in a subject having a Nhlh2 deficiency, disease, or symptom thereof in a subject in need thereof, the dietary supplement comprising an amount of conjugated linoleic acid (CLA) or a formulation thereof to the subject in need thereof such that the dietary supplement delivers an effective amount of the CLA to the subject in need thereof in one or more doses, optionally 1-3 doses.

As used herein, “dietary supplement” is a composition or formulation that is in addition to the basic nutrient requirements of a human subject, that is administered in addition to the diet of the subject. Dietary supplements can be formulated such that no dilution, mixing, or other preparation is required before consumption. Dietary supplements can be formulated such that they are intended to be mixed or diluted into a foodstuff or beverage to be consumed as part of the regular diet of the subject.

As used herein, “food stuff” refers to a non-beverage food product intended to be consumed by a human subject. As used herein, “beverage” refers to a liquid composition intended to be drunk by a human subject. As used herein, the term beverage includes water, juices, milk, sodas, sports drinks, and/or the like.

In certain example embodiments, the Snord116 deficiency disease is Prader-Willi Syndrome (PWS).

In certain example embodiments, the effective amount of CLA is at least 1,000 mg per day, optionally about 3,000 to about 5,000 mg per day. In certain example embodiments, the effective amount of CLA is about 1000 mg, 1025 mg, 1050 mg, 1075 mg, 1100 mg, 1125 mg, 1150 mg, 1175 mg, 1200 mg, 1225 mg, 1250 mg, 1275 mg, 1300 mg, 1325 mg, 1350 mg, 1375 mg, 1400 mg, 1425 mg, 1450 mg, 1475 mg, 1500 mg, 1525 mg, 1550 mg, 1575 mg, 1600 mg, 1625 mg, 1650 mg, 1675 mg, 1700 mg, 1725 mg, 1750 mg, 1775 mg, 1800 mg, 1825 mg, 1850 mg, 1875 mg, 1900 mg, 1925 mg, 1950 mg, 1975 mg, 2000 mg, 2025 mg, 2050 mg, 2075 mg, 2100 mg, 2125 mg, 2150 mg, 2175 mg, 2200 mg, 2225 mg, 2250 mg, 2275 mg, 2300 mg, 2325 mg, 2350 mg, 2375 mg, 2400 mg, 2425 mg, 2450 mg, 2475 mg, 2500 mg, 2525 mg, 2550 mg, 2575 mg, 2600 mg, 2625 mg, 2650 mg, 2675 mg, 2700 mg, 2725 mg, 2750 mg, 2775 mg, 2800 mg, 2825 mg, 2850 mg, 2875 mg, 2900 mg, 2925 mg, 2950 mg, 2975 mg, 3000 mg, 3025 mg, 3050 mg, 3075 mg, 3100 mg, 3125 mg, 3150 mg, 3175 mg, 3200 mg, 3225 mg, 3250 mg, 3275 mg, 3300 mg, 3325 mg, 3350 mg, 3375 mg, 3400 mg, 3425 mg, 3450 mg, 3475 mg, 3500 mg, 3525 mg, 3550 mg, 3575 mg, 3600 mg, 3625 mg, 3650 mg, 3675 mg, 3700 mg, 3725 mg, 3750 mg, 3775 mg, 3800 mg, 3825 mg, 3850 mg, 3875 mg, 3900 mg, 3925 mg, 3950 mg, 3975 mg, 4000 mg, 4025 mg, 4050 mg, 4075 mg, 4100 mg, 4125 mg, 4150 mg, 4175 mg, 4200 mg, 4225 mg, 4250 mg, 4275 mg, 4300 mg, 4325 mg, 4350 mg, 4375 mg, 4400 mg, 4425 mg, 4450 mg, 4475 mg, 4500 mg, 4525 mg, 4550 mg, 4575 mg, 4600 mg, 4625 mg, 4650 mg, 4675 mg, 4700 mg, 4725 mg, 4750 mg, 4775 mg, 4800 mg, 4825 mg, 4850 mg, 4875 mg, 4900 mg, 4925 mg, 4950 mg, 4975 mg, 5000 mg, 5025 mg, 5050 mg, 5075 mg, 5100 mg, 5125 mg, 5150 mg, 5175 mg, 5200 mg, 5225 mg, 5250 mg, 5275 mg, 5300 mg, 5325 mg, 5350 mg, 5375 mg, 5400 mg, 5425 mg, 5450 mg, 5475 mg, 5500 mg, 5525 mg, 5550 mg, 5575 mg, 5600 mg, 5625 mg, 5650 mg, 5675 mg, 5700 mg, 5725 mg, 5750 mg, 5775 mg, 5800 mg, 5825 mg, 5850 mg, 5875 mg, 5900 mg, 5925 mg, 5950 mg, 5975 mg, 6000 mg, 6025 mg, 6050 mg, 6075 mg, 6100 mg, 6125 mg, 6150 mg, 6175 mg, 6200 mg, 6225 mg, 6250 mg, 6275 mg, 6300 mg, 6325 mg, 6350 mg, 6375 mg, 6400 mg, 6425 mg, 6450 mg, 6475 mg, 6500 mg, 6525 mg, 6550 mg, 6575 mg, 6600 mg, 6625 mg, 6650 mg, 6675 mg, 6700 mg, 6725 mg, 6750 mg, 6775 mg, 6800 mg, 6825 mg, 6850 mg, 6875 mg, 6900 mg, 6925 mg, 6950 mg, 6975 mg, 7000 mg, 7025 mg, 7050 mg, 7075 mg, 7100 mg, 7125 mg, 7150 mg, 7175 mg, 7200 mg, 7225 mg, 7250 mg, 7275 mg, 7300 mg, 7325 mg, 7350 mg, 7375 mg, 7400 mg, 7425 mg, 7450 mg, 7475 mg, 7500 mg, 7525 mg, 7550 mg, 7575 mg, 7600 mg, 7625 mg, 7650 mg, 7675 mg, 7700 mg, 7725 mg, 7750 mg, 7775 mg, 7800 mg, 7825 mg, 7850 mg, 7875 mg, 7900 mg, 7925 mg, 7950 mg, 7975 mg, 8000 mg, 8025 mg, 8050 mg, 8075 mg, 8100 mg, 8125 mg, 8150 mg, 8175 mg, 8200 mg, 8225 mg, 8250 mg, 8275 mg, 8300 mg, 8325 mg, 8350 mg, 8375 mg, 8400 mg, 8425 mg, 8450 mg, 8475 mg, 8500 mg, 8525 mg, 8550 mg, 8575 mg, 8600 mg, 8625 mg, 8650 mg, 8675 mg, 8700 mg, 8725 mg, 8750 mg, 8775 mg, 8800 mg, 8825 mg, 8850 mg. 8875 mg, 8900 mg, 8925 mg, 8950 mg, 8975 mg, 9000 mg, 9025 mg, 9050 mg, 9075 mg, 9100 mg, 9125 mg, 9150 mg, 9175 mg, 9200 mg, 9225 mg, 9250 mg, 9275 mg, 9300 mg, 9325 mg, 9350 mg, 9375 mg, 9400 mg, 9425 mg, 9450 mg, 9475 mg, 9500 mg, 9525 mg, 9550 mg, 9575 mg, 9600 mg, 9625 mg, 9650 mg, 9675 mg, 9700 mg, 9725 mg, 9750 mg, 9775 mg, 9800 mg, 9825 mg, 9850 mg, 9875 mg, 9900 mg, 9925 mg, 9950 mg, 9975 mg, to/or 10000 mg. In certain example embodiments, the effective amount of CLA is about 3000 mg, 3025 mg, 3050 mg, 3075 mg, 3100 mg, 3125 mg, 3150 mg, 3175 mg, 3200 mg, 3225 mg, 3250 mg, 3275 mg, 3300 mg, 3325 mg, 3350 mg, 3375 mg, 3400 mg, 3425 mg, 3450 mg, 3475 mg, 3500 mg, 3525 mg, 3550 mg, 3575 mg, 3600 mg, 3625 mg, 3650 mg, 3675 mg, 3700 mg, 3725 mg, 3750 mg, 3775 mg, 3800 mg, 3825 mg, 3850 mg, 3875 mg, 3900 mg, 3925 mg, 3950 mg, 3975 mg, 4000 mg, 4025 mg, 4050 mg, 4075 mg, 4100 mg, 4125 mg, 4150 mg, 4175 mg, 4200 mg, 4225 mg, 4250 mg, 4275 mg, 4300 mg, 4325 mg, 4350 mg, 4375 mg, 4400 mg, 4425 mg, 4450 mg, 4475 mg, 4500 mg, 4525 mg, 4550 mg, 4575 mg, 4600 mg, 4625 mg, 4650 mg, 4675 mg, 4700 mg, 4725 mg, 4750 mg, 4775 mg, 4800 mg, 4825 mg, 4850 mg, 4875 mg, 4900 mg, 4925 mg, 4950 mg, 4975 mg, to/or about 5000 mg. In certain example embodiments, the effective amount of CLA is about 3000 mg.

In certain example embodiments, the effective amount of the CLA is about 3-10 g/kg body weight per day, optionally about 5 g/kg body weight per day. In certain example embodiments, the effective amount of the CLA is about 3 g/kg body weight, 3.1 g/kg body weight, 3.2 g/kg body weight, 3.3 g/kg body weight, 3.4 g/kg body weight, 3.5 g/kg body weight, 3.6 g/kg body weight, 3.7 g/kg body weight, 3.8 g/kg body weight, 3.9 g/kg body weight, 4 g/kg body weight, 4.1 g/kg body weight, 4.2 g/kg body weight, 4.3 g/kg body weight, 4.4 g/kg body weight, 4.5 g/kg body weight, 4.6 g/kg body weight, 4.7 g/kg body weight, 4.8 g/kg body weight, 4.9 g/kg body weight, 5 g/kg body weight, 5.1 g/kg body weight, 5.2 g/kg body weight, 5.3 g/kg body weight, 5.4 g/kg body weight, 5.5 g/kg body weight, 5.6 g/kg body weight, 5.7 g/kg body weight, 5.8 g/kg body weight, 5.9 g/kg body weight, 6 g/kg body weight, 6.1 g/kg body weight, 6.2 g/kg body weight, 6.3 g/kg body weight, 6.4 g/kg body weight, 6.5 g/kg body weight, 6.6 g/kg body weight, 6.7 g/kg body weight, 6.8 g/kg body weight, 6.9 g/kg body weight, 7 g/kg body weight, 7.1 g/kg body weight, 7.2 g/kg body weight, 7.3 g/kg body weight, 7.4 g/kg body weight, 7.5 g/kg body weight, 7.6 g/kg body weight, 7.7 g/kg body weight, 7.8 g/kg body weight, 7.9 g/kg body weight, 8 g/kg body weight, 8.1 g/kg body weight, 8.2 g/kg body weight, 8.3 g/kg body weight, 8.4 g/kg body weight, 8.5 g/kg body weight, 8.6 g/kg body weight, 8.7 g/kg body weight, 8.8 g/kg body weight, 8.9 g/kg body weight, 9 g/kg body weight, 9.1 g/kg body weight, 9.2 g/kg body weight, 9.3 g/kg body weight, 9.4 g/kg body weight, 9.5 g/kg body weight, 9.6 g/kg body weight, 9.7 g/kg body weight, 9.8 g/kg body weight, 9.9 g/kg body weight, to/or 10 g/kg body weight. In certain example embodiments, the effective amount of the CLA is about 4.5 g/kg body weight, 4.6 g/kg body weight, 4.7 g/kg body weight, 4.8 g/kg body weight, 4.9 g/kg body weight, 5 g/kg body weight, 5.1 g/kg body weight, 5.2 g/kg body weight, 5.3 g/kg body weight, 5.4 g/kg body weight, to/or about 5.5 g/kg body weight. In certain example embodiments, the effective amount of the CLA is about 5.0 g/kg body weight, 5.1 g/kg body weight, to/or 5.2 g/kg body weight.

In certain example embodiments, the CLA or formulation thereof contains or is composed entirely of a mixture of CLA isoforms, optionally a 50:50 mixture of trans-10, cis-12 and trans9, cis-11 CLA isomers. In certain example embodiments, the CLA or formulation thereof contains 80-100% of a mixture of CLA isoforms. In some embodiments, the mixture of CLA isoforms is a 50:50 mixture of trans-10, cis-12 and trans9, cis-11 CLA isomers. In some embodiments, the CLA or formulation thereof contains about 80%, 81%, 82%, 83%, 84%, 85%, 86%, 87%, 88%, 89%, 90%, 91%, 92%, 93%, 94%, 95%, 96%, 97%, 98%, 99%, to/or 100%, of a mixture of CLA isoforms. In some embodiments, the mixture of CLA isoforms is a 50:50 mixture of trans-10, cis-12 and trans9, cis-11 CLA isomers. In some embodiments, the CLA or formulation thereof is Tonalin™ CLA.

In certain example embodiments, the effective amount of CLA decreases body weight, decreases fat mass, increases lean body mass, improves hypogonadism or a symptom thereof, improves testicular morphology, modulates the α-diversity of the gut microbiome, modulates hypothalamic gene expression of one or more genes, decreases anxiety, increase sperm maturation, increase spermatogenesis, increase sperm differentiation, decrease testicular degeneration or any combination thereof in the subject in need thereof.

In some embodiments, the effective amount of CLA decreases body weight by about 0.1% to about 30% or more. In some embodiments, the effective amount of CLA decreases body weight by about 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 5.1%, 5.2%, 5.3%, 5.4%, 5.5%, 5.6%, 5.7%, 5.8%, 5.9%, 6%, 6.1%, 6.2%, 6.3%, 6.4%, 6.5%, 6.6%, 6.7%, 6.8%, 6.9%, 7%, 7.1%, 7.2%, 7.3%, 7.4%, 7.5%, 7.6%, 7.7%, 7.8%, 7.9%, 8%, 8.1%, 8.2%, 8.3%, 8.4%, 8.5%, 8.6%, 8.7%, 8.8%, 8.9%, 9%, 9.1%, 9.2%, 9.3%, 9.4%, 9.5%, 9.6%, 9.7%, 9.8%, 9.9%, 10%, 10.1%, 10.2%, 10.3%, 10.4%, 10.5%, 10.6%, 10.7%, 10.8%, 10.9%, 11%, 11.1%, 11.2%, 11.3%, 11.4%, 11.5%, 11.6%, 11.7%, 11.8%, 11.9%, 12%, 12.1%, 12.2%, 12.3%, 12.4%, 12.5%, 12.6%, 12.7%, 12.8%, 12.9%, 13%, 13.1%, 13.2%, 13.3%, 13.4%, 13.5%, 13.6%, 13.7%, 13.8%, 13.9%, 14%, 14.1%, 14.2%, 14.3%, 14.4%, 14.5%, 14.6%, 14.7%, 14.8%, 14.9%, 15%, 15.1%, 15.2%, 15.3%, 15.4%, 15.5%, 15.6%, 15.7%, 15.8%, 15.9%, 16%, 16.1%, 16.2%, 16.3%, 16.4%, 16.5%, 16.6%, 16.7%, 16.8%, 16.9%, 17%, 17.1%, 17.2%, 17.3%, 17.4%, 17.5%, 17.6%, 17.7%, 17.8%, 17.9%, 18%, 18.1%, 18.2%, 18.3%, 18.4%, 18.5%, 18.6%, 18.7%, 18.8%, 18.9%, 19%, 19.1%, 19.2%, 19.3%, 19.4%, 19.5%, 19.6%, 19.7%, 19.8%, 19.9%, 20%, 20.1%, 20.2%, 20.3%, 20.4%, 20.5%, 20.6%, 20.7%, 20.8%, 20.9%, 21%, 21.1%, 21.2%, 21.3%, 21.4%, 21.5%, 21.6%, 21.7%, 21.8%, 21.9%, 22%, 22.1%, 22.2%, 22.3%, 22.4%, 22.5%, 22.6%, 22.7%, 22.8%, 22.9%, 23%, 23.1%, 23.2%, 23.3%, 23.4%, 23.5%, 23.6%, 23.7%, 23.8%, 23.9%, 24%, 24.1%, 24.2%, 24.3%, 24.4%, 24.5%, 24.6%, 24.7%, 24.8%, 24.9%, 25%, 25.1%, 25.2%, 25.3%, 25.4%, 25.5%, 25.6%, 25.7%, 25.8%, 25.9%, 26%, 26.1%, 26.2%, 26.3%, 26.4%, 26.5%, 26.6%, 26.7%, 26.8%, 26.9%, 27%, 27.1%, 27.2%, 27.3%, 27.4%, 27.5%, 27.6%, 27.7%, 27.8%, 27.9%, 28%, 28.1%, 28.2%, 28.3%, 28.4%, 28.5%, 28.6%, 28.7%, 28.8%, 28.9%, 29%, 29.1%, 29.2%, 29.3%, 29.4%, 29.5%, 29.6%, 29.7%, 29.8%, 29.9%, to/or 30.0% or more.

In some embodiments, the effective amount of CLA decreases fat mass by about 0.1% to about 30% or more. In some embodiments, the effective amount of CLA decreases fat mass by about 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 5.1%, 5.2%, 5.3%, 5.4%, 5.5%, 5.6%, 5.7%, 5.8%, 5.9%, 6%, 6.1%, 6.2%, 6.3%, 6.4%, 6.5%, 6.6%, 6.7%, 6.8%, 6.9%, 7%, 7.1%, 7.2%, 7.3%, 7.4%, 7.5%, 7.6%, 7.7%, 7.8%, 7.9%, 8%, 8.1%, 8.2%, 8.3%, 8.4%, 8.5%, 8.6%, 8.7%, 8.8%, 8.9%, 9%, 9.1%, 9.2%, 9.3%, 9.4%, 9.5%, 9.6%, 9.7%, 9.8%, 9.9%, 10%, 10.1%, 10.2%, 10.3%, 10.4%, 10.5%, 10.6%, 10.7%, 10.8%, 10.9%, 11%, 11.1%, 11.2%, 11.3%, 11.4%, 11.5%, 11.6%, 11.7%, 11.8%, 11.9%, 12%, 12.1%, 12.2%, 12.3%, 12.4%, 12.5%, 12.6%, 12.7%, 12.8%, 12.9%, 13%, 13.1%, 13.2%, 13.3%, 13.4%, 13.5%, 13.6%, 13.7%, 13.8%, 13.9%, 14%, 14.1%, 14.2%, 14.3%, 14.4%, 14.5%, 14.6%, 14.7%, 14.8%, 14.9%, 15%, 15.1%, 15.2%, 15.3%, 15.4%, 15.5%, 15.6%, 15.7%, 15.8%, 15.9%, 16%, 16.1%, 16.2%, 16.3%, 16.4%, 16.5%, 16.6%, 16.7%, 16.8%, 16.9%, 17%, 17.1%, 17.2%, 17.3%, 17.4%, 17.5%, 17.6%, 17.7%, 17.8%, 17.9%, 18%, 18.1%, 18.2%, 18.3%, 18.4%, 18.5%, 18.6%, 18.7%, 18.8%, 18.9%, 19%, 19.1%, 19.2%, 19.3%, 19.4%, 19.5%, 19.6%, 19.7%, 19.8%, 19.9%, 20%, 20.1%, 20.2%, 20.3%, 20.4%, 20.5%, 20.6%, 20.7%, 20.8%, 20.9%, 21%, 21.1%, 21.2%, 21.3%, 21.4%, 21.5%, 21.6%, 21.7%, 21.8%, 21.9%, 22%, 22.1%, 22.2%, 22.3%, 22.4%, 22.5%, 22.6%, 22.7%, 22.8%, 22.9%, 23%, 23.1%, 23.2%, 23.3%, 23.4%, 23.5%, 23.6%, 23.7%, 23.8%, 23.9%, 24%, 24.1%, 24.2%, 24.3%, 24.4%, 24.5%, 24.6%, 24.7%, 24.8%, 24.9%, 25%, 25.1%, 25.2%, 25.3%, 25.4%, 25.5%, 25.6%, 25.7%, 25.8%, 25.9%, 26%, 26.1%, 26.2%, 26.3%, 26.4%, 26.5%, 26.6%, 26.7%, 26.8%, 26.9%, 27%, 27.1%, 27.2%, 27.3%, 27.4%, 27.5%, 27.6%, 27.7%, 27.8%, 27.9%, 28%, 28.1%, 28.2%, 28.3%, 28.4%, 28.5%, 28.6%, 28.7%, 28.8%, 28.9%, 29%, 29.1%, 29.2%, 29.3%, 29.4%, 29.5%, 29.6%, 29.7%, 29.8%, 29.9%, to/or 30.0% or more.

In some embodiments, the effective amount of the CLA improves hypogonadism or a symptom thereof. In some embodiments, the effective amount of the CLA improves testicular morphology and/or structure. In some embodiments, the effective amount of the CLA increases sperm maturation and/or number of mature sperm produced by the testes and/or in semen. In some embodiments, the effective amount of the CLA increases sperm maturation and/or the number of mature sperm produced by testes and/or in semen by 1% to 1,000% or more. In some embodiments, the effective amount of the CLA increases sperm maturation and/or the number of mature sperm produced by testes and/or in semen by about 1%, 5%, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, 200%, 205%, 210%, 215%, 220%, 225%, 230%, 235%, 240%, 245%, 250%, 255%, 260%, 265%, 270%, 275%, 280%, 285%, 290%, 295%, 300%, 305%, 310%, 315%, 320%, 325%, 330%, 335%, 340%, 345%, 350%, 355%, 360%, 365%, 370%, 375%, 380%, 385%, 390%, 395%, 400%, 405%, 410%, 415%, 420%, 425%, 430%, 435%, 440%, 445%, 450%, 455%, 460%, 465%, 470%, 475%, 480%, 485%, 490%, 495%, 500%, 505%, 510%, 515%, 520%, 525%, 530%, 535%, 540%, 545%, 550%, 555%, 560%, 565%, 570%, 575%, 580%, 585%, 590%, 595%, 600%, 605%, 610%, 615%, 620%, 625%, 630%, 635%, 640%, 645%, 650%, 655%, 660%, 665%, 670%, 675%, 680%, 685%, 690%, 695%, 700%, 705%, 710%, 715%, 720%, 725%, 730%, 735%, 740%, 745%, 750%, 755%, 760%, 765%, 770%, 775%, 780%, 785%, 790%, 795%, 800%, 805%, 810%, 815%, 820%, 825%, 830%, 835%, 840%, 845%, 850%, 855%, 860%, 865%, 870%, 875%, 880%, 885%, 890%, 895%, 900%, 905%, 910%, 915%, 920%, 925%, 930%, 935%, 940%, 945%, 950%, 955%, 960%, 965%, 970%, 975%, 980%, 985%, 990%, 995%, to/or 1,000% or more.

In some embodiments, the effective amount of CLA increases sperm differentiation. In some embodiments, the effective amount of CLA increases sperm differentiation by 1%-1,000% or more. In some embodiments, the effective amount of CLA increases sperm differentiation by about 1%, 5%, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, 200%, 205%, 210%, 215%, 220%, 225%, 230%, 235%, 240%, 245%, 250%, 255%, 260%, 265%, 270%, 275%, 280%, 285%, 290%, 295%, 300%, 305%, 310%, 315%, 320%, 325%, 330%, 335%, 340%, 345%, 350%, 355%, 360%, 365%, 370%, 375%, 380%, 385%, 390%, 395%, 400%, 405%, 410%, 415%, 420%, 425%, 430%, 435%, 440%, 445%, 450%, 455%, 460%, 465%, 470%, 475%, 480%, 485%, 490%, 495%, 500%, 505%, 510%, 515%, 520%, 525%, 530%, 535%, 540%, 545%, 550%, 555%, 560%, 565%, 570%, 575%, 580%, 585%, 590%, 595%, 600%, 605%, 610%, 615%, 620%, 625%, 630%, 635%, 640%, 645%, 650%, 655%, 660%, 665%, 670%, 675%, 680%, 685%, 690%, 695%, 700%, 705%, 710%, 715%, 720%, 725%, 730%, 735%, 740%, 745%, 750%, 755%, 760%, 765%, 770%, 775%, 780%, 785%, 790%, 795%, 800%, 805%, 810%, 815%, 820%, 825%, 830%, 835%, 840%, 845%, 850%, 855%, 860%, 865%, 870%, 875%, 880%, 885%, 890%, 895%, 900%, 905%, 910%, 915%, 920%, 925%, 930%, 935%, 940%, 945%, 950%, 955%, 960%, 965%, 970%, 975%, 980%, 985%, 990%, 995%, to/or 1,000% or more.

In some embodiments, the effective amount of CLA increases spermatogenesis. In some embodiments, the effective amount of CLA increases spermatogenesis about 1% to about 1,000% or more. In some embodiments, the effective amount of CLA increases spermatogenesis about 1%, 5%, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, 200%, 205%, 210%, 215%, 220%, 225%, 230%, 235%, 240%, 245%, 250%, 255%, 260%, 265%, 270%, 275%, 280%, 285%, 290%, 295%, 300%, 305%, 310%, 315%, 320%, 325%, 330%, 335%, 340%, 345%, 350%, 355%, 360%, 365%, 370%, 375%, 380%, 385%, 390%, 395%, 400%, 405%, 410%, 415%, 420%, 425%, 430%, 435%, 440%, 445%, 450%, 455%, 460%, 465%, 470%, 475%, 480%, 485%, 490%, 495%, 500%, 505%, 510%, 515%, 520%, 525%, 530%, 535%, 540%, 545%, 550%, 555%, 560%, 565%, 570%, 575%, 580%, 585%, 590%, 595%, 600%, 605%, 610%, 615%, 620%, 625%, 630%, 635%, 640%, 645%, 650%, 655%, 660%, 665%, 670%, 675%, 680%, 685%, 690%, 695%, 700%, 705%, 710%, 715%, 720%, 725%, 730%, 735%, 740%, 745%, 750%, 755%, 760%, 765%, 770%, 775%, 780%, 785%, 790%, 795%, 800%, 805%, 810%, 815%, 820%, 825%, 830%, 835%, 840%, 845%, 850%, 855%, 860%, 865%, 870%, 875%, 880%, 885%, 890%, 895%, 900%, 905%, 910%, 915%, 920%, 925%, 930%, 935%, 940%, 945%, 950%, 955%, 960%, 965%, 970%, 975%, 980%, 985%, 990%, 995%, to/or 1,000%.

In some embodiments, the effective amount of the CLA decreases testicular degeneration. In some embodiments, the effective amount of the CLA decreases testicular degeneration by about 1%-1,000% or more. In some embodiments, the effective amount of the CLA decreases testicular degeneration by about 1%, 5%, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, 200%, 205%, 210%, 215%, 220%, 225%, 230%, 235%, 240%, 245%, 250%, 255%, 260%, 265%, 270%, 275%, 280%, 285%, 290%, 295%, 300%, 305%, 310%, 315%, 320%, 325%, 330%, 335%, 340%, 345%, 350%, 355%, 360%, 365%, 370%, 375%, 380%, 385%, 390%, 395%, 400%, 405%, 410%, 415%, 420%, 425%, 430%, 435%, 440%, 445%, 450%, 455%, 460%, 465%, 470%, 475%, 480%, 485%, 490%, 495%, 500%, 505%, 510%, 515%, 520%, 525%, 530%, 535%, 540%, 545%, 550%, 555%, 560%, 565%, 570%, 575%, 580%, 585%, 590%, 595%, 600%, 605%, 610%, 615%, 620%, 625%, 630%, 635%, 640%, 645%, 650%, 655%, 660%, 665%, 670%, 675%, 680%, 685%, 690%, 695%, 700%, 705%, 710%, 715%, 720%, 725%, 730%, 735%, 740%, 745%, 750%, 755%, 760%, 765%, 770%, 775%, 780%, 785%, 790%, 795%, 800%, 805%, 810%, 815%, 820%, 825%, 830%, 835%, 840%, 845%, 850%, 855%, 860%, 865%, 870%, 875%, 880%, 885%, 890%, 895%, 900%, 905%, 910%, 915%, 920%, 925%, 930%, 935%, 940%, 945%, 950%, 955%, 960%, 965%, 970%, 975%, 980%, 985%, 990%, 995%, to/or 1,000%.

In certain example embodiments, the effective amount of CLA modulates (increases or decreases) expression, optionally the hypothalamic expression, of one or more genes. the In some embodiments, the effective amount of CLA modulates (increases or decreases) hypothalamic expression of one or more genes. In certain example embodiments, the effective amount of CLA modulates (increases or decreases) hypothalamic expression of one or more genes selected from: Ipw, Gria4, Ndm, Gm32061, Zswim5, Mael, Mfge8, Rertreg2, Mstol, Tecr, Zfp316, Spryd3, Mrps26, Hlf10, Eif6, Tmem59I, Serpina3n, Tubb2b, Slc12a2, Ppargcla, Dgkk, or any combination thereof. In certain example embodiments, the effective amount of CLA modulates (increases or decreases) expression, optionally the hypothalamic expression, of one or more genes selected from any one or more of those set forth in Supplementary Data tables S1-S3 of Knott et al., 2022. Nutrients. 14:860, which are incorporated by reference as if expressed in their entireties herein. In certain example embodiments, the effective amount of CLA modulates a biologic program or pathway, such as any of those set forth in any one of Tables 2-4.

In some embodiments, the effective amount of the CLA decreases anxiety or a symptom thereof. In some embodiments, the effective amount of the CLA decreases anxiety or a symptom thereof by about 1%-1,000% or more. In some embodiments, the effective amount of the CLA decreases anxiety or a symptom thereof by about 1%, 5%, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, 200%, 205%, 210%, 215%, 220%, 225%, 230%, 235%, 240%, 245%, 250%, 255%, 260%, 265%, 270%, 275%, 280%, 285%, 290%, 295%, 300%, 305%, 310%, 315%, 320%, 325%, 330%, 335%, 340%, 345%, 350%, 355%, 360%, 365%, 370%, 375%, 380%, 385%, 390%, 395%, 400%, 405%, 410%, 415%, 420%, 425%, 430%, 435%, 440%, 445%, 450%, 455%, 460%, 465%, 470%, 475%, 480%, 485%, 490%, 495%, 500%, 505%, 510%, 515%, 520%, 525%, 530%, 535%, 540%, 545%, 550%, 555%, 560%, 565%, 570%, 575%, 580%, 585%, 590%, 595%, 600%, 605%, 610%, 615%, 620%, 625%, 630%, 635%, 640%, 645%, 650%, 655%, 660%, 665%, 670%, 675%, 680%, 685%, 690%, 695%, 700%, 705%, 710%, 715%, 720%, 725%, 730%, 735%, 740%, 745%, 750%, 755%, 760%, 765%, 770%, 775%, 780%, 785%, 790%, 795%, 800%, 805%, 810%, 815%, 820%, 825%, 830%, 835%, 840%, 845%, 850%, 855%, 860%, 865%, 870%, 875%, 880%, 885%, 890%, 895%, 900%, 905%, 910%, 915%, 920%, 925%, 930%, 935%, 940%, 945%, 950%, 955%, 960%, 965%, 970%, 975%, 980%, 985%, 990%, 995%, to/or 1,000%.

In some embodiments, the effective amount of the CLA, modulates the α-diversity of the gut microbiome. As used in this context herein, α-diversity describes the structure of a microbial community in relation to the number of taxonomic groups and their respective abundance.

The CLA can be formulated for any suitable route of administration including, but not limited to, oral (including buccal or sublingual), intranasal, topical, parenteral, subcutaneous, intramuscular, intravenous, internasal, and intradermal. Such formulations can be prepared by any method known in the art.

In certain example embodiments, the dietary supplement, feed, or foodstuff or beverage formulation is a liquid, solid, semi-solid, or an emulsion.

In some embodiments, the dietary supplement, feed, or foodstuff or beverage formulation is a powder or contains a powder. In some embodiments, the powder is formulated to be reconstituted or diluted in a liquid or emulsion. In some embodiments, the beverage formulation is a syrup, drink or sake, or other liquid formulated for oral consumption.

Dosage forms (such as the dietary supplement, feed, foodstuffs or beverage) formulated for oral administration can discrete dosage units such as capsules, pellets or tablets, powders or granules, solutions, or suspensions in aqueous or non-aqueous liquids; edible foams or whips, or in oil-in-water liquid emulsions or water-in-oil liquid emulsions. In some embodiments, the pharmaceutical formulations adapted for oral administration also include one or more agents which flavor, preserve, color, or help disperse the formulation. Dosage forms prepared for oral administration can also be in the form of a liquid solution that can be delivered as a foam, spray, or liquid solution (e.g., shake or other composition). The oral dosage form can be administered to a subject in need thereof. Exemplary configurations and methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20^{th} ed., Lippincott Williams & Wilkins, Baltimore, M D, 2000, and “Pharmaceutical dosage forms and drug delivery systems”, 6^{th} Edition, Ansel et al., (Media, PA: Williams and Wilkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

In some embodiments, the CLA formulation can be formulated as a delayed release formulation in which the CLA will be released over a period of time from hours, days, weeks, or months.

Coatings may be formed with a different ratio of water-soluble polymer, water insoluble polymers, and/or pH dependent polymers, with or without water insoluble/water soluble non-polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Where appropriate, the dosage forms described herein can be a liposome. In these embodiments, primary active ingredient(s), and/or optional secondary active ingredient(s), and/or pharmaceutically acceptable salt thereof where appropriate are incorporated into a liposome. In embodiments where the dosage form is a liposome, the pharmaceutical formulation is thus a liposomal formulation. The liposomal formulation can be administered to a subject in need thereof.

Dosage forms adapted for topical administration can be formulated as ointments, creams, suspensions, lotions, powders, solutions, pastes, gels, sprays, aerosols, or oils. In some embodiments for treatments of the eye or other external tissues, for example the mouth or the skin, the pharmaceutical formulations are applied as a topical ointment or cream. When formulated in an ointment, a primary active ingredient, optional secondary active ingredient, and/or pharmaceutically acceptable salt thereof where appropriate can be formulated with a paraffinic or water-miscible ointment base. In other embodiments, the primary and/or secondary active ingredient can be formulated in a cream with an oil-in-water cream base or a water-in-oil base. Dosage forms adapted for topical administration in the mouth include lozenges, pastilles, and mouth washes.

Dosage forms adapted for nasal or inhalation administration include aerosols, solutions, suspension drops, gels, or dry powders. In some embodiments, a primary active ingredient, optional secondary active ingredient, and/or pharmaceutically acceptable salt thereof where appropriate can be in a dosage form adapted for inhalation is in a particle-size-reduced form that is obtained or obtainable by micronization. In some embodiments, the particle size of the size reduced (e.g., micronized) compound or salt or solvate thereof, is defined by a D50 value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active (primary and/or secondary) ingredient, which may be generated by various types of metered dose pressurized aerosols, nebulizers, or insufflators. The nasal/inhalation formulations can be administered to a subject in need thereof.

In some embodiments, the dosage forms are aerosol formulations suitable for administration by inhalation. In some of these embodiments, the aerosol formulation contains a solution or fine suspension of a primary active ingredient, secondary active ingredient, and/or pharmaceutically acceptable salt thereof where appropriate and a pharmaceutically acceptable aqueous or non-aqueous solvent. Aerosol formulations can be presented in single or multi-dose quantities in sterile form in a sealed container. For some of these embodiments, the sealed container is a single dose or multi-dose nasal or an aerosol dispenser fitted with a metering valve (e.g. metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of a primary active ingredient, optional secondary active ingredient, and/or pharmaceutically acceptable salt thereof. In further embodiments, the aerosol formulation also contains co-solvents and/or modifiers incorporated to improve, for example, the stability and/or taste and/or fine particle mass characteristics (amount and/or profile) of the formulation. Administration of the aerosol formulation can be once daily or several times daily, for example 2, 3, 4, or 8 times daily, in which 1, 2, 3 or more doses are delivered each time. The aerosol formulations can be administered to a subject in need thereof.

For some dosage forms suitable and/or adapted for inhaled administration, the pharmaceutical formulation is a dry powder inhalable-formulations. In addition to a primary active agent, optional secondary active ingredient, and/or pharmaceutically acceptable salt thereof where appropriate, such a dosage form can contain a powder base such as lactose, glucose, trehalose, manitol, and/or starch. In some of these embodiments, a primary active agent, secondary active ingredient, and/or pharmaceutically acceptable salt thereof where appropriate is in a particle-size reduced form. In further embodiments, a performance modifier, such as L-leucine or another amino acid, cellobiose octaacetate, and/or metals salts of stearic acid, such as magnesium or calcium stearate. In some embodiments, the aerosol formulations are arranged so that each metered dose of aerosol contains a predetermined amount of an active ingredient, such as the one or more of the compositions, compounds, vector(s), molecules, cells, and combinations thereof described herein.

Dosage forms adapted for parenteral administration and/or adapted for injection can include aqueous and/or non-aqueous sterile injection solutions, which can contain antioxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and re-suspended in a sterile carrier to reconstitute the dose prior to administration. Extemporaneous injection solutions and suspensions can be prepared in some embodiments, from sterile powders, granules, and tablets. The parenteral formulations can be administered to a subject in need thereof.

For some embodiments, the dosage form contains a predetermined amount of the CLA or formulation thereof. In some embodiments, the predetermined amount of the CLA in a dosage form is less than the effective amount of the CLA. In these such embodiments, the subject can take multiple dosage forms to add up to the effective amount of the CLA. For example, if the effective amount of CLA is 3,000 mg per day, this can be divided into e.g., two dosage forms each containing 1500 mg of CLA and formulated such that the subject in need thereof is administered the two dosage forms per day to reach the effective amount of 3,0000 mg CLA per day. One of ordinary skill in the art will envision different dosages containing predetermined amounts of CLA in view of the disclosure provided herein.

In some embodiments, the effective amount or dosage form of the CLA is contained in a feed formulation, additive, or feed supplement formulated for administration to a non-human animal. In some embodiments, the non-human animal is a bovine, equine, caprine, swine, ovine, avian, fish and/or the like. In some embodiments, the formulation is a feed formulation that is formulated as a complete diet. In some embodiments, the formulation is a feed supplement. In some embodiments, the formulation is a feed additive. As used herein, “complete diet” refers to a feed formulation that contains all the nutrients, calories, minerals, vitamins, and other components needed to meet the dietary requirements of an animal without any additional feed sources and intended to be fed as the entire food source fir the animal. As used herein, “feed additive” is an extra nutrient or non-nutrient component that is provided or can be added to a feed or diet that is beyond or in addition to the basic nutritional components of a feed or diet. Feed additives generally fall or can include components that fall into five basic categories: technological additives (e.g., preservatives, antioxidants, emulsifiers, stabilizing agents, acidity regulators, binding agents, silage additives, and/or the like), sensory additives (e.g., flavors, colorants, and/or the like), nutritional additives (e.g., vitamins, minerals, amino acids, trace elements and other minerals, and/or the like), zootechnical additives (e.g., digestibility enhancers, enzymes, intestinal health enhancers, microbes, fiber, and/or the like), and pharmaceuticals/nutraceuticals. Technological additives are substances or compositions that serves a technological purpose in a feed formulation. Sensory additives are substances or compositions that improve or otherwise changes the organoleptic properties of the feed, or the visual characteristics of the food or other product derived from animals. Nutritional additives are any nutrient substance or component. Zootehnical additives are substances and compositions that improve, favorably affect, or otherwise modify the health and/or performance of an animal and/or modify their impact on the environment. It will be appreciated that any one particular feed additive or component can fall into more than one category. Feed additives can be added to a feed formulation at any stage during feed formulation production (such as being provided in or as a pre-mix) or provided to an animal as a feed supplement that is separate from the feed formulation and added at the point of feeding. As used herein, “feed supplement” refers to a composition formulated for consumption by an animal and intended to be fed undiluted as a supplement to other feeds or offered free choice with other parts of the ration separately available or further diluted and mixed to produce a complete feed ration. A feed additive are intended to be fully incorporated into a feed formulation, while feed supplements indented to be stand-alone compositions that can be fed free choice or mixed in with a feed ration at point of feeding or can be mixed into a feed formulation to make a complete feed formulation. A feed supplement can include one or more feed additive(s). In some embodiments, the feed formulation is formulated to meet the nutritional requirements of a specific age or life-stage or provide some other benefit that is specific to age or life stage. In some embodiments, the feed formulation is formulated to support or meet specific requirements of an animal in a diseased or otherwise non-healthy or normal state. In some embodiments, the feed formulation is formulated to enhance or support the performance of an animal.

In certain example embodiments, the dietary supplement, feed, or foodstuff or beverage formulation or other CLA formulation described herein is adapted for daily, every other day, weekly, or monthly administration. In certain example embodiments, the effective amount of CLA or formulation thereof is a liquid, solid, semi-solid, or an emulsion. Other formulations and dosage forms are described elsewhere herein. In certain example embodiments, the effective amount of CLA or formulation thereof is a dietary supplement or feed or foodstuff or beverage formulation. Exemplary is a dietary supplement or feed or foodstuff or beverage formulations and/or the like are described in greater detail elsewhere herein

In certain example embodiments, the subject in need thereof is a mammal, optionally a human.

In certain example embodiments, the dietary supplement, feed, or foodstuff or beverage formulation is adapted for daily, every other day, weekly, or monthly administration. In some embodiments, the dietary supplement the dietary supplement, feed, or foodstuff or beverage formulation is adapted for administration 1, 2, or 3 times daily. In some embodiments, such dosage forms are formulated such that an effective amount is delivered in 1, 2 or 3 amounts, where in the case of 2 or 3 doses, the total effective amount is split between the 2 or 3 doses.

### Kits

Any of the compounds, compositions, or formulations described herein or a combination thereof can be presented as a combination kit. As used herein, the terms “combination kit” or “kit of parts” refers to the compounds, compositions, or formulations and any additional components that are used to package, sell, market, deliver, and/or administer the combination of elements or a single element, such as the active ingredient, contained therein. Such additional components include, but are not limited to, packaging, syringes, blister packages, bottles, and the like. When one or more of the compounds, compositions, or formulations described herein or a combination thereof (e.g., agents) contained in the kit are administered simultaneously, the combination kit can contain the active agents in a single formulation, such as a pharmaceutical formulation, (e.g., a tablet) or in separate formulations. When the compounds, compositions or formulations described herein or a combination thereof and/or kit components are not administered simultaneously, the combination kit can contain each agent or other component in separate pharmaceutical formulations. The separate kit components can be contained in a single package or in separate packages within the kit.

In some embodiments, the combination kit also includes instructions printed on or otherwise contained in a tangible medium of expression. The instructions can provide information regarding the content of the compounds, compositions, or formulations described herein or a combination thereof contained therein, safety information regarding the content of the compounds, compositions, or formulations (e.g., nutraceutical formulations) described herein or a combination thereof contained therein, information regarding the dosages, indications for use, and/or recommended treatment regimen(s) for the compound(s) and/or formulations contained therein. In some embodiments, the instructions can provide directions for administering the compounds, compositions, or formulations described herein or a combination thereof to a subject in need thereof. In some embodiments, the subject in need thereof is in need of a treatment or prevention for a Snord116 deficiency, optionally PWS, or a symptom thereof. In some embodiments, the subject in need thereof is in need of a treatment or prevention for hypogonadism or a symptom thereof in a subject having a Nhlh2 deficiency, disease, or symptom thereof.

Methods of Treating Disease and Symptoms Thereof with CLA

Described in certain embodiments herein are method of treating a Snord116 deficiency, disease, or a symptom thereof in a subject in need thereof that includes administering a CLA composition or formulation thereof described elsewhere herein to the subject in need thereof. In some embodiments, the Snord116 deficiency disease is Prader-Willi Syndrome (PWS). Described in certain embodiments herein are methods of treating hypogonadism in a subject having a Nhlh2 deficiency, disease, or symptom thereof, the method comprising administering a CLA composition or formulation thereof described in greater detail elsewhere herein to the subject in need thereof.

Described in certain embodiments herein are methods of treating a Snord116 deficiency, disease, or a symptom thereof in a subject in need thereof, the method comprising administering an effective amount of conjugated linoleic acid (CLA) or a formulation thereof to the subject in need thereof, optionally wherein the effective amount is administered in one or more doses. In certain example embodiments, the Snord116 deficiency disease is Prader-Willi Syndrome (PWS).

Described in certain example embodiments herein are methods of treating hypogonadism in a subject having a Nhlh2 deficiency, disease, or symptom thereof, the method comprising an effective amount of conjugated linoleic acid (CLA) or a formulation thereof to the subject in need thereof.

In certain example embodiments, the effective amount of CLA is at least 1,000 mg per day, optionally about 3,000 to about 5,000 mg per day. In certain example embodiments, the effective amount of the CLA is about 3-10 g/kg body weight per day, optionally about 5 g/kg body weight per day. In certain example embodiments, the CLA or formulation thereof contains or is composed entirely of a mixture of CLA isoforms, optionally a 50:50 mixture of trans-10, cis-12 and trans9, cis-11 CLA isomers. In certain example embodiments, the CLA or formulation thereof contains 80-100% of a mixture of CLA isoforms, optionally a 50:50 mixture of trans-10, cis-12 and trans9, cis-11 CLA isomers.

In certain example embodiments, the effective amount of CLA is at least 1,000 mg per day, optionally about 3,000 to about 5,000 mg per day. In certain example embodiments, the effective amount of CLA is about 1000 mg, 1025 mg, 1050 mg, 1075 mg, 1100 mg, 1125 mg, 1150 mg, 1175 mg, 1200 mg, 1225 mg, 1250 mg, 1275 mg, 1300 mg, 1325 mg, 1350 mg, 1375 mg, 1400 mg, 1425 mg, 1450 mg, 1475 mg, 1500 mg, 1525 mg, 1550 mg, 1575 mg, 1600 mg, 1625 mg, 1650 mg, 1675 mg, 1700 mg, 1725 mg, 1750 mg, 1775 mg, 1800 mg, 1825 mg, 1850 mg, 1875 mg, 1900 mg, 1925 mg, 1950 mg, 1975 mg, 2000 mg, 2025 mg, 2050 mg, 2075 mg, 2100 mg, 2125 mg, 2150 mg, 2175 mg, 2200 mg, 2225 mg, 2250 mg, 2275 mg, 2300 mg, 2325 mg, 2350 mg, 2375 mg, 2400 mg, 2425 mg, 2450 mg, 2475 mg, 2500 mg, 2525 mg, 2550 mg, 2575 mg, 2600 mg, 2625 mg, 2650 mg, 2675 mg, 2700 mg, 2725 mg, 2750 mg, 2775 mg, 2800 mg, 2825 mg, 2850 mg, 2875 mg, 2900 mg, 2925 mg, 2950 mg, 2975 mg, 3000 mg, 3025 mg, 3050 mg, 3075 mg, 3100 mg, 3125 mg, 3150 mg, 3175 mg, 3200 mg, 3225 mg, 3250 mg, 3275 mg, 3300 mg, 3325 mg, 3350 mg, 3375 mg, 3400 mg, 3425 mg, 3450 mg, 3475 mg, 3500 mg, 3525 mg, 3550 mg, 3575 mg, 3600 mg, 3625 mg, 3650 mg, 3675 mg, 3700 mg, 3725 mg, 3750 mg, 3775 mg, 3800 mg, 3825 mg, 3850 mg, 3875 mg, 3900 mg, 3925 mg, 3950 mg, 3975 mg, 4000 mg, 4025 mg, 4050 mg, 4075 mg, 4100 mg, 4125 mg, 4150 mg, 4175 mg, 4200 mg, 4225 mg, 4250 mg, 4275 mg, 4300 mg, 4325 mg, 4350 mg, 4375 mg, 4400 mg, 4425 mg, 4450 mg, 4475 mg, 4500 mg, 4525 mg, 4550 mg, 4575 mg, 4600 mg, 4625 mg, 4650 mg, 4675 mg, 4700 mg, 4725 mg, 4750 mg, 4775 mg, 4800 mg, 4825 mg, 4850 mg, 4875 mg, 4900 mg, 4925 mg, 4950 mg, 4975 mg, 5000 mg, 5025 mg, 5050 mg, 5075 mg, 5100 mg, 5125 mg, 5150 mg, 5175 mg, 5200 mg, 5225 mg, 5250 mg, 5275 mg, 5300 mg, 5325 mg, 5350 mg, 5375 mg, 5400 mg, 5425 mg, 5450 mg, 5475 mg, 5500 mg, 5525 mg, 5550 mg, 5575 mg, 5600 mg, 5625 mg, 5650 mg, 5675 mg, 5700 mg, 5725 mg, 5750 mg, 5775 mg, 5800 mg, 5825 mg, 5850 mg, 5875 mg, 5900 mg, 5925 mg, 5950 mg, 5975 mg, 6000 mg, 6025 mg, 6050 mg, 6075 mg, 6100 mg, 6125 mg, 6150 mg, 6175 mg, 6200 mg, 6225 mg, 6250 mg, 6275 mg, 6300 mg, 6325 mg, 6350 mg, 6375 mg, 6400 mg, 6425 mg, 6450 mg, 6475 mg, 6500 mg, 6525 mg, 6550 mg, 6575 mg, 6600 mg, 6625 mg, 6650 mg, 6675 mg, 6700 mg, 6725 mg, 6750 mg, 6775 mg, 6800 mg, 6825 mg, 6850 mg, 6875 mg, 6900 mg, 6925 mg, 6950 mg, 6975 mg, 7000 mg, 7025 mg, 7050 mg, 7075 mg, 7100 mg, 7125 mg, 7150 mg, 7175 mg, 7200 mg, 7225 mg, 7250 mg, 7275 mg, 7300 mg, 7325 mg, 7350 mg, 7375 mg, 7400 mg, 7425 mg, 7450 mg, 7475 mg, 7500 mg, 7525 mg, 7550 mg. 7575 mg, 7600 mg, 7625 mg, 7650 mg, 7675 mg, 7700 mg, 7725 mg, 7750 mg, 7775 mg, 7800 mg, 7825 mg, 7850 mg, 7875 mg, 7900 mg, 7925 mg, 7950 mg, 7975 mg, 8000 mg, 8025 mg, 8050 mg, 8075 mg, 8100 mg, 8125 mg, 8150 mg, 8175 mg, 8200 mg, 8225 mg, 8250 mg, 8275 mg, 8300 mg, 8325 mg, 8350 mg, 8375 mg, 8400 mg, 8425 mg, 8450 mg, 8475 mg, 8500 mg, 8525 mg, 8550 mg, 8575 mg, 8600 mg, 8625 mg, 8650 mg, 8675 mg, 8700 mg, 8725 mg, 8750 mg, 8775 mg, 8800 mg, 8825 mg, 8850 mg, 8875 mg, 8900 mg, 8925 mg, 8950 mg, 8975 mg, 9000 mg, 9025 mg, 9050 mg, 9075 mg, 9100 mg, 9125 mg, 9150 mg, 9175 mg, 9200 mg, 9225 mg, 9250 mg, 9275 mg, 9300 mg, 9325 mg, 9350 mg, 9375 mg, 9400 mg, 9425 mg, 9450 mg, 9475 mg, 9500 mg, 9525 mg, 9550 mg, 9575 mg, 9600 mg, 9625 mg, 9650 mg, 9675 mg, 9700 mg, 9725 mg, 9750 mg, 9775 mg, 9800 mg, 9825 mg, 9850 mg, 9875 mg, 9900 mg, 9925 mg, 9950 mg, 9975 mg, to/or 10000 mg. In certain example embodiments, the effective amount of CLA is about 3000 mg, 3025 mg, 3050 mg, 3075 mg, 3100 mg, 3125 mg, 3150 mg, 3175 mg, 3200 mg, 3225 mg, 3250 mg, 3275 mg, 3300 mg, 3325 mg, 3350 mg, 3375 mg, 3400 mg, 3425 mg, 3450 mg, 3475 mg, 3500 mg, 3525 mg, 3550 mg, 3575 mg, 3600 mg, 3625 mg, 3650 mg, 3675 mg, 3700 mg, 3725 mg, 3750 mg, 3775 mg, 3800 mg, 3825 mg, 3850 mg, 3875 mg, 3900 mg, 3925 mg, 3950 mg, 3975 mg, 4000 mg, 4025 mg, 4050 mg, 4075 mg, 4100 mg, 4125 mg, 4150 mg, 4175 mg, 4200 mg, 4225 mg, 4250 mg, 4275 mg, 4300 mg, 4325 mg, 4350 mg, 4375 mg, 4400 mg, 4425 mg, 4450 mg, 4475 mg, 4500 mg, 4525 mg, 4550 mg, 4575 mg, 4600 mg, 4625 mg, 4650 mg, 4675 mg, 4700 mg, 4725 mg, 4750 mg, 4775 mg, 4800 mg, 4825 mg, 4850 mg, 4875 mg, 4900 mg, 4925 mg, 4950 mg, 4975 mg, to/or about 5000 mg. In certain example embodiments, the effective amount of CLA is about 3000 mg.

In certain example embodiments, the effective amount of the CLA is about 3-10 g/kg body weight per day, optionally about 5 g/kg body weight per day. In certain example embodiments, the effective amount of the CLA is about 3 g/kg body weight, 3.1 g/kg body weight, 3.2 g/kg body weight, 3.3 g/kg body weight, 3.4 g/kg body weight, 3.5 g/kg body weight, 3.6 g/kg body weight, 3.7 g/kg body weight, 3.8 g/kg body weight, 3.9 g/kg body weight, 4 g/kg body weight, 4.1 g/kg body weight, 4.2 g/kg body weight, 4.3 g/kg body weight, 4.4 g/kg body weight, 4.5 g/kg body weight, 4.6 g/kg body weight, 4.7 g/kg body weight, 4.8 g/kg body weight, 4.9 g/kg body weight, 5 g/kg body weight, 5.1 g/kg body weight, 5.2 g/kg body weight, 5.3 g/kg body weight, 5.4 g/kg body weight, 5.5 g/kg body weight, 5.6 g/kg body weight, 5.7 g/kg body weight, 5.8 g/kg body weight, 5.9 g/kg body weight, 6 g/kg body weight, 6.1 g/kg body weight, 6.2 g/kg body weight, 6.3 g/kg body weight, 6.4 g/kg body weight, 6.5 g/kg body weight, 6.6 g/kg body weight, 6.7 g/kg body weight, 6.8 g/kg body weight, 6.9 g/kg body weight, 7 g/kg body weight, 7.1 g/kg body weight, 7.2 g/kg body weight, 7.3 g/kg body weight, 7.4 g/kg body weight, 7.5 g/kg body weight, 7.6 g/kg body weight, 7.7 g/kg body weight, 7.8 g/kg body weight, 7.9 g/kg body weight, 8 g/kg body weight, 8.1 g/kg body weight, 8.2 g/kg body weight, 8.3 g/kg body weight, 8.4 g/kg body weight, 8.5 g/kg body weight, 8.6 g/kg body weight, 8.7 g/kg body weight, 8.8 g/kg body weight, 8.9 g/kg body weight, 9 g/kg body weight, 9.1 g/kg body weight, 9.2 g/kg body weight, 9.3 g/kg body weight, 9.4 g/kg body weight, 9.5 g/kg body weight, 9.6 g/kg body weight, 9.7 g/kg body weight, 9.8 g/kg body weight, 9.9 g/kg body weight, to/or 10 g/kg body weight. In certain example embodiments, the effective amount of the CLA is about 4.5 g/kg body weight, 4.6 g/kg body weight, 4.7 g/kg body weight, 4.8 g/kg body weight, 4.9 g/kg body weight, 5 g/kg body weight, 5.1 g/kg body weight, 5.2 g/kg body weight, 5.3 g/kg body weight, 5.4 g/kg body weight, to/or about 5.5 g/kg body weight. In certain example embodiments, the effective amount of the CLA is about 5.0 g/kg body weight, 5.1 g/kg body weight, to/or 5.2 g/kg body weight.

In certain example embodiments, the CLA or formulation thereof contains or is composed entirely of a mixture of CLA isoforms, optionally a 50:50 mixture of trans-10, cis-12 and trans9, cis-11 CLA isomers. In certain example embodiments, the CLA or formulation thereof contains 80-100% of a mixture of CLA isoforms. In some embodiments, the mixture of CLA isoforms is a 50:50 mixture of trans-10, cis-12 and trans9, cis-11 CLA isomers. In some embodiments, the CLA or formulation thereof contains about 80%, 81%, 82%, 83%, 84%, 85%, 86%, 87%, 88%, 89%, 90%, 91%, 92%, 93%, 94%, 95%, 96%, 97%, 98%, 99%, to/or 100%, of a mixture of CLA isoforms. In some embodiments, the mixture of CLA isoforms is a 50:50 mixture of trans-10, cis-12 and trans9, cis-11 CLA isomers. In some embodiments, the CLA or formulation thereof is Tonalin™ CLA.

In certain example embodiments, the effective amount of CLA decreases body weight, decreases fat mass, increases lean body mass, improves hypogonadism or a symptom thereof, improves testicular morphology, modulates the α-diversity of the gut microbiome, modulates hypothalamic gene expression of one or more genes, decreases anxiety, increase sperm maturation, increase spermatogenesis, increase sperm differentiation, decrease testicular degeneration or any combination thereof in the subject in need thereof.

In certain example embodiments, the effective amount of CLA decreases body weight, decreases fat mass, increases lean body mass improves hypogonadism or a symptom thereof, improves testicular morphology, increases sperm maturation, increase spermatogenesis, increase sperm differentiation, decrease testicular degeneration or any combination thereof in the subject in need thereof.

In some embodiments, the effective amount of CLA decreases body weight by about 0.1% to about 30% or more. In some embodiments, the effective amount of CLA decreases body weight by about 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 5.1%, 5.2%, 5.3%, 5.4%, 5.5%, 5.6%, 5.7%, 5.8%, 5.9%, 6%, 6.1%, 6.2%, 6.3%, 6.4%, 6.5%, 6.6%, 6.7%, 6.8%, 6.9%, 7%, 7.1%, 7.2%, 7.3%, 7.4%, 7.5%, 7.6%, 7.7%, 7.8%, 7.9%, 8%, 8.1%, 8.2%, 8.3%, 8.4%, 8.5%, 8.6%, 8.7%, 8.8%, 8.9%, 9%, 9.1%, 9.2%, 9.3%, 9.4%, 9.5%, 9.6%, 9.7%, 9.8%, 9.9%, 10%, 10.1%, 10.2%, 10.3%, 10.4%, 10.5%, 10.6%, 10.7%, 10.8%, 10.9%, 11%, 11.1%, 11.2%, 11.3%, 11.4%, 11.5%, 11.6%, 11.7%, 11.8%, 11.9%, 12%, 12.1%, 12.2%, 12.3%, 12.4%, 12.5%, 12.6%, 12.7%, 12.8%, 12.9%, 13%, 13.1%, 13.2%, 13.3%, 13.4%, 13.5%, 13.6%, 13.7%, 13.8%, 13.9%, 14%, 14.1%, 14.2%, 14.3%, 14.4%, 14.5%, 14.6%, 14.7%, 14.8%, 14.9%, 15%, 15.1%, 15.2%, 15.3%, 15.4%, 15.5%, 15.6%, 15.7%, 15.8%, 15.9%, 16%, 16.1%, 16.2%, 16.3%, 16.4%, 16.5%, 16.6%, 16.7%, 16.8%, 16.9%, 17%, 17.1%, 17.2%, 17.3%, 17.4%, 17.5%, 17.6%, 17.7%, 17.8%, 17.9%, 18%, 18.1%, 18.2%, 18.3%, 18.4%, 18.5%, 18.6%, 18.7%, 18.8%, 18.9%, 19%, 19.1%, 19.2%, 19.3%, 19.4%, 19.5%, 19.6%, 19.7%, 19.8%, 19.9%, 20%, 20.1%, 20.2%, 20.3%, 20.4%, 20.5%, 20.6%, 20.7%, 20.8%, 20.9%, 21%, 21.1%, 21.2%, 21.3%, 21.4%, 21.5%, 21.6%, 21.7%, 21.8%, 21.9%, 22%, 22.1%, 22.2%, 22.3%, 22.4%, 22.5%, 22.6%, 22.7%, 22.8%, 22.9%, 23%, 23.1%, 23.2%, 23.3%, 23.4%, 23.5%, 23.6%, 23.7%, 23.8%, 23.9%, 24%, 24.1%, 24.2%, 24.3%, 24.4%, 24.5%, 24.6%, 24.7%, 24.8%, 24.9%, 25%, 25.1%, 25.2%, 25.3%, 25.4%, 25.5%, 25.6%, 25.7%, 25.8%, 25.9%, 26%, 26.1%, 26.2%, 26.3%, 26.4%, 26.5%, 26.6%, 26.7%, 26.8%, 26.9%, 27%, 27.1%, 27.2%, 27.3%, 27.4%, 27.5%, 27.6%, 27.7%, 27.8%, 27.9%, 28%, 28.1%, 28.2%, 28.3%, 28.4%, 28.5%, 28.6%, 28.7%, 28.8%, 28.9%, 29%, 29.1%, 29.2%, 29.3%, 29.4%, 29.5%, 29.6%, 29.7%, 29.8%, 29.9%, to/or 30.0% or more.

In some embodiments, the effective amount of CLA decreases fat mass by about 0.1% to about 30% or more. In some embodiments, the effective amount of CLA decreases fat mass by about 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 5.1%, 5.2%, 5.3%, 5.4%, 5.5%, 5.6%, 5.7%, 5.8%, 5.9%, 6%, 6.1%, 6.2%, 6.3%, 6.4%, 6.5%, 6.6%, 6.7%, 6.8%, 6.9%, 7%, 7.1%, 7.2%, 7.3%, 7.4%, 7.5%, 7.6%, 7.7%, 7.8%, 7.9%, 8%, 8.1%, 8.2%, 8.3%, 8.4%, 8.5%, 8.6%, 8.7%, 8.8%, 8.9%, 9%, 9.1%, 9.2%, 9.3%, 9.4%, 9.5%, 9.6%, 9.7%, 9.8%, 9.9%, 10%, 10.1%, 10.2%, 10.3%, 10.4%, 10.5%, 10.6%, 10.7%, 10.8%, 10.9%, 11%, 11.1%, 11.2%, 11.3%, 11.4%, 11.5%, 11.6%, 11.7%, 11.8%, 11.9%, 12%, 12.1%, 12.2%, 12.3%, 12.4%, 12.5%, 12.6%, 12.7%, 12.8%, 12.9%, 13%, 13.1%, 13.2%, 13.3%, 13.4%, 13.5%, 13.6%, 13.7%, 13.8%, 13.9%, 14%, 14.1%, 14.2%, 14.3%, 14.4%, 14.5%, 14.6%, 14.7%, 14.8%, 14.9%, 15%, 15.1%, 15.2%, 15.3%, 15.4%, 15.5%, 15.6%, 15.7%, 15.8%, 15.9%, 16%, 16.1%, 16.2%, 16.3%, 16.4%, 16.5%, 16.6%, 16.7%, 16.8%, 16.9%, 17%, 17.1%, 17.2%, 17.3%, 17.4%, 17.5%, 17.6%, 17.7%, 17.8%, 17.9%, 18%, 18.1%, 18.2%, 18.3%, 18.4%, 18.5%, 18.6%, 18.7%, 18.8%, 18.9%, 19%, 19.1%, 19.2%, 19.3%, 19.4%, 19.5%, 19.6%, 19.7%, 19.8%, 19.9%, 20%, 20.1%, 20.2%, 20.3%, 20.4%, 20.5%, 20.6%, 20.7%, 20.8%, 20.9%, 21%, 21.1%, 21.2%, 21.3%, 21.4%, 21.5%, 21.6%, 21.7%, 21.8%, 21.9%, 22%, 22.1%, 22.2%, 22.3%, 22.4%, 22.5%, 22.6%, 22.7%, 22.8%, 22.9%, 23%, 23.1%, 23.2%, 23.3%, 23.4%, 23.5%, 23.6%, 23.7%, 23.8%, 23.9%, 24%, 24.1%, 24.2%, 24.3%, 24.4%, 24.5%, 24.6%, 24.7%, 24.8%, 24.9%, 25%, 25.1%, 25.2%, 25.3%, 25.4%, 25.5%, 25.6%, 25.7%, 25.8%, 25.9%, 26%, 26.1%, 26.2%, 26.3%, 26.4%, 26.5%, 26.6%, 26.7%, 26.8%, 26.9%, 27%, 27.1%, 27.2%, 27.3%, 27.4%, 27.5%, 27.6%, 27.7%, 27.8%, 27.9%, 28%, 28.1%, 28.2%, 28.3%, 28.4%, 28.5%, 28.6%, 28.7%, 28.8%, 28.9%, 29%, 29.1%, 29.2%, 29.3%, 29.4%, 29.5%, 29.6%, 29.7%, 29.8%, 29.9%, to/or 30.0% or more.

In some embodiments, the effective amount of the CLA improves hypogonadism or a symptom thereof. In some embodiments, the effective amount of the CLA improves testicular morphology and/or structure. In some embodiments, the effective amount of the CLA increases sperm maturation and/or number of mature sperm produced by the testes and/or in semen. In some embodiments, the effective amount of the CLA increases sperm maturation and/or the number of mature sperm produced by testes and/or in semen by 1% to 1,000% or more. In some embodiments, the effective amount of the CLA increases sperm maturation and/or the number of mature sperm produced by testes and/or in semen by about 1%, 5%, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, 200%, 205%, 210%, 215%, 220%, 225%, 230%, 235%, 240%, 245%, 250%, 255%, 260%, 265%, 270%, 275%, 280%, 285%, 290%, 295%, 300%, 305%, 310%, 315%, 320%, 325%, 330%, 335%, 340%, 345%, 350%, 355%, 360%, 365%, 370%, 375%, 380%, 385%, 390%, 395%, 400%, 405%, 410%, 415%, 420%, 425%, 430%, 435%, 440%, 445%, 450%, 455%, 460%, 465%, 470%, 475%, 480%, 485%, 490%, 495%, 500%, 505%, 510%, 515%, 520%, 525%, 530%, 535%, 540%, 545%, 550%, 555%, 560%, 565%, 570%, 575%, 580%, 585%, 590%, 595%, 600%, 605%, 610%, 615%, 620%, 625%, 630%, 635%, 640%, 645%, 650%, 655%, 660%, 665%, 670%, 675%, 680%, 685%, 690%, 695%, 700%, 705%, 710%, 715%, 720%, 725%, 730%, 735%, 740%, 745%, 750%, 755%, 760%, 765%, 770%, 775%, 780%, 785%, 790%, 795%, 800%, 805%, 810%, 815%, 820%, 825%, 830%, 835%, 840%, 845%, 850%, 855%, 860%, 865%, 870%, 875%, 880%, 885%, 890%, 895%, 900%, 905%, 910%, 915%, 920%, 925%, 930%, 935%, 940%, 945%, 950%, 955%, 960%, 965%, 970%, 975%, 980%, 985%, 990%, 995%, to/or 1,000% or more.

In some embodiments, the effective amount of CLA increases sperm differentiation. In some embodiments, the effective amount of CLA increases sperm differentiation by 1%-1,000% or more. In some embodiments, the effective amount of CLA increases sperm differentiation by about 1%, 5%, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, 200%, 205%, 210%, 215%, 220%, 225%, 230%, 235%, 240%, 245%, 250%, 255%, 260%, 265%, 270%, 275%, 280%, 285%, 290%, 295%, 300%, 305%, 310%, 315%, 320%, 325%, 330%, 335%, 340%, 345%, 350%, 355%, 360%, 365%, 370%, 375%, 380%, 385%, 390%, 395%, 400%, 405%, 410%, 415%, 420%, 425%, 430%, 435%, 440%, 445%, 450%, 455%, 460%, 465%, 470%, 475%, 480%, 485%, 490%, 495%, 500%, 505%, 510%, 515%, 520%, 525%, 530%, 535%, 540%, 545%, 550%, 555%, 560%, 565%, 570%, 575%, 580%, 585%, 590%, 595%, 600%, 605%, 610%, 615%, 620%, 625%, 630%, 635%, 640%, 645%, 650%, 655%, 660%, 665%, 670%, 675%, 680%, 685%, 690%, 695%, 700%, 705%, 710%, 715%, 720%, 725%, 730%, 735%, 740%, 745%, 750%, 755%, 760%, 765%, 770%, 775%, 780%, 785%, 790%, 795%, 800%, 805%, 810%, 815%, 820%, 825%, 830%, 835%, 840%, 845%, 850%, 855%, 860%, 865%, 870%, 875%, 880%, 885%, 890%, 895%, 900%, 905%, 910%, 915%, 920%, 925%, 930%, 935%, 940%, 945%, 950%, 955%, 960%, 965%, 970%, 975%, 980%, 985%, 990%, 995%, to/or 1,000% or more.

In some embodiments, the effective amount of CLA increases spermatogenesis. In some embodiments, the effective amount of CLA increases spermatogenesis about 1% to about 1,000% or more. In some embodiments, the effective amount of CLA increases spermatogenesis about 1%, 5%, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, 200%, 205%, 210%, 215%, 220%, 225%, 230%, 235%, 240%, 245%, 250%, 255%, 260%, 265%, 270%, 275%, 280%, 285%, 290%, 295%, 300%, 305%, 310%, 315%, 320%, 325%, 330%, 335%, 340%, 345%, 350%, 355%, 360%, 365%, 370%, 375%, 380%, 385%, 390%, 395%, 400%, 405%, 410%, 415%, 420%, 425%, 430%, 435%, 440%, 445%, 450%, 455%, 460%, 465%, 470%, 475%, 480%, 485%, 490%, 495%, 500%, 505%, 510%, 515%, 520%, 525%, 530%, 535%, 540%, 545%, 550%, 555%, 560%, 565%, 570%, 575%, 580%, 585%, 590%, 595%, 600%, 605%, 610%, 615%, 620%, 625%, 630%, 635%, 640%, 645%, 650%, 655%, 660%, 665%, 670%, 675%, 680%, 685%, 690%, 695%, 700%, 705%, 710%, 715%, 720%, 725%, 730%, 735%, 740%, 745%, 750%, 755%, 760%, 765%, 770%, 775%, 780%, 785%, 790%, 795%, 800%, 805%, 810%, 815%, 820%, 825%, 830%, 835%, 840%, 845%, 850%, 855%, 860%, 865%, 870%, 875%, 880%, 885%, 890%, 895%, 900%, 905%, 910%, 915%, 920%, 925%, 930%, 935%, 940%, 945%, 950%, 955%, 960%, 965%, 970%, 975%, 980%, 985%, 990%, 995%, to/or 1,000%.

In some embodiments, the effective amount of the CLA decreases testicular degeneration. In some embodiments, the effective amount of the CLA decreases testicular degeneration by about 1%-1,000% or more. In some embodiments, the effective amount of the CLA decreases testicular degeneration by about 1%, 5%, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, 200%, 205%, 210%, 215%, 220%, 225%, 230%, 235%, 240%, 245%, 250%, 255%, 260%, 265%, 270%, 275%, 280%, 285%, 290%, 295%, 300%, 305%, 310%, 315%, 320%, 325%, 330%, 335%, 340%, 345%, 350%, 355%, 360%, 365%, 370%, 375%, 380%, 385%, 390%, 395%, 400%, 405%, 410%, 415%, 420%, 425%, 430%, 435%, 440%, 445%, 450%, 455%, 460%, 465%, 470%, 475%, 480%, 485%, 490%, 495%, 500%, 505%, 510%, 515%, 520%, 525%, 530%, 535%, 540%, 545%, 550%, 555%, 560%, 565%, 570%, 575%, 580%, 585%, 590%, 595%, 600%, 605%, 610%, 615%, 620%, 625%, 630%, 635%, 640%, 645%, 650%, 655%, 660%, 665%, 670%, 675%, 680%, 685%, 690%, 695%, 700%, 705%, 710%, 715%, 720%, 725%, 730%, 735%, 740%, 745%, 750%, 755%, 760%, 765%, 770%, 775%, 780%, 785%, 790%, 795%, 800%, 805%, 810%, 815%, 820%, 825%, 830%, 835%, 840%, 845%, 850%, 855%, 860%, 865%, 870%, 875%, 880%, 885%, 890%, 895%, 900%, 905%, 910%, 915%, 920%, 925%, 930%, 935%, 940%, 945%, 950%, 955%, 960%, 965%, 970%, 975%, 980%, 985%, 990%, 995%, to/or 1,000%.

In certain example embodiments, the effective amount of CLA modulates (increases or decreases) expression, optionally the hypothalamic expression, of one or more genes. the In some embodiments, the effective amount of CLA modulates (increases or decreases) hypothalamic expression of one or more genes. In certain example embodiments, the effective amount of CLA modulates (increases or decreases) hypothalamic expression of one or more genes selected from: Ipw, Gria4, Ndm, Gm32061, Zswim5, Mael, Mfge8, Rertreg2, Mstol, Tecr, Zfp316, Spryd3, Mrps26, Hlf10, Eif6, Tmem59I, Serpina3n, Tubb2b, Slc12a2, Ppargcla, Dgkk, or any combination thereof. In certain example embodiments, the effective amount of CLA modulates (increases or decreases) expression, optionally the hypothalamic expression, of one or more genes selected from any one or more of those set forth in Supplementary Data tables S1-S3 of Knott et al., 2022. Nutrients. 14:860, which are incorporated by reference as if expressed in their entireties herein. In certain example embodiments, the effective amount of CLA modulates a biologic program or pathway, such as any of those set forth in any one of Tables 2-4.

In some embodiments, the effective amount of the CLA decreases anxiety or a symptom thereof. In some embodiments, the effective amount of the CLA decreases anxiety or a symptom thereof by about 1%-1,000% or more. In some embodiments, the effective amount of the CLA decreases anxiety or a symptom thereof by about 1%, 5%, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, 200%, 205%, 210%, 215%, 220%, 225%, 230%, 235%, 240%, 245%, 250%, 255%, 260%, 265%, 270%, 275%, 280%, 285%, 290%, 295%, 300%, 305%, 310%, 315%, 320%, 325%, 330%, 335%, 340%, 345%, 350%, 355%, 360%, 365%, 370%, 375%, 380%, 385%, 390%, 395%, 400%, 405%, 410%, 415%, 420%, 425%, 430%, 435%, 440%, 445%, 450%, 455%, 460%, 465%, 470%, 475%, 480%, 485%, 490%, 495%, 500%, 505%, 510%, 515%, 520%, 525%, 530%, 535%, 540%, 545%, 550%, 555%, 560%, 565%, 570%, 575%, 580%, 585%, 590%, 595%, 600%, 605%, 610%, 615%, 620%, 625%, 630%, 635%, 640%, 645%, 650%, 655%, 660%, 665%, 670%, 675%, 680%, 685%, 690%, 695%, 700%, 705%, 710%, 715%, 720%, 725%, 730%, 735%, 740%, 745%, 750%, 755%, 760%, 765%, 770%, 775%, 780%, 785%, 790%, 795%, 800%, 805%, 810%, 815%, 820%, 825%, 830%, 835%, 840%, 845%, 850%, 855%, 860%, 865%, 870%, 875%, 880%, 885%, 890%, 895%, 900%, 905%, 910%, 915%, 920%, 925%, 930%, 935%, 940%, 945%, 950%, 955%, 960%, 965%, 970%, 975%, 980%, 985%, 990%, 995%, to/or 1,000%.

In some embodiments, the effective amount of the CLA, modulates the α-diversity of the gut microbiome. As used in this context herein, α-diversity describes the structure of a microbial community in relation to the number of taxonomic groups and their respective abundance.

The CLA can be formulated for any suitable route of administration including, but not limited to, oral (including buccal or sublingual), intranasal, topical, parenteral, subcutaneous, intramuscular, intravenous, internasal, and intradermal. Such formulations can be prepared by any method known in the art. The CLA or formulation thereof can be administered by any suitable routes. Exemplary suitable administration routes are described in greater detail elsewhere herein, such as in relation to the CLA compositions previously described. In some embodiments, administration is oral.

In certain example embodiments, the dietary supplement, feed, or foodstuff or beverage formulation is a liquid, solid, semi-solid, or an emulsion. In some embodiments, the dietary supplement, feed, or foodstuff or beverage formulation is a powder or contains a powder. In some embodiments, the powder is formulated to be reconstituted or diluted in a liquid or emulsion. In some embodiments, the beverage formulation is a syrup, drink or shake, or other liquid formulated for oral consumption. Other exemplary dosage forms, formulations and/or the like are described in greater detail elsewhere herein.

In certain example embodiments, the subject in need thereof is a mammal, optionally a human.

Administration can be one or more times hourly, daily, monthly, or yearly (e.g., 1, 2, 3, 4, 5, 6, 7, 8, 9, 10, 11, 12, 13, 14, 15, 16, 17, 18, 19, 20, or more times hourly, daily, monthly, or yearly). In some embodiments, administration can be 1-3 times daily. In some embodiments, the CLA or formulations thereof or dosage forms thereof described herein can be administered continuously over a period of time ranging from minutes to hours to days. In some embodiments, administration can continue as long as desired. In some cases, this may be days, weeks, months, or for life or for as long as the disease or symptoms thereof persist.

Further embodiments are illustrated in the following Examples which are given for illustrative purposes only and are not intended to limit the scope of the invention.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure. The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

### Example 1—Dietary CLA Effects in Snord116^{m+/p-} and Snord116^{m-/p-} Mouse Models of Prader-Willi Syndrome

### 1. Introduction

Prader-Willi Syndrome (PWS) is a genetic condition that occurs in up to 1 in 10,000 live births [1]. Individuals with PWS show initial developmental delay, significant hypotonia/muscle weakness, and typically demonstrate some level of intellectual disability, and obesity later in childhood or adolescence. The most common cause of the syndrome is a de novo deletion of the paternal 15q chromosome, as the maternal allele is imprinted and not expressed. Uniparental (maternal) disomy and an imprinting locus mutation can also be causative. A minimal deletion of chromosome 15q that causes PWS includes just two expressed regions: the SNORD116 cluster (a group of 28 or more small nucleolar RNAs “snoRNAs”), and the IPW gene which also encodes a non-coding RNA of little known function [2]. For PWS patients, there is, as yet, no cure, and few treatment options.

In late 2016, Burnett and colleagues showed that NHLH2 and PCSK1 (PC1/3) mRNAs and proteins were downregulated in PWS-derived induced pluripotent stem cell neurons (iPSC), and in the PWS mouse model, the Snord116m+/p− mouse [3]. Likewise, PWS patients have a 1.5-fold reduction in NHLH2 expression in lymphoblastoid cells [4]. It was recently demonstrated that Nhlh2 mRNA is indeed upregulated post-transcriptionally by Snord116 snoRNA [5].

Interestingly, mice with a deletion of Nhlh2 (N2KO mice), which were developed in 1997 [6], share multiple phenotypes with the PWS Snord116m+/p− mouse model, which has a deletion of Snord116 only on the paternally inherited allele [7]. However, the Snord116m+/p− mouse (PWS mouse) fails to develop overt obesity on regular mouse chow, but they may develop hyperphagia by three months of age [7]. A mouse model with hypothalamic-only adult deletion of Snord116 does develop both hyperphagia and later-onset obesity [8], and there are ˜25 different PWS mouse models containing genetic manipulation with the mouse chromosome 7 regions homologous to human 15q—each with varying similarity to the human condition [9]. The PWS Snord116m+/p− mouse model was chosen as a model for this study as it is most similar genetically to the minimal deletion in humans that results in PWS phenotypes, and a mouse with hypothalamic adult Snord116 conditional deletion develops increased adiposity and hyperphagia [8]. Additionally, the PWS Snord116m+/p− mouse model has most recently been used in a study to test the use of growth hormone therapy as a treatment option for PWS patients [10]. Finally, Qi and colleagues previously published on the body weight and food intake characteristics of the PWS KO mouse (Snord116m−/p−) [11], which was included in this study to analyze complete absence of the Snord116 alleles. These results could mimic an individual carrying one deleted Snord116 allele, along with an inactivating variant in Snord116 or another PWS locus gene. One such patient with a homozygous variant in SNURF-SNRPN gene in the PWS locus has been described [12].

Previously, it was demonstrated that conjugated linoleic acid (CLA) treatment of N2KO mice on a 20% fat diet led to weight loss, decreased body fat, increased metabolism, muscle mitochondrial biogenesis, and increased exercise performance [13-16]. At the time of the initial paper, these outcomes represented the first time that genetic obesity was “cured” with CLA. CLA is currently considered to be safe (GRAS, “generally regarded as safe”) by the FDA and is available over the counter. However, results from human studies have been variable, which could be due to differences in dosage, human genetics, and small study sample sizes [17].

Aside from growth hormone [18], there are few treatment options for individuals with PWS. Given the overlap in phenotypes between the N2KO, Snord116m+/p− mouse model, and PWS in humans, and the finding that Nhlh2 is downstream of Snord116, it was sought to determine if deletion of Snord116 in the Snord116m+/p− mouse model affected the response to dietary CLA. With later-onset obesity and inactivity being clinically relevant features of PWS, CLA could prove to be a potential cost-effective treatment for patients.

### 2. Materials and Methods

### 2.1 Mouse Housing and Husbandry

All animal protocols were approved by the Institutional Animal Care and Use Commit-tee at Virginia Tech. Mice with the Snord116 paternal deletion (B6[Cg]-Snord116tm1.1Uta/J Stock No: 008149|1-loxp (KO), Snord116del) were obtained from Jackson Laboratories and maintained on a C57Bl/6 background. Genotyping and breeding were performed as reported [7]. WT mice for the study were siblings of either the PWS (Snord116m+/p−) or PWS-KO (Snord116m−/p−) mice. PWS mice were generated by crossing WT females and male mice who were Snord116m−/p+. Breeder males (Snord116m−/p+) were generated in separate crosses to inherited their deleted allele from a maternal chromosome so that they were considered phenotypically normal in regards to Snord116 expression. PWS-KO (Snord116m−/p−) mice were generated by using non-sibling male and female mice of the genotype Snord116m−/p+ who had inherited their deleted allele maternally. In this type of mating, 50% of the offspring would be heterozygous (and never used for mating or the study, as the origin of their deleted allele could not be determined), and 25% each would have WT alleles or would be PWS-KO. Mice were housed at room temperature at about 22° C. with 12 h light/dark cycles at 7 a.m. and 7 p.m. and ad-libitum access to food (4.5% crude fat) and water. Mice were weaned at 3 weeks old, genotyped using an ear snip, thanized by CO2 asphyxiation between 12 p.m. and 2 p.m. ear tagged, and housed with littermates of the same sex. For tissue dissection, mice were euthanized by CO2 asphyxiation between 12 p.m. and 2 p.m.

TABLE 1
Experimental Treatment Groups

| | Treatment 20% | Treatment 20% |
|---|---|---|
| Genotype | Fat Diet | Fat Diet with CLA |
| WT | 8 | 8 |
| PWS (Snord116^{m+/p-}) | 8 | 8 |
| PWS KO (Snord116^{m-/p-}) | 7 | 8 |

### 2.3 Weekly Measurements

Body weight, fat, and lean mass, rectal temperature, and food intake were measured weekly. For body weight, fat, and lean mass measurements, mice were weighed on Mondays using a standard scale, and then placed in a Bruker (Billrica, MA, USA) LF90 NMR machine using the mouse holder. Measurements were recorded in grams for fat and lean mass.

### 2.4 Study Pre- and Post-Measurements

Wheel running, elevated plus maze anxiety-like behavior testing, and glucose tolerance measurements were conducted in both the pre- and post-weeks of the study. Each are described separately below.

### 2.4.1 Wheel Running

For the spontaneous/motivated wheel running analysis, mice were placed in wire-bottom cages equipped with computer-monitored running wheels (Mini-mitter, Sunriver, OR, USA) for a total of 4 days, and then returned to their home cages. The first two days were not recorded as these were considered acclimation days, and the last 48 h were used for data collection. Mice had ad lib access to food and water during this time. For the pre-week, regular mouse chow was provided. For the post-week measurements, mice were supplied with their study diet during testing.

### 2.4.2 Elevated Plus

A plus-shaped elevated plus apparatus with two open and two closed arms was constructed using measurements obtained from a commercial apparatus. This elevated plus maze was used for anxiety-like behavior analysis [19]. The maze was cleaned with 70% ethanol before and after the procedure for each mouse and allowed to fully dry between mice. Mice were placed in the center/cross-arms and allowed to freely explore the maze for 5 min while the duration and frequency of entries into open and closed arms was recorded. Animals that enter the open arms more frequently are considered to display less overall anxiety-like behavior than those that stay in the closed arms [19].

### 2.4.3 Glucose Tolerance Tests

Mice were food-deprived for 12-15 h during the dark cycle in a cage devoid of bedding material. For the fasting measurement, the tail was snipped (1-2 mm), and blood was collected directly on Care Touch glucose test strips and directly measured using a Care Touch blood glucose monitor (Future Diagnostics USA, Brooklyn, NY, USA). Glucose (pharmaceutical grade dextrose, 2 g/kg in PBS, sterile) was injected intraperitoneally (IP) and tail blood samples were obtained at 15, 30, 60, 90, and 120 min following the injection. Area under the curve was calculated using the formula (((([fasting glucose×7.5]+[15 min glucose×15)+[30 min glucose×22.5])+[60 min glucose×30])+[90 min glucose×30])+[120 min glucose×15]).

### 2.5 End-of-Study Measurements

Rotarod balance measurements, metabolic measures, mouse functional muscle testing, euthanasia with blood collection, tissue collection, and histology were all performed only as post-measurements at the end of the study during Weeks 13-14. Mice continued on their study diet until euthanasia.

### 2.5.1 Rotarod Analysis

For rotarod analysis, animals were tested using a Columbus Instruments (Columbus, OH, USA) Economex Rota-Rod apparatus. All mice in the study were given four trials each day during the day (between 11 a.m. and 1 p.m.) for four consecutive days from 0 rpm to a maximum speed of 20 rpm, with an acceleration slope of 2.65%. Animals were tested for a maximum of 5 min of running per test, or until the animals fell off the device. The Economex Rota-Rod apparatus is equipped with a pressure-sensitive landing area so that the time spent on the rotating rod is automatically recorded when the animal falls.

The procedure has been previously described [20]. Animals were given between 10 and 15 min of rest in between trials. Note that there are four days of testing, with each day consisting of four trials. The initial trials on Day 1 use a lane where Applicant placed non-slip bathroom tape to provide a stable environment for the animal to learn the procedure. The non-taped lanes were used for Days 2-4. Days 2-3 serve as acclimation days. Day 4 data were used for analysis.

2.5.2 Indirect calorimetry and Home-Cage Activity

Indirect calorimetry and locomotor activity measurements were performed using a Labmaster Mouse calorimetry and Locomotor system (TSE Systems, Bad Homburg, Germany) in the Metabolism Core Virginia Tech. VO2 consumption and VCO2 production in individual mice were measured using metabolic chambers. Air going into the TSE system was at 20.9% oxygen, 0.05% CO2, and the airflow rate was 0.4 L/min (Airgas, Christiansburg, VA, USA). Data were collected every 15 min. Body composition was measured, as described above, prior to assessment of the animals using the TSE system. A photobeam-based ac-tivity monitoring system detected and recorded ambulatory movements. The results were used to calculate the respiratory exchange ratio (RER) and total energy expenditure/gram lean mass. Energy expenditure (KJ/h) was calculated using the formula VO2×(3.815+(1.232×RER))×4.1868 and normalized to the lean mass determined by NMR. All parameters were measured continuously and simultaneously for 48 h after approximately 20 h of adaptation for single-housed mice. The average values for the last 24-48 h were used for analysis. To calculate hourly activity, the average of two-hour bins of activity from the last 24 h period was plotted. Activity level was plotted separately as time of rest (activity level=0) beam breaks per 15 min bin) or activity (activity level equal to or greater than 100 beam breaks per 15 min bin) and summed for the 24 h period. Due to equipment malfunc-tion occurring during the study, calorimetry was performed on only the following numbers for each genotype and treatment: N=7 WT control, N=5 WT CLA, N=6 PWS control, N=6 PWS CLA, N=4 PWS-KO control, N=4 PWS-KO CLA. These reduced n values may have contributed to the non-significant finding for the indirect calorimetry assessment.

### 2.5.3 In Vivo Muscle Function Testing

The procedures for in vivo muscle testing were recently published [22]. Briefly, at the end of the study, following both running wheel and metabolism measurements, body weight was determined, and mice were anesthetized with isoflurane (VetOne Fluriso, Boise, ID, USA) and placed on the temperature-controlled platform (40° C.) of the contractile apparatus (ASI), as described. The right hindlimb was shaved, hair remover applied (Nair Hair Remover Lotion, Ewing, NJ, USA) for 30 s, cleaned with 2-inch×2-inch gauze and tap water, and swabbed with povidone-iodine (Betadine Solution Swabsticks, Stamford, CT, USA). The knee was clamped so the tibia was 90° to the femur. The foot at 90° to the tibia was secured with clear Transpore surgical tape (M3, St. Paul, MN, USA) to the foot pedal of the Aurora Scientific (ASI: Aurora, ON, Canada) dual-mode servomotor. The mouse tail was taped (M3, St. Paul, MN, USA) loosely to the platform to keep it clear of the foot pedal. In vivo plantarflexor torque-frequency and fatigue assays were determined as described in Hamm et al. [22]. Dynamic Muscle Control (DMC) software controlled the timing and frequency of the stimulations and collection of torque. Peak torque for each muscle contraction was determined using ASI Dynamic Muscle Analysis (DMA) software. Due to the body mass and fat mass reduction of the CLA groups in this study, torque was normalized separately by body mass (g) and the final measure of lean body mass (g).

### 2.6 RNA Isolation

Fresh hypothalamus tissue was lysed in TRIzol® using a rotor stator homogenizer. TRIzol samples were frozen at −20° C. in microfuge tubes until purified (2 weeks to 5 months). Thawed TRIzol samples were purified using the TRIzol+Purelink RNA minikit (ThermoFisher, Waltham, MA, #12183025), following manufacturer's instructions for the TRIzol® Plus Total Transcriptome Isolation protocol. Purified RNA was then DNAse-treated using TURBO DNA-free™ Kit (ThermoFisher #AM1907) according to manufacturer's instructions, diluted to 60 ng/μL in nuclease-free water, and stored at −80° C.

### 2.7 RNA-Seq

RNA sequencing and library preparation was performed by Virginia Tech's Genomics Sequencing Center facility at the Fralin Life Sciences Institute. Total RNA with an RNA Intensity Number ≥8.0 was converted into a strand-specific library using Illumina's TruSeq Stranded mRNA HT Sample Prep Kit (Illumina, San Diego, CA, RS-122-2103), for subse-quent cluster generation and sequencing on Illumina's NextSeq. The library was enriched by 14 cycles of PCR, validated using Agilent TapeStation, and quantitated by qPCR. Individually indexed cDNA libraries were pooled and sequenced on NextSeq 75 SR. The Illumina NextSeq Control Software v2.1.0.32 with Real Time Analysis RTA v2.4.11.0 was used to provide the management and execution of the NextSeq 500 and to generate binary base call (BCL) files. The BCL files were converted to FASTQ files, adapters trimmed, and demul-tiplexed using bcl2fastq Conversion Software v2.20. FASTQ files were aligned to mouse genome GRCm38.p6 using the Geneious RNA assembler 2 Jan. 2020 from Geneious Prime with map quality 30 (99.9% confidence) and spanning intron annotations. Raw read counts were quantified to gene and/or transcript annotations with loss of strand-specificity. Differential expression analysis across experimental conditions was performed using the DESeq2 plugin in Geneious Prime [23-25]. Default settings for filtering low-expression mapped genetic features were used for the DESeq2 plugin. False discovery rate (FDR) was calculated using the Benjamini-Hochberg procedure with a threshold for significance of FDR <0.10. Additionally, raw read counts were used to quantify differential expression through edgeR and voom using the DEApp online webserver [26]. Low-expression mapped genetic features were removed with log 2CPM <1 in 2 or more samples. The results from the three differential expression methodologies, DESeq2, edgeR, and voom, were compared using R and spreadsheet software such as Microsoft Excel. Differentially expressed genes were analyzed for gene-ontology enrichment with MouseMine [27].

### 2.8 Reverse-Transcriptase Quantitative PCR

For RT-QPCR, a Power SYBR® Green RNA-to-CT™ 1-Step Kit (ThermoFisher #4389986) was used according to manufacturer's instructions. Reactions of 10 μL were performed using 150 nM final primer concentration. Primers were assessed for efficiency using a dilution series and fell within 90-110% efficiency. A 90 ng measure of RNA was used per 10 μL reaction. Two to three technical replicates were performed. Control reactions for each sample (minus reverse-transcriptase and minus template controls) were used for quality control. On the ViiA 7 Real-Time PCR System (ThermoFisher), 384-well plates were run according to RT-QPCR mix instructions, and thermocycling conditions were not modified from suggested protocol (one-step annealing/extension at 60° C.). Quality-control measures including melt-curve analysis, technical replicate analysis, etc., were analyzed by thermocycler software and by operator; any major errors were excluded from analysis when deemed appropriate by the quality-control software, and/or new samples and plates were run. Candidate reference genes for ddCT analysis were analyzed and mouse beta-actin was chosen as the reference gene control for all experiments.

### 2.9 Microbiome Analysis

Cecal samples were flash-frozen in liquid nitrogen and stored at −80° C. DNA was isolated from cecal content using a DNeasy PowerSoil Pro Kit (Qiagen #47014) and the TissueLyser II (Qiagen #85300). Fifty nanograms of genomic DNA was utilized for amplifi-cation of the V4 variable region of the 16S rRNA gene using 515F/806R primers. Forward and reverse primers were barcoded to accommodate multiplexing up to 384 samples per run as described by Kozich and colleagues [28]. Paired-end sequencing (2×250 bp) of pooled amplicons was carried out using an Illumina Miseq platform with ˜30% PhiX DNA.

Demultiplexing, adapter trimming, and generating the fastq files were performed au-tomatically using the Miseq Reporter on the instrument computer. Bioinformatics analysis was then conducted using the QIIME 2 platform [29]. Denoising was performed with an initial quality filtering followed by the Deblur algorithm [30,31]. Representative amplicon sequence variants (ASVs) were used to generate a phylogenetic tree with FastTree [32], and taxonomy was assigned using a Naive Bayes classifier trained on the Greengenes 13_8 reference [33]. To evaluate diversity metrics, samples were rarefied to an even depth of 4397 quality-filtered reads. Beta diversity was assessed using the weighted Unifrac distances and visualized using the principal coordinate analysis (PCoA) plot.

### 2.10 Histology

Tissues were isolated immediately following euthanasia by CO2 asphyxiation and placed into 4% paraformaldehyde, overnight, with rocking at 4° C. The tissues were then rinsed in 70% ethanol and stored in 70% ethanol at 4° C. until processing. The Virginia Tech Veterinary Teaching Hospital at the Virginia-Maryland College of Veterinary Medicine processed the tissues for histology and stained with hematoxylin-eosin stain for microscopy. Representative samples were visualized using a 40× ocular on a Nikon Eclipse 50i micro-scope and captured using an Olympus Q-color3 camera.

### 2.11 Statistical Analysis

All values were expressed as mean+SEM unless indicated otherwise. Comparison of means between groups and calculation of p-values were made using JMP Pro15 software (Cary, NC, USA), with Tukey's post hoc analysis for multiple comparisons when overall p-values in the comparison were significant. For the responses of weight, fat, lean mass, temperature, and food intake shown in figures, a fitted least squares regression with effects of genotype, treatment, and genotype×treatment was analyzed for Week 12 data only. Responses of these dependent variables were also analyzed across the 12-week time period, adding in effects of week, week×treatment, and week×genotype. These results are shown in supplemental data as individual data tables. The responses that had pre-post measures (wheel running, elevated plus, fasting glucose, glucose tolerance) were analyzed separately for the pre and post time periods (no effect of time). Animals were grouped together for the pre time period, regardless of their assigned data, as all animals were on standard mouse chow at that time. A fitted least squares regression with effects of genotype, treatment, and genotype×treatment was analyzed for these data, and Tukey post hoc analysis performed when effects were significant. Rotarod data were collected only during the post-period and analyzed with a response of time and effects of genotype, treatment, and genotype×treatment. For area-under-the-curve calculations, the formula function in JMP was used to calculate the area for individual animals pre and post, and then fit least squares regression model used for effects of genotype, treatment, and genotype×treatment.

All RT-QPCR data were analyzed using Microsoft Excel 16 for Microsoft 365, IBM SPSS Statistics 26 for Windows, and GraphPad Prism 9.0.0. The numbers of samples in statistical tests are described in respective figures. The 2ddCT method of relative quantification was used. Statistical significance tests were performed on respective ddCT values, from which Relative Quantification values were derived. A two-way ANOVA with Bonferroni correction was used for relative expression of RNA normalized to WT or control conditions.

In vivo plantar-flexor torque-frequency and fatigue curves were analyzed using Graph-Pad Prism 9.2.0. A two-way ANOVA was performed with multiple comparisons across groups. For torque-frequency figures, a nonlinear model was fit using the sigmoidal curve with variable slope.

Statistical analyses for microbiome data were performed in R v4.0.5. Differences in beta diversity were evaluated using permutational multivariate analysis of variance (PERMANOVA) with 999 permutations and including the weighted Unifrac distance matrix. The Kruskal-Wallis test was used to evaluate alpha metrics and taxonomic differences and the Dunn's test was used to evaluate pairwise multiple comparisons, with p-values corrected for multiple testing using the false discovery rate (FDR) approach [35].

### 3. Results

### 3.1 Body Weight and Fat Mass are Reduced in PWS and PWS-KO Mice

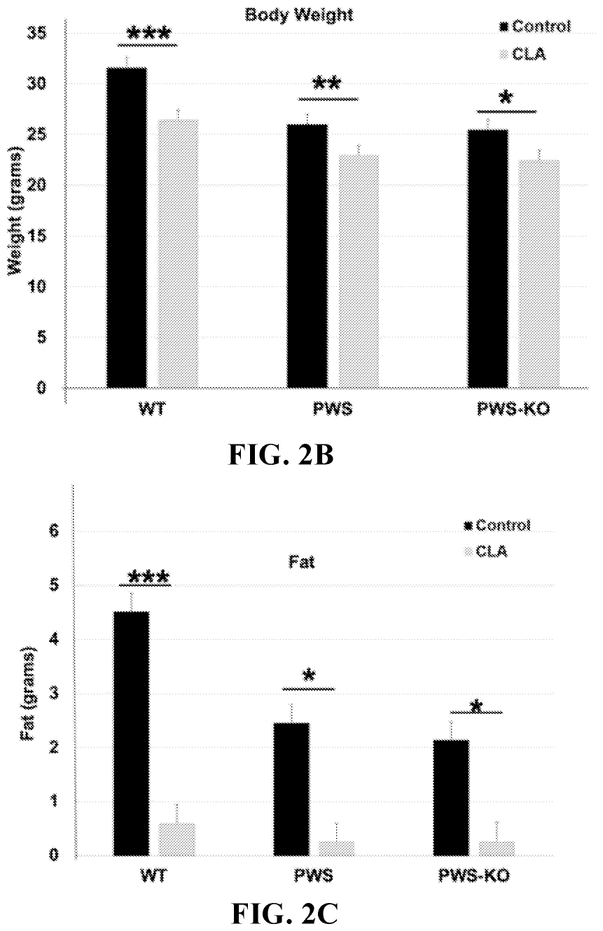

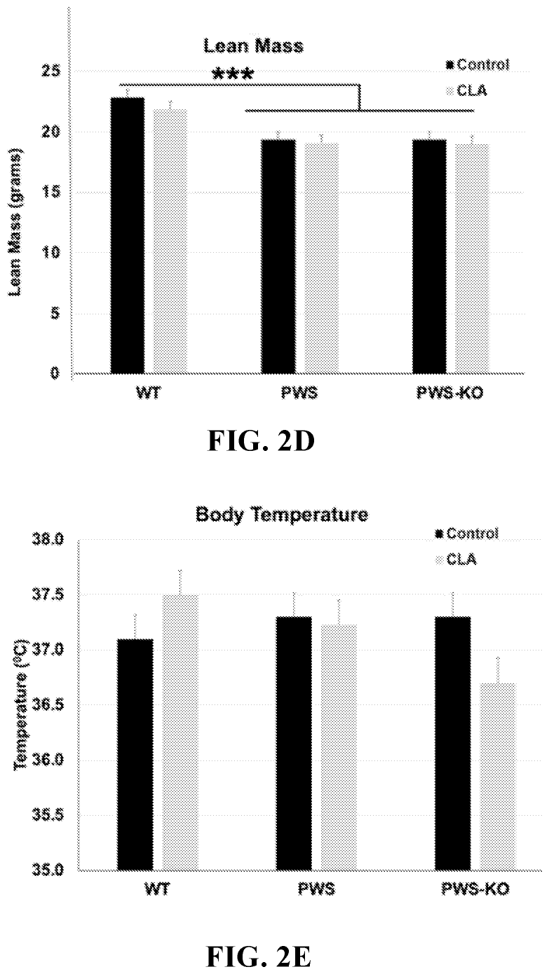

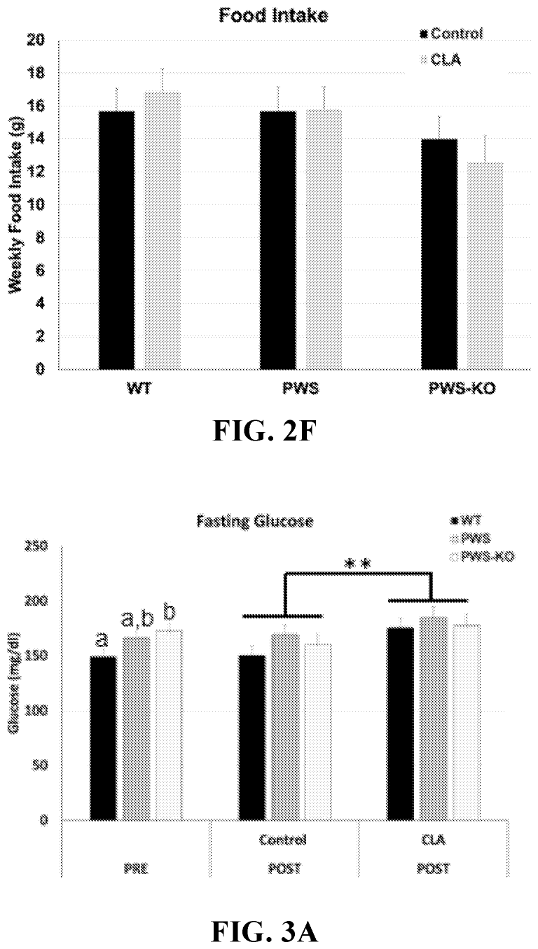

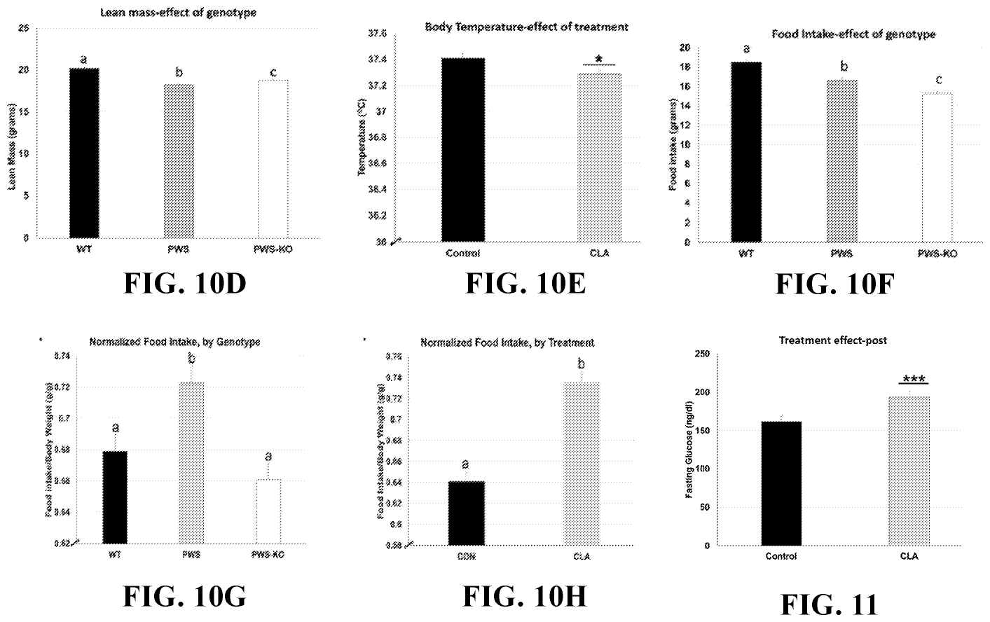

The 20% fat diet only results in a slight body weight and fat increase over a normal chow diet, and the highest weight gain occurs in the WT animals on control diet (FIG. 2A-2B). Over the 12 weeks of the study, the effects of genotype (F=234.19, p<0.001), treatment (F=179.63, p<0.001), and week (F=8.75, p<0.001) were all highly significant. Additionally, the cross effect of genotype×treatment was significant (F=4.49, p=0.0117), with post hoc analysis indicating significant differences between WT on control diet and all other genotypes and treatments (FIG. 2A). In the effect summary for body weight, response of genotype, treatment, and week were highly significant (p<0.001). Treatment by week response was significant (p=0.044), as was genotype by treatment (p=0.016). Analysis of the end of study (Week 12) data for body weight indicates that there was a significant effect of genotype (F=17.39, p<0.001) and treatment (F=23.4, p<0.001). Post hoc analysis of genotype shows significance (FIG. 2B). Although PWS and PWS-KO mice on the HFD weighed significantly less than WT mice (FIG. 10A), CLA diet still caused significant reductions in body weight overall for the three genotypes (no genotype×treatment effect, but a significant effect of treatment, FIG. 10B). Post hoc analysis for body weight (FIG. 2A) indicated that there were significant body weight reductions for each genotype on the CLA diet, as compared to control diets (F=3.70, p=0.025).

For body fat measures, over the 12 weeks of the study, the effects of genotype (F=109.77, p<0.001), treatment (F=619.05, p<0.001) (FIG. 10C), week (F=2.15, p=0.0132), and genotype by week (F=32.62, p<0.0001) were all highly significant, while the interaction of genotype by week was not (p=0.8962), indicating that genotypes all responded to the treatment similarly. In analyzing body fat at the end of the study, WT mice on the control diet had ˜4 g more body fat than WT mice on the CLA diet. Comparing fat levels to differences in body weight indicate that increased weight in control-diet groups was nearly all due to increased body fat (FIG. 2B-2C). Post hoc analysis of CLA treatment effects overall (all genotypes) on body weight indicates there was a 3.6-fold reduction in body fat levels with treatment, compared to control HFD alone (FIG. 10C).

Lean mass (lean body mass) measures muscle and organ weight, which consists of muscle, organs, bones, and fluids, constituting most of an animals' body weight. The overall effect of genotype on lean mass in the whole model was highly significant (F=52.95, p<0.001), without a treatment effect (F=0.003, p=0.96), but with both an effect of week (F=11.30, p=<0.001,), and an interaction of genotype×treatment (F=4.81, p=0.0085,). No other interactions were significant. Post hoc analysis of the genotype×treatment interaction through the entire study indicated that CLA-treated PWS-KO and PWS mice had lower overall lean mass when compared to CLA-treated WT mice (FIG. 10D). Likewise, WT control mice (19.73 g) had increased lean mass only when compared to PWS control mice (18.47 g), and not to PWS-KO controls (19.0 g). Analysis of only Week 12 lean mass levels indicates a significant effect of genotype (F=15.28, p<0.001), with Tukey post hoc analysis showing WT lean mass (22.31 g) significantly higher than both PWS (19.36 g) and PWS-KO (19.16 g) lean mass (FIG. 2C).

Body temperature readings were measured weekly over the entire study. Overall, the effect of genotype was not significant (F=1.22, p=0.29), and nor was treatment (F=2.6, p=0.11), but the effect of week was significant (F=1.8, p=0.038). However, there were no cross-interaction effects. Post hoc analysis using Student's t to examine the overall effect of week, going from pre- to post-week data, showed that there was a significant reduction in overall body temperature from Week 1 to Week 12 (Week 1, 37.54° C.; Week 12, 37.20° C., p=0.03). However, by Week 12, body temperature showed no significant differences for genotype by treatment (F=2.04, p=0.14) (FIG. 2D), while temperature over the whole study showed a significant effect of treatment (FIG. 10E).

Over the entire study, CLA diet showed a significant effect of treatment on food intake for genotype (F=41.9, p<0.0001), treatment (F=5.84, p=0.016), and week (F=9.97, p<0.0001). Post hoc analysis of genotype indicated that WT mice ate approximately 2-3 g more food overall than PWS and PWS-KO mice (FIG. 10F), but this trend is changed when food intake is normalized to body weight. Then, PWS mice have a significantly higher overall food intake/gram body weight (FIG. 10G). CLA diet overall significantly increased food intake in all genotypes (16.48 g, control diet versus 17.18 g CLA diet). This increase for CLA diet intake was true even when food intake was normalized to body weight (FIG. 10H). When analyzing food intake only at the end of the study (Week 12), there was no overall significant difference between genotypes (F=2.41, p=0.10), treatments (F=0.01, p=0.98), or the interaction of genotype_treatment (F=0.41, p=0.66).

3.2 Changes in Fasting Glucose and Glucose Tolerance with Genotype and Treatment

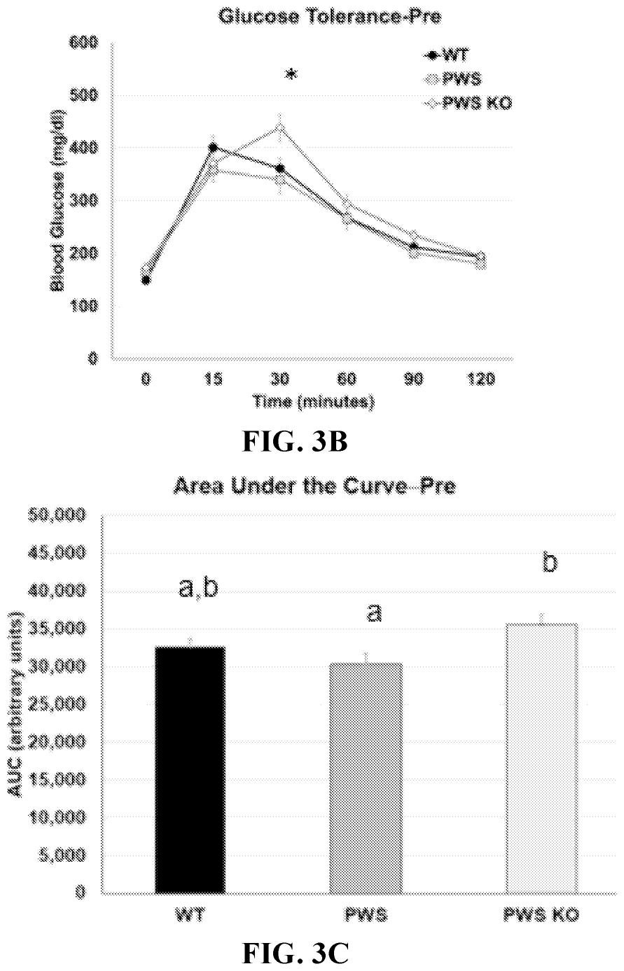

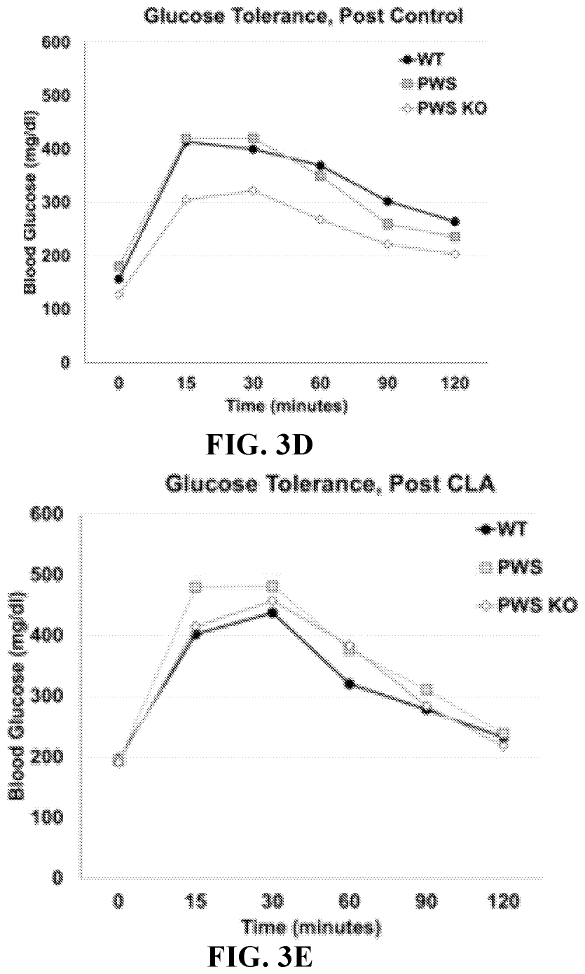

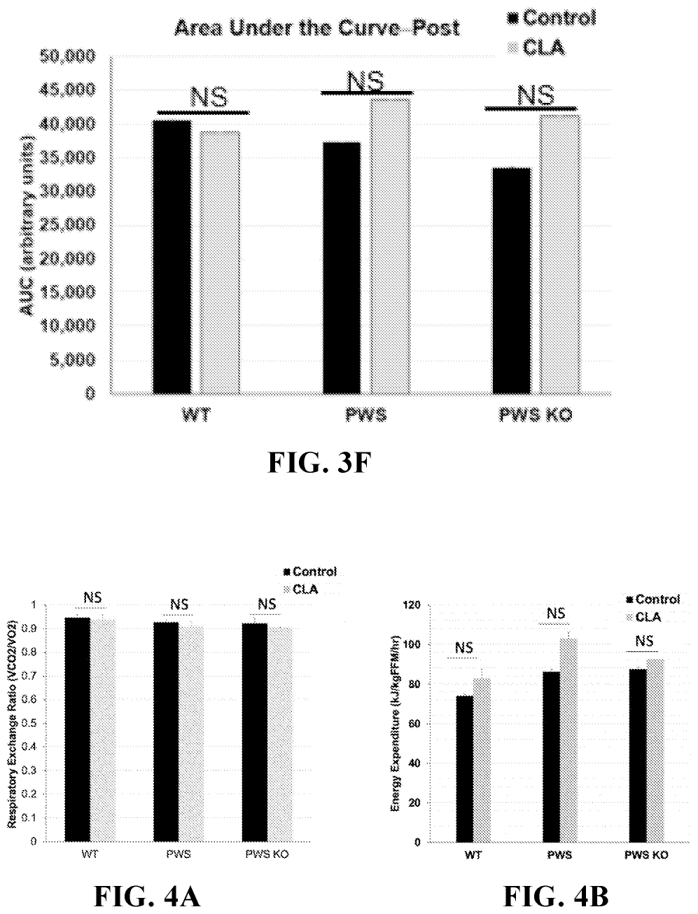

In two previous studies, glucose tolerance was improved by treatment with the 19, 11 isomers of CLA [16]. Glucose tolerance tests were performed both before diet treatment began (“Pre” Week) and at study end (Week 12). In the Pre Week, there was a significant effect of genotype on fasting glucose levels (F=4.3, p=0.0202), with post hoc test revealing a significant increase in fasting glucose for PWS-KO mice over WT, but not PWS mice (FIG. 3A). For the GTT curves, area-under-the-curve (AUC) measurements were significant for genotype (F=4.19, p=0.02), with the post hoc analysis showing significant difference between PWS and PWS-KO genotypes, but not WT mice (FIG. 3B-3C). Following diet treatment for 12 weeks, only fasting glucose levels showed a significant treatment effect (F=7.5, p=0.0092). Post hoc analysis revealed that fasting glucose for animals fed CLA diet (all genotypes) was significantly higher compared to control-diet-fed animals (FIG. 3A). However, overall AUC levels were not significant for single or cross-effects (FIG. 3D-3F).

### 3.3 No Effect of CLA Treatment on Metabolism

According to a review of CLA effects on skeletal muscle metabolism, there are mixed effects on resting metabolic rate (RMR) in humans given CLA, although mice treated with CLA show consistently higher RMRs and respiratory quotient, a measure of fat oxidation [17]. TSE measures of metabolism were performed during Week 12 of the study. At this time, home-cage activity was also measured, and body composition for all mice was measured prior to putting them in TSE chambers so that energy expenditure per fat-free mass (lean mass) could be calculated. In this study, data from several mice could not be obtained during the Post-Week period due to a malfunction in the TSE system. Analysis of the available data (N between four and seven for each group) indicated there were no significant effects of genotype, treatment, and genotype×treatment on respiratory exchange ratio (RER) (VCO2/VO2) or on KJ per fat-free mass energy expenditure levels (supplementary data tables) (FIG. 4A-4B). RER levels closer to 0.9 for all genotypes and treatments indicated higher fat oxidation, consistent with the HFD for the study (FIG. 4A). There were slight but non-significant increases for energy expenditure for all genotypes with CLA diet, and these could be contributing at some level to the lower body weight and fat in the CLA-diet animals.

3.4 CLA Modulates Spontaneous Wheel Running Levels without Effects on End-of-Study Rotarod, Home-Cage Activity, or Anxiety Measurements

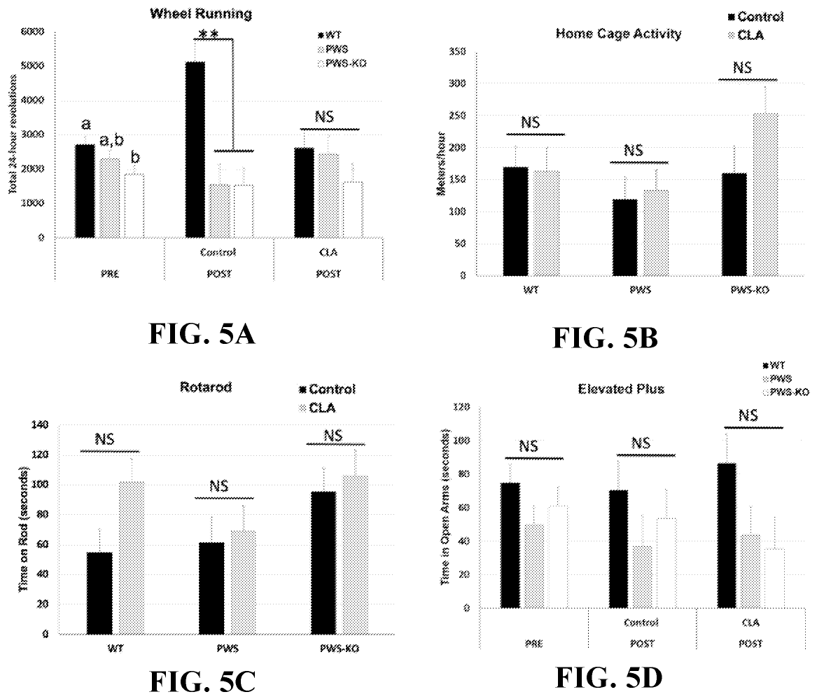

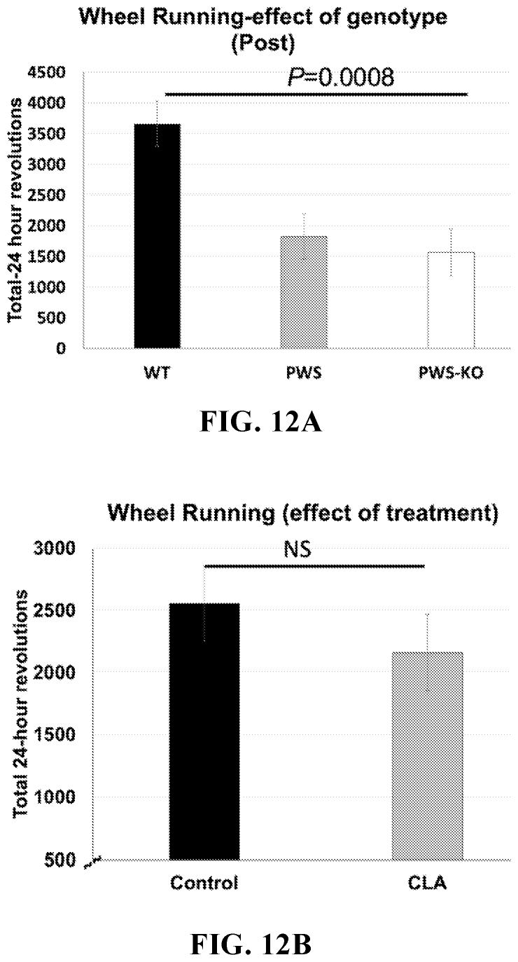

Voluntary physical activity can be increased in Nhlh2-knockout mice exposed to CLA diet [14]. Applicant sought to determine if CLA diet would also increase voluntary and home-cage activity in PWS and PWS-KO mice on HFD. In addition, mice were tested on the rotarod to confirm balance and forward-motion abilities, and with an elevated plus apparatus to analyze anxiety-like behavior. Prior to diet introduction, there was a reduction in 24 h spontaneous wheel running for PWS-KO mice compared to WT, with the p-value just above the alpha of 0.05, (F=3.18 p=0.0510). Post hoc analysis demonstrated greater wheel revolutions for WT compared to PWS-KO mice in the pre-study period (FIG. 5A). While PWS mice were not significantly different from either WT or PWS-KO mice, they did have an average reduction of 2103 revolutions per 24 h compared to WT mice (FIG. 5A). Following the CLA intervention, the effect of genotype (F=89.34, p=0.0005; FIG. 12A) and the interaction of genotype×treatment (F=4.08, p=0.0242; FIG. 5A) were significant, but not treatment alone (F=0.94, p=0.34; FIG. 12B). Post hoc analysis showed an interesting, significant increase in spontaneous running for WT mice on the control diet that was not found for PWS or PWS-KO mice. However, all three genotypes of mice on CLA diet had similar running wheel activity (FIG. 5A). When only genotype is considered, the effect of CLA on home-cage activity, as measured while mice were in the TSE metabolic chamber at the end of the study, was not significant (FIG. 5B). This could again have to do with the lower numbers of mice for each genotype and treatment group because of the TSE malfunction during the experimental time course, but is consistent with the 24 h wheel running levels with CLA treatment. As expected from the exercise findings, rotarod ability was not affected by genotype or CLA-diet treatment (FIG. 5C). Finally, previous work detected increased anxiety in PWS mice on normal chow, by using the elevated plus apparatus to detect less time spent in the open arms of the apparatus [36,37]. In this study, mice were tested both pre- and post-intervention. However, while WT mice appeared to spend more time in the open arms than either the PWS or PWS-KO mice, there were no differences based on genotype, treatment, or the interaction of genotype×treatment (FIG. 5D).

### 3.5 In Vivo Muscle Function Modulation by CLA Treatment

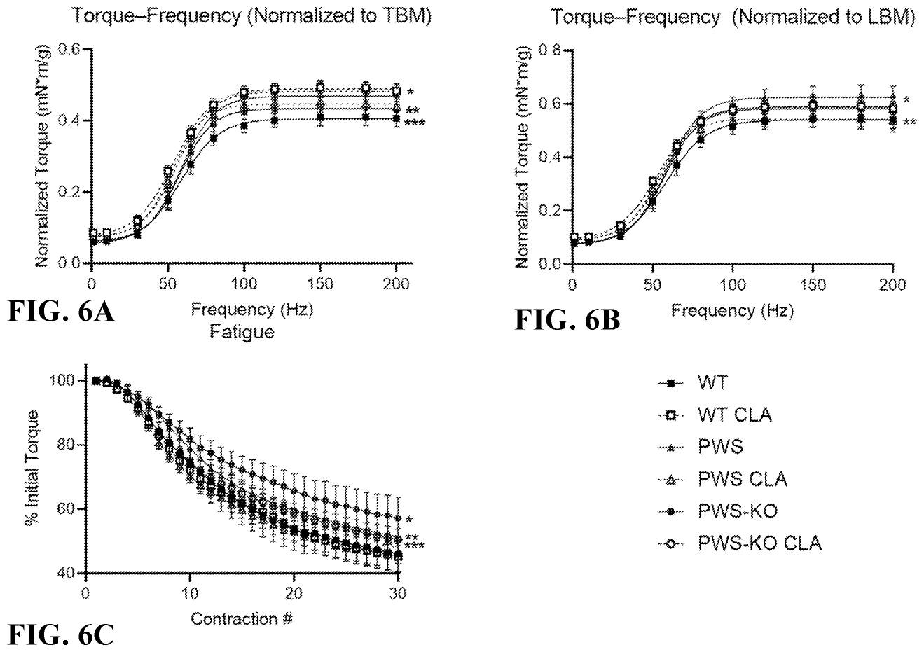

There are data suggesting that CLA can increase muscle strength in humans when combined with resistance training (e.g., [38]). At the end of the 12-week study, mice were subjected to an in vivo isometric plantar flexor torque-frequency assay to measure muscle strength in response to increasing frequency of electrical stimulation. As there were significant differences in body weight, but not lean mass of CLA groups, the data were normalized both to total body weight (FIG. 6A) and lean body mass (FIG. 6B).

Torque—frequency showed a significant effect of genotype regardless of whether it was normalized to total body weight or lean body weight (F=16.42., p<0.0001) or lean body mass (F=7.063, p<0.0001). When normalized to lean body mass or total body mass, torque was significantly reduced in WT mice on the control diet, compared to all other groups except PWS-KO (TBM p<0.0001, LBM WT CLA, PWS p<0.0001; LBM PWS-KO CLA p=0.0020). Interestingly, CLA treatment increased torque output in WT mice, regardless of normalization method (p<0.0001). PWS mice on the control diet showed a significant increase in torque over PWS mice on the CLA diet when data were normalized to lean mass (p<0.0336), but not when total body mass was used. (p=0.9993). Resistance to fatigue was greatest in the PWS-KO group (PWS p=0.0002, other groups p<0.0001). WT (PWS, PWS-KO p<0.0001, PWS-KO CLA p=0.0172) and PWS (PWS, PWS-KO p<0.0001) mice on CLA diet both showed greater fatigue than most other genotypes and treatments (FIG. 6C). CLA treatment was not detrimental to muscle function in any group, as all metrics remained greater than or equal to the WT group, which serves as a baseline.

### 3.6 CLA-Treatment Effects on Liver

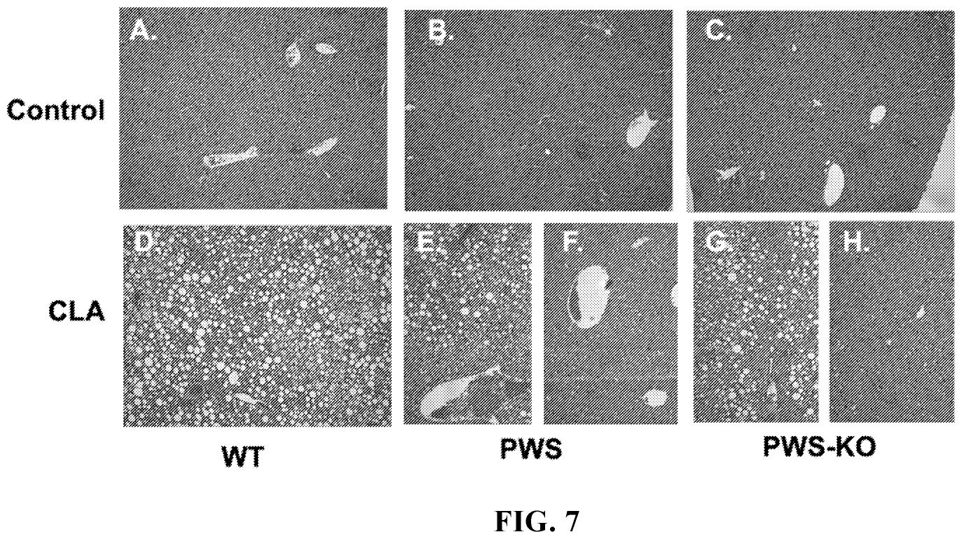

Multiple studies have shown that, in addition to fat loss, a side-effect of CLA treatment is liver steatosis, which is even more prominent with the t10,12 isomer of CLA as compared to the t9,11 isomer [39]. As Tonalin™ CLA contains a 50:50 mixture of these isomers, a finding of liver steatosis, or fatty liver, in CLA-treated mice from all genotypes was not unexpected (FIG. 7A-7H). However, as shown, while all liver sections from WT mice with CLA diet showed steatosis, only five of eight PWS mice on the CLA diet showed signs of steatosis in their livers—and two of these were mild, while four of seven PWS-KO mice on CLA diet had visible steatosis (compare FIG. 7E with FIG. 7F, and FIG. 7G with FIG. 7H).

### 3.7 RNA-Seg Analysis of Hypothalamic Gene Expression

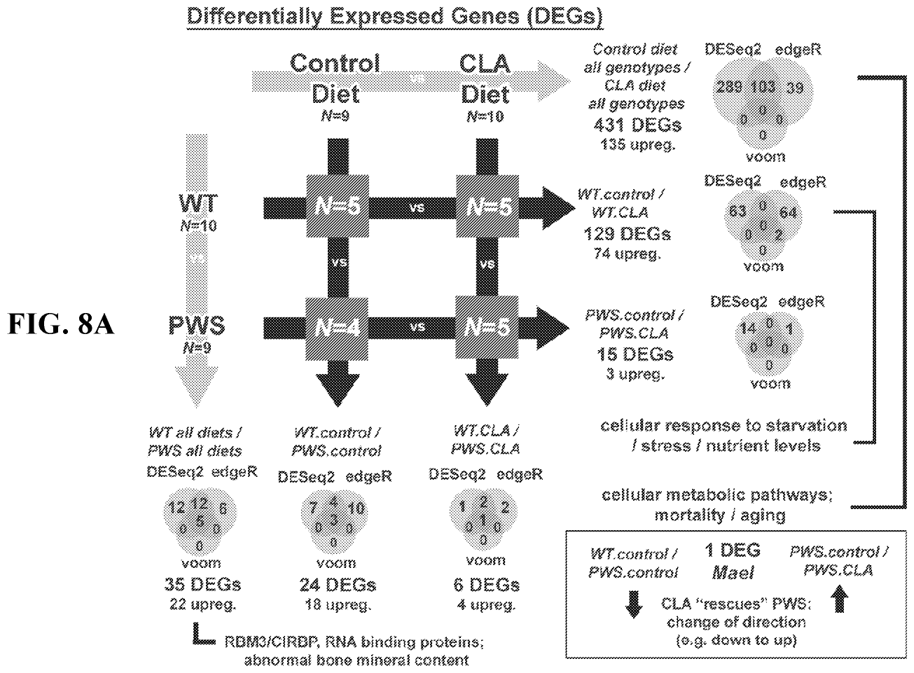

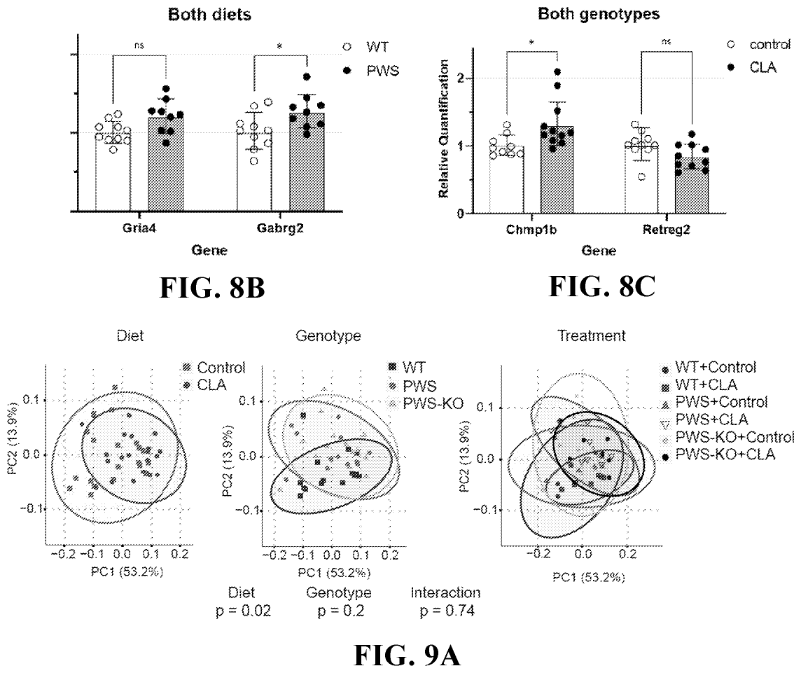

NHLH2 and SNORD116 are both highly expressed in the adult hypothalamus [40,41]. As hypothalamic dysfunction in highly implicated in PWS, this study sought to understand the effects of C:A on the PWS and WT mouse hypothalamus. RNA was isolated from dissected hypothalamic blocks to identify differentially regulated genes that may be effectors of genotype or diet. As shown in FIG. 8A, a total of 431 differentially expressed genes were found between control diet and CLA diets, while only 35 differentially expressed genes were detected when WT and PWS mice were compared. PWS-KO mice were not used in these analyses. The magnitudes of differential expression were modest for all conditions, with nearly all differentially expressed genes showing fold-changes between 0.5 and 2 (Date Tables S1-S3), although edgeR calculated higher fold-changes. Necdin (Ndn), a gene whose expression is increased by Nhlh2 [42] and whose gene locus lies within the PWS Type I (large) deletion on human chromosome 15q [9,43], was increased in PWS animals, a finding that has been reported previously [44]. Interestingly, there was no differential expression of Nhlh2 mRNA for this study, even though Applicant has shown it to be post-transcriptionally regulated by Snord116 [5]. Of all differentially expressed genes identified by RNA-seq, 135 were up-regulated by CLA diet and 22 were upregulated in the PWS genotype. Interestingly, one gene was “rescued” by CLA treatment in PWS mice, Mael, in that it is expressed in WT mice, and not in PWS except when they are treated by CLA. Additionally, the Dgkk gene was found to be overexpressed in PWS, consistent with recent in vitro findings [45]. However, these findings in Mael and Dgkk have yet to be confirmed by RT-QPCR. Gene Ontology (GO) analysis (Supplementary Tables S1-S3) revealed that CLA diet treatment resulted in significantly enriched processes related to ribosomal RNA processing, cellular metabolic processes, and aging, lethality, or survival processes (Supplementary Table S1). When only the WT response to CLA diet was examined, starvation, nutrient levels, and cellular stress were significantly enriched (Supplementary Table S2). Only two pathways, RBM3/CIRBP RNA recognition, and ab-normal bone mineral content were enriched when a genotype comparison of PWS versus WT was analyzed (Supplementary Table S3). Four differentially expressed genes were chosen using both biological function, reproducibility, and magnitude of change. These genes underwent further confirmation validation by QPCR for all diet and genotype condi-tions, with only two of the four findings replicated. As shown in FIG. 8B, the glutamate receptor, ionotropic, AMPA4 subunit (Gria4) mRNA, which was 1.23-fold up-regulated in PWS mice compared to WT mice in the original RNA-seq data, was not found to be significantly increased with QPCR (p=0.07). However, the gamma-aminobutyric acid (GABA) A receptor, subunit gamma 2 (Gabrg2) mRNA maintained its significant increase in PWS mice compared to WT mice, although the increase of 1.16-fold by QPCR is modest (FIG. 8B; p=0.02). In comparing the effect of diets on both genotypes, charged multi-vesicular body protein IB (Chmp1b) mRNA was 1.27-fold increased with CLA diet for RNA-seq, and this increase was maintained in the verification QPCR results (FIG. 8C, p=0.01). Reticulophagy regulator family member 2 (Retreg2) showed a significant 0.89-fold reduction in the RNA-seq analysis of CLA diet treatment on PWS mice, but this modest reduction was not significant in the validation QPCR results (FIG. 8C; p=0.22).

### 3.8 Cecum Microbiome Analysis in CLA and Control Diet-Treated Animals

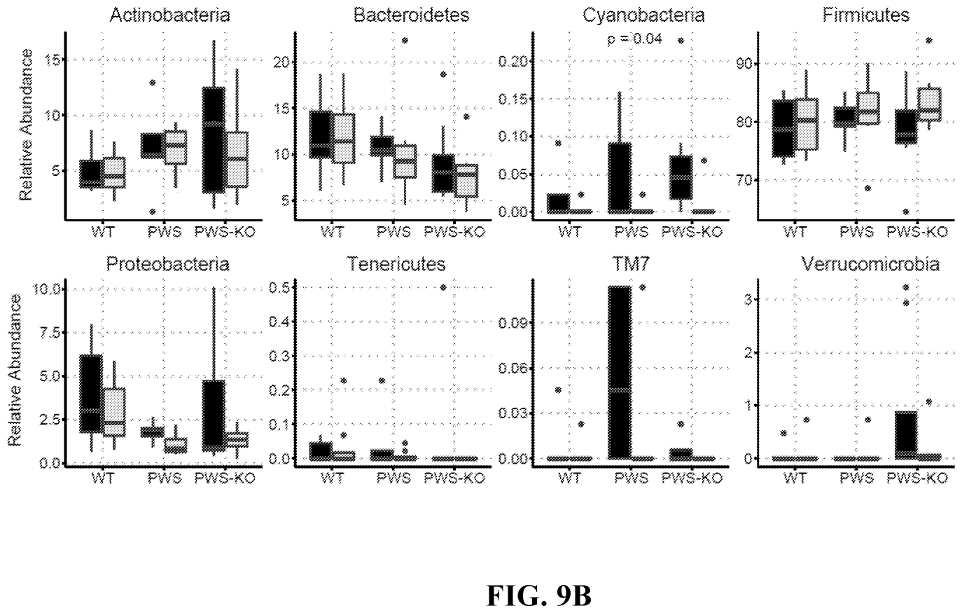

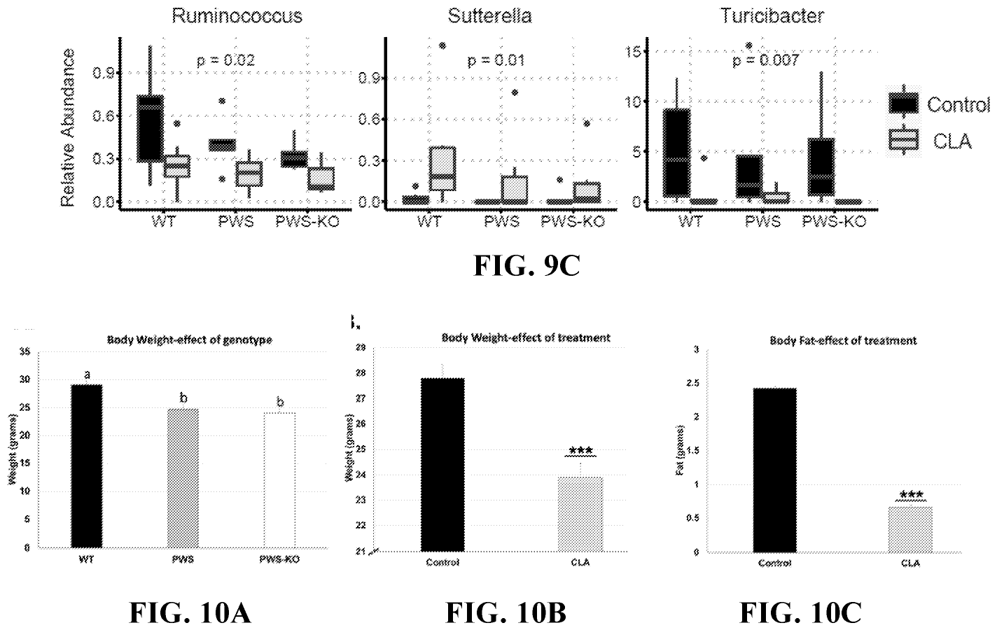

Using a mix of isomers similar to those used in this Example, Li and colleagues demon-strated a significant reduction in pro-inflammatory bacteria in CLA-fed high-fat-diet mice [46]. Other studies using human patients with multiple sclerosis have also been able to demonstrate an anti-inflammatory microbiome profile with CLA treatment [47]. Therefore, Applicant set out to determine if bacterial diversity and specific genera and species were similar or different in PWS and WT mice on CLA or control diet (FIG. 9A-9C).

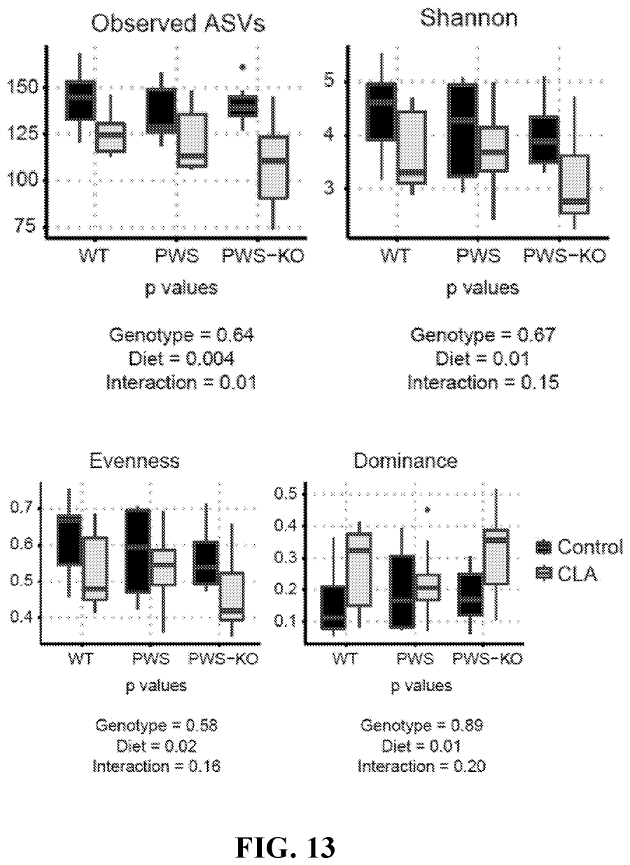

α-diversity describes the structure of a microbial community in relation to the number of taxonomic groups and their respective abundance. α-diversity metrics were similar by genotype (p≥0.58) but differed between the control and CLA treatments (p≤0.02; FIG. 13). β-diversity represents community compositional differences between samples. Here, as shown in FIG. 9A, β-diversity measures were significantly impacted by CLA diet (p>0.05), whereas genotype had no effect. At phylum level, the relative abundance of Cyanobacteria was lower in mice fed the CLA diet compared to those fed the control diet (p=0.04), whereas the relative abundance of the other phyla was similar (p>0.09) between diets. Amongst genotypes, PWS-KO mice had significantly lower relative abundance of Bacteroidetes compared to WT mice (p=0.04), but similar compared to PWS (p=0.26). Reduced abundance of Bacteroidetes phylum is generally associated with obesity [48]. For Proteobacteria, PWS mice had a lower relative abundance (p=0.05) compared to WT mice, but similar compared to PWS-KO (p=0.73) (FIG. 9B). Only three significant genera were identified in this study (FIG. 9C), with all three being impacted by diet. Ruminococcus sp. Is important for plant cell wall breakdown in the colon [49], and in this Example, was reduced with CLA treatment for all genotypes. While not much is known about Sutterella sp. and its role in gut microbiome health, the genus was significantly increased with CLA diet (H=13.8, p=0.02). Turicibacter is a genus in the Firmicutes phylum, with this phylum generally making up the largest portion of the gut microbiome [50]. CLA treatment significantly reduced Turicibacter sp. In all genotypes (H=15.7, p=0.008), although the Firmicutes phylum overall was not significantly affected (H=3.74, p=0.59).

3.9 Fasting Glucose with CLA Diet-Effect of Treatment

Glucose measurements were done at the end of the study (12 weeks). Post hoc analysis findings for fasting glucose revealed high significance (***=P<0.001) for the effect of diet (genotypes groups by diet). Data are presented as mean+/−standard error of the mean. N=8 WT control, 8 WT CLA, 8 PWS control, 7 PWS CLA, 7 PWS-KO control, 8 PWS-KO CLA, grouped by diet (FIG. 11).

### 3.10 Whole Transcript/Gene Data From RNA-Seq

Whole transcript/gene data from RNA-Seq is published as Table S1 in the Supplementary materials of Knott et al., 2022. Nutrients. 14:860 https://doi.org/10.3390/nu14040860, which is incorporated by reference as if expressed in its entirety herein.

Tables 2-4 provide GO-pathway analysis from RNA seq study.

TABLE 2
Molecular pathway analysis; CLA
vs Control (all genotypes). Analyzed
via MouseMine: http://www.mousemine.org/mousemine/begin.do.

| | Adjusted p-Value |
|---|---|
| | (Holm-Bonferroni) |
| Gene Ontology Enrichment-Biological Process | |
| Cellular metabolic process [GO:0044237] | 0.001532 |
| rRNA processing [GO:0006364] | 0.008305 |
| Organophosphate metabolic | 0.008949 |
| process [GO:0019637] | |
| rRNA metabolic process | 0.015735 |
| [GO:0016072] | |
| Cellular component organization | 0.020753 |
| or biogenesis [GO:0071840] | |
| Gene Ontology Enrichment-Anatomy | |
| Telencephalon | 0.016790 |
| Retina | 0.038596 |
| Eye posterior segment | 0.039956 |
| Peripheral nervous system | 0.042822 |
| Gene Ontology Enrichment-Mammalian Phenotype | |
| Mortality/aging [MP:0010768] | 6.55E−06 |
| Preweaning lethality [MP:0010770] | 1.40E−05 |
| Abnormal survival [MP:0010769] | 2.12E−05 |
| Embryonic lethality prior to | 6.05E−05 |
| tooth bud stage [MP:0013293] | |
| Prenatal lethality [MP:0002080] | 1.84E−04 |
| Embryonic lethality [MP:0008762] | 1.85E−04 |
| Embryonic lethality prior | 2.56E−04 |
| to organogenesis [MP:0013292] | |
| Abnormal behavior [MP:0004924] | 0.029506 |
| Behavior/neurological | 0.029506 |
| phenotype [MP:0005386] | |

TABLE 3
Molecular pathway analysis; CLA vs
Control (WT genotype only). Analyzed
via MouseMine: http://www.mousemine.org/mousemine/begin.do.

| Gene Ontology Enrichment- | Adjusted p-Value |
|---|---|
| Mammalian Phenotype | (Holm-Bonferroni) |
| Cellular response to starvation | 0.007261 |
| [GO:0009267] | |
| Cellular responses to stress | 0.01229 |
| Cellular responses to stimuli | 0.01295 |
| Response to starvation | 0.015332 |
| [GO:0042594] | |
| Cellular response to nutrient | 0.020484 |
| levels [GO:0031669] | |

TABLE 4
Molecular pathway analysis; PWS (Snord116m+/p−) versus
WT (both diets). Analyzed via MouseMine:
http://www.mousemine.org/mousemine/begin.do.

| Gene Ontology Enrichment- | Adjusted p-Value |
|---|---|
| Mammalian Phenotype | (Holm-Bonferroni) |
| RBM3/CIRBP, RNA recognition | 0.020798 |
| [IPR034278] | |
| Abnormal bone mineral | 0.046985 |
| content [MP:0010122] | |

### 3.11 Statistical Data

Statistical analysis for Tables 5-28 was performed using JMP Statistical Software. Version Pro15. underline=significant, double underline=P less than 0.0001.

TABLE 5
Body weight by week

| | | | Sum of | F | Prob > |
|---|---|---|---|---|---|
| Source | Nparm | DF | Squares | ratio | F |
| Genotype | 2 | 2 | 2415.0555 | 234.1928 | <0.0001* |
| Treatment | 1 | 1 | 926.1966 | 179.6303 | <0.0001* |
| Week | 12 | 12 | 541.3119 | 8.7487 | <0.0001* |
| Genotype*Treatment | 2 | 2 | 46.2957 | 4.4894 | 0.0117* |
| Genotype*Week | 24 | 24 | 48.7914 | 0.3943 | 0.9961 |
| Treatment*Week | 12 | 12 | 150.5177 | 2.4327 | 0.0044* |
| Genotype*Treat- | 24 | 24 | 58.8984 | 0.4760 | 0.9847 |
| ment*Week | | | | | |

TABLE 6
Body weight - week 12

| | | | Sum of | F | |
|---|---|---|---|---|---|
| Source | Nparm | DF | Squares | ratio | Prob > F |
| Genotype | 2 | 2 | 241.28281 | 17.3889 | <0.0001* |
| Treatment | 1 | 1 | 162.47934 | 23.4193 | <0.0001* |
| Genotype*Treatment | 2 | 2 | 12.09191 | 0.8714 | 0.4259 |

TABLE 7
Fat Mass by week

| | | | Sum of | F | Prob > |
|---|---|---|---|---|---|
| Source | Nparm | DF | Squares | ratio | F |
| Genotype | 2 | 2 | 153.72562 | 109.7742 | <0.0001* |
| Treatment | 1 | 1 | 433.45607 | 619.0547 | <0.0001* |
| Week | 12 | 12 | 18.02313 | 2.1450 | 0.0132* |
| Genotype*Treatment | 2 | 2 | 45.68598 | 32.6240 | <0.0001* |
| Genotype*Week | 24 | 24 | 10.97833 | 0.6533 | 0.8962 |
| Treatment*Week | 12 | 12 | 105.09223 | 12.5076 | <0.0001* |
| Genotype*Treat- | 24 | 24 | 19.49379 | 1.1600 | 0.2733 |
| ment*Week | | | | | |

TABLE 8
Fat Mass - week 12

| | | | Sum of | F | Prob > |
|---|---|---|---|---|---|
| Source | Nparm | DF | Squares | ratio | F |
| Genotype | 2 | 2 | 17.470671 | 9.6695 | 0.0004* |
| Treatment | 1 | 1 | 83.374886 | 92.2914 | <0.0001* |
| Genotype*Treatment | 2 | 2 | 9.416571 | 5.2118 | 0.0096* |

TABLE 9
Lean Body Mass by Week

| | | | Sum of | F | Prob > |
|---|---|---|---|---|---|
| Source | Nparm | DF | Squares | ratio | F |
| Genotype | 2 | 2 | 375.88168 | 52.9455 | <0.0001* |
| Treatment | 1 | 1 | 0.01110 | 0.0031 | 0.9554 |
| Week | 12 | 12 | 481.39952 | 11.3014 | <0.0001* |
| Genotype*Treatment | 2 | 2 | 34.13439 | 4.8081 | 0.0085* |

TABLE 10
Lean Body Mass - week 12

| | | | Sum of | F | Prob > |
|---|---|---|---|---|---|
| Source | Nparm | DF | Squares | ratio | F |
| Genotype | 2 | 2 | 101.94217 | 15.2837 | <0.0001* |
| Treatment | 1 | 1 | 2.75841 | 0.8271 | 0.3684 |
| Genotype*Treatment | 2 | 2 | 1.30931 | 0.1963 | 0.8225 |

TABLE 11
Temperature - by week

| | | | Sum of | F | Prob > |
|---|---|---|---|---|---|
| Source | Nparm | DF | Squares | ratio | F |
| Genotype | 2 | 2 | 1.108770 | 1.2263 | 0.2942 |
| Treatment | 1 | 1 | 1.180395 | 2.6110 | 0.1067 |
| Week | 12 | 12 | 10.030372 | 1.8489 | 0.0382* |
| Genotype*Treatment | 2 | 2 | 0.010462 | 0.0116 | 0.9885 |
| Genotype*Week | 24 | 24 | 6.518275 | 0.6007 | 0.9341 |
| Treatment*Week | 12 | 12 | 4.271536 | 0.7874 | 0.6638 |
| Genotype*Treat- | 24 | 24 | 7.420291 | 0.6839 | 0.8693 |
| ment*Week | | | | | |

TABLE 12
Temperature - week 12

| | | | Sum of | F | Prob > |
|---|---|---|---|---|---|
| Source | Nparm | DF | Squares | ratio | F |
| Genotype | 2 | 2 | 0.7213672 | 0.9420 | 0.3891 |
| Treatment | 1 | 1 | 0.0514747 | 0.1344 | 0.7158 |
| Genotype*Treatment | 2 | 2 | 1.5644544 | 2.0429 | 0.1426 |

TABLE 13
Temperature - whole study

| | | | Sum of | F | Prob > |
|---|---|---|---|---|---|
| Source | Nparm | DF | Squares | ratio | F |
| Genotype | 2 | 2 | 1.894437 | 2.2221 | 0.1094 |
| Treatment | 1 | 1 | 2.026500 | 4.7541 | 0.0297* |
| Week | 12 | 12 | 10.392803 | 2.0318 | 0.0200* |
| Genotype*Treatment | 2 | 2 | 0.283136 | 0.3321 | 0.7176 |
| Genotype*Week | 24 | 24 | 8.144273 | 0.7961 | 0.7435 |
| Treatment*Week | 12 | 12 | 3.555686 | 0.6951 | 0.7568 |
| Genotype*Treat- | 24 | 24 | 6.938580 | 0.6782 | 0.8745 |
| ment*Week | | | | | |

TABLE 14
Food Intake - by week

| | | | Sum of | F | Prob > |
|---|---|---|---|---|---|
| Source | Nparm | DF | Squares | ratio | F |
| Genotype | 2 | 2 | 972.0919 | 41.9005 | <0.0001* |
| Treatment | 1 | 1 | 67.7568 | 5.8411 | 0.0160* |
| Week | 11 | 11 | 1271.8840 | 9.9678 | <0.0001* |
| Genotype*Treatment | 2 | 2 | 21.9269 | 0.9451 | 0.3893 |
| Genotype*Week | 22 | 22 | 123.1850 | 0.4827 | 0.9785 |
| Treatment*Week | 11 | 11 | 63.8593 | 0.5005 | 0.9031 |
| Genotype*Treat- | 22 | 22 | 150.2563 | 0.5888 | 0.9319 |
| ment*Week | | | | | |

TABLE 15
Food Intake/Body Weight, by week

| | | | Sum of | F | Prob > |
|---|---|---|---|---|---|
| Source | Nparm | DF | Squares | ratio | F |
| Genotype | 2 | 2 | 0.348 | 7.58 | <0.00006* |
| Treatment | 1 | 1 | 1.236 | 53.768 | <0.0001* |
| Week | 11 | 11 | 4.000 | 15.810 | <0.0001* |
| Genotype*Treatment | 2 | 2 | 0.058 | 1.275 | 0.2802 |
| Genotype*Week | 22 | 22 | 0.205 | 0.406 | 0.9931 |
| Treatment*Week | 11 | 11 | 0.1433 | 0.5664 | 0.8563 |
| Genotype*Treat- | 22 | 22 | 0.3670 | 0.725 | 0.815 |
| ment*Week | | | | | |

TABLE 16
Food Intake - week 12

| | | | Sum of | F | Prob > |
|---|---|---|---|---|---|
| Source | Nparm | DF | Squares | ratio | F |
| Genotype | 2 | 2 | 73.062538 | 2.4121 | 0.1029 |
| Treatment | 1 | 1 | 0.015142 | 0.0010 | 0.9749 |
| Genotype*Treatment | 2 | 2 | 12.499807 | 0.4127 | 0.6647 |

TABLE 17
Fasting Glucose-Pre

| | | | Sum of | F | |
|---|---|---|---|---|---|
| Source | Nparm | DF | Squares | ratio | Prob > F |
| Genotype | 2 | 2 | 4962.6770 | 4.2635 | 0.0202* |

TABLE 18
Fasting Glucose-Post

| | | | Sum of | F | |
|---|---|---|---|---|---|
| Source | Nparm | DF | Squares | ratio | Prob > F |
| Genotype | 2 | 2 | 2329.489 | 0.7440 | 0.4815 |
| Treatment | 1 | 1 | 11718.166 | 7.4856 | 0.0092* |
| Genotype*Treatment | 2 | 2 | 2002.489 | 0.6396 | 0.5327 |

TABLE 19
Area under the Curve (Glucose tolerance)-Pre

| | | | Sum of | F | |
|---|---|---|---|---|---|
| Source | Nparm | DF | Squares | ratio | Prob > F |
| GENOTYPE | 2 | 2 | 203267705 | 4.21907 | 0.0217* |

TABLE 20
Area under the Curve (Glucose tolerance)-Pre

| | | | Sum of | F | |
|---|---|---|---|---|---|
| Source | Nparm | DF | Squares | ratio | Prob > F |
| Genotype | 2 | 2 | 76328543 | 0.5721 | 0.5689 |
| Treatment | 1 | 1 | 191679715 | 2.8731 | 0.0978 |
| Genotype*Treatment | 2 | 2 | 203031326 | 1.5216 | 0.2307 |

TABLE 21
Metabolism RER post (only)

| | | | Sum of | F | |
|---|---|---|---|---|---|
| Source | Nparm | DF | Squares | ratio | Prob > F |
| Genotype | 2 | 2 | 0.00697954 | 2.0608 | 0.1469 |
| Treatment | 1 | 1 | 0.00003781 | 0.0223 | 0.8823 |
| Genotype*Treatment | 2 | 2 | 0.00016326 | 0.0482 | 0.9530 |

TABLE 22
Metabolism EE post (only)

| | | | Sum of | F | |
|---|---|---|---|---|---|
| Source | Nparm | DF | Squares | ratio | Prob > F |
| Genotype | 2 | 2 | 683.29358 | 0.9195 | 0.4106 |
| Treatment | 1 | 1 | 813.65946 | 2.1898 | 0.1505 |
| Genotype*Treatment | 2 | 2 | 434.79191 | 0.5851 | 0.5640 |

TABLE 23
Area under the Curve (Glucose tolerance)-Pre

| | | | Sum of | F | |
|---|---|---|---|---|---|
| Source | Nparm | DF | Squares | ratio | Prob > F |
| Genotype | 2 | 2 | 5853024.7 | 3.1810 | 0.0510 |

TABLE 24
Running Wheels-Post

| | | | Sum of | F | |
|---|---|---|---|---|---|
| Source | Nparm | DF | Squares | ratio | Prob > F |
| Genotype | 2 | 2 | 40847854 | 9.3478 | 0.0005* |
| Treatment | 1 | 1 | 2062316 | 0.9439 | 0.3370 |
| Genotype*Treatment | 2 | 2 | 17828236 | 4.0799 | 0.0242* |

TABLE 25
24-hr Home Cage Activity-post (only)

| | | | Sum of | F | |
|---|---|---|---|---|---|
| Source | Nparm | DF | Squares | ratio | Prob > F |
| Genotype | 2 | 2 | 32341.628 | 2.2751 | 0.1221 |
| Treatment | 1 | 1 | 8607.440 | 1.2110 | 0.2808 |
| Genotype*Treatment | 2 | 2 | 12419.832 | 0.8737 | 0.4289 |

TABLE 26
Rotorod-post (only)

| | | | Sum of | F | |
|---|---|---|---|---|---|
| Source | Nparm | DF | Squares | ratio | Prob > F |
| Genotype | 2 | 2 | 10255.044 | 2.7049 | 0.0788 |
| Treatment | 1 | 1 | 5714.448 | 3.0146 | 0.0900 |
| Genotype*Treatment | 2 | 2 | 3718.096 | 0.9807 | 0.3837 |

TABLE 27
Elevated Plus - open arms-Pre

| | | | Sum of | F | |
|---|---|---|---|---|---|
| Source | Nparm | DF | Squares | ratio | Prob > F |
| Genotype | 2 | 2 | 3355.1057 | 0.7831 | 0.4635 |
| Treatment | 1 | 1 | 3042.2047 | 1.4202 | 0.2401 |
| Genotype*Treatment | 2 | 2 | 3979.5834 | 0.9829 | 0.4030 |

TABLE 28
Elevated Plus - open arms-Post

| | | | Sum of | F | |
|---|---|---|---|---|---|
| Source | Nparm | DF | Squares | ratio | Prob > F |
| Genotype | 2 | 2 | 13845.656 | 2.8814 | 0.0675 |
| Treatment | 1 | 1 | 2.274 | 0.0009 | 0.9756 |
| Genotype*Treatment | 2 | 2 | 2594.691 | 0.5400 | 0.5896 |

Statistical analysis Tables 29-31 are from GraphPad Prism 9.2.0. Underline=significant. Double underline=P less than 0.0001.

TABLE 29
Torque normalized to body mass (end of study)

| | | Sum of | F | |
|---|---|---|---|---|
| Source | DF | Squares | ratio | Prob > F |
| Group | 5 | 0.2844 | 16.42 | <0.0001* |
| Frequency | 10 | 13.18 | 380.6 | <0.0001* |
| Interaction | 50 | 0.07779 | 0.4491 | 0.9996 |

TABLE 30
Torque normalized to lean body mass (end of study)

| | | Sum of | F | |
|---|---|---|---|---|
| Source | DF | Squares | ratio | Prob > F |
| Group | 5 | 0.1823 | 7.063 | <0.0001* |
| Frequency | 10 | 21.45 | 415.7 | <0.0001* |
| Interaction | 50 | 0.1424 | 0.5520 | 0.9945 |

TABLE 31
Fatigue as percent of initial peak contractile torque (end of study)

| | | Sum of | F | |
|---|---|---|---|---|
| Source | DF | Squares | ratio | Prob > F |
| Group | 5 | 15948 | 30.46 | <0.0001* |
| Frequency | 29 | 364825 | 120.1 | <0.0001* |
| Interaction | 145 | 3708 | 0.2442 | >0.9999 |

### 4. Discussion

Obesity and the complications of increased body weight and fat mass represent one of the main adult phenotypes impacting health and well-being for patients with PWS. The main goal of the study was to determine if CLA treatment would result in body weight and fat loss in mice with a single or double deletion of Snord116 (PWS, or PWS-KO mice, respectively). The Snord116 locus encodes multiple small nucleolar RNAs and is one of the three genes within the minimal causative genomic region for PWS [43]. Mice with a single paternal deletion of Snord116, as well as a deletion of both alleles of Snord116, were included in this study. These findings demonstrate that deletion of Snord116 either hemi-or homozygously does not interfere with the weight- and fat-reducing properties of CLA. Despite the lack of overt obesity of the PWS and PWS-KO mice, even on a 20% fat diet, both genotypes showed significant body weight loss due to body fat and not lean (muscle) mass loss. Of note, Tonalin™, which is a 50:50 mixture of CLA isoform, has mixed results overall in weight reduction for humans, perhaps due to the wide variability in dosage per gram body mass given in each study [17]. In humans, studies achieving weight loss were dosing at a minimum of 2.8 g/day/kg body mass for at least 4 months. In this study, a Tonalin™ CLA dosing was calculated to be at an equivalent dose of 5.2 g CLA/kg body mass in free-feeding animals (0.13 g total per day). A study of 73 patients (age range 16-58 years), with either deletion of the locus (64%) or uniparental disomy (36%), the average body mass was 99.4 kg and 81.0 kg, respectively, and in the overweight-to-obese range for those individuals [51]. Thus, for an adult individual with PWS at ˜90 kg, a daily dosage of 468 g of CLA would be equivalent in grams CLA/kg body mass. Obviously, this is not achievable or within GRAS guidelines, so dosage of patients with PWS would have to be titrated to find the optimal range for body weight loss.

Patients with PWS experience increased body weight due to both hyperphagia and reduced physical activity [43,52,53]. There is also a documented reduction in overall energy expenditure in these patients [52]. In examining the possible mechanisms of action for CLA treatment, there does not seem to be any reduction in food intake with CLA intake, either in this study or in other reports in both mice (e.g., [54]) and in overweight humans (e.g., [55]), although results are mixed (e.g., [56]). In the study by Kamphius and colleagues [55], feelings of fullness and satiety were increased and hunger was decreased with CLA supplementation. This could be an important consideration for patients, perhaps giving the ability to eat regularly, rather than diet, or could work in conjunction with a low-fat, low-calorie diet regimen. Habitual physical activity levels are significantly lower in patients with PWS compared to both controls with and without obesity [57]. It was anticipated that, like N2KO mice treated with CLA [14-16], PWS mice would demonstrate increased 24 h spontaneous wheel running with CLA treatment compared to control. However, while there was a significant effect of genotype on running wheel activity prior to the intervention, there were no differences in genotype post-treatment. Likewise, home-cage activity, as measured in the TSE apparatus, was not different for the effect of either genotype or treatment.

Patients with PWS display overall muscle hypotonia and functional weakness. Several studies report reduced cross-sectional area and unusual morphology of PWS-affected muscles [58,59]. Few studies have quantified muscle strength in PWS patients [59,60]. Females with PWS showed lower-knee extensor and flexor torque than both obese and non-obese controls [60]. When plantar-flexor muscle function was measured in adults with PWS, lower absolute peak torque output was produced than controls with obesity [59]. When normalized to cross-sectional area, there were no differences in torque between PWS patients and controls [59]. Similarly, the present study demonstrates that PWS and PWS-KO mice do not display plantar-flexor weakness, with or without CLA treatment. In healthy subjects, CLA supplementation in combination with resistance training has been reported to better maintain lean body mass and improve some metrics of strength in humans [38]. Similarly, a study in older WT mice fed a high-fat diet and supplemented with a combination of CLA and omega-3 increased grip strength and improved body composition after performing resistance training [61]. However, a control group from the same study that did not perform resistance training but did receive CLA and omega-3 supplementation displayed lowered grip strength and cross-sectional area of muscle [61]. These data indicate that CLA supplementation was beneficial to torque output in WT mice. In addition, PWS and PWS-KO mice did not show decreased torque output in comparison to WT mice, regardless of normalization type or CLA treatment. CLA treatment lowered torque output in the PWS group normalized to lean body mass. Resistance to fatigue was also lowered in PWS CLA and PWS-KO CLA groups compared to their control diet counterparts. However, CLA does not lower any of the torque measurements below WT. Therefore, CLA is not detrimental to muscle function, but does provide favorable changes in body composition.

Several studies have demonstrated that CLA can cause browning of white adipose tissue (e.g., [62]). As brown adipose tissue is important for heat generation, this would be expected to increase body temperature. Interestingly, in the mice on the CLA diet, body temperature dropped significantly regardless of genotype, and this is consistent with another study where mice on CLA diet exposed to cold had significant drops in body temperature compared to control mice [63]. Thus, it appears that body temperature may be more responsive to the loss of insulation from the loss of white adipose than any potential browning of the remaining white adipose in the CLA-treated mice. Metabolic analysis revealed that both respiratory exchange ratio and metabolic rate were unaffected by genotype or treatment, although there did appear to be a higher metabolic rate with CLA pre- versus post-treatment, similar to that seen in mice treated with CLA (e.g., [64]) and in some human studies ([17]). As there was a failure of the TSE system during the study, it is possible that the data would have shown significant increases in metabolic rate if all animals in the study had been included in the metabolism analysis. These data suggest that CLA treatment is having some non-food intake, non-exercise effect on body weight, consistent with the increased (although non-significant) effect on whole-body energy expenditure. Any future study of CLA treatment of PWS patients in humans should consider overall body metabolism analysis to be an important measurement.

In further attempting to characterize physiological differences in CLA-treated PWS and PWS-KO mice, fasting glucose and glucose tolerance tests, as well as liver histology, were measured. Not unexpectedly, CLA treatment significantly increased fasting glucose, and overall AUC in GTT tests, regardless of genotype. Likewise, CLA treatment increases the incidence of liver steatosis regardless of genotype. Several reports have demonstrated that it is the t10, c12 CLA isomer that is responsible for liver steatosis development, and that the mechanism appears to be due to increased circulating free fatty acids and glucose that are picked up by the liver, and not adequately oxidized [65]. Proteomic and molecular analyses of fatty liver in mice treated with CLA have shown increases in gene expression and levels of proteins related to lipogenesis [65,66]. In this Example, 100% of WT mice demonstrated liver steatosis on CLA diet, but only ˜50% of PWS and PWS-KO mice did. While there is mixed agreement as to whether CLA would have similar effects on hepatic steatosis in humans [17], these results demonstrate that the PWS genotype may protect against this condition, but more work would be needed to understand the mechanisms occurring.

Applicant undertook an RNA-seq approach to attempt to identify Snord116-deletion-specific effects and CLA-specific effects on mRNA expression in the hypothalamus, possibly linking these to relevant pathways. This is the first RNA-seq study examining the hypothalamus in mice with a CLA-supplemented diet, to the authors' knowledge [5,6,14-16,40,43]. In total, 580 genes were differentially expressed using an FDR of 0.10 and most of these only had modestly changed RNA levels between 0.5- and 2-fold. The gene with the most differential expression was Ipw, a non-coding RNA gene that is deleted in both PWS patients and the PWS mouse [43], so this finding was expected and confirmed the overall differential expression analysis. Significant gene ontology pathways for control diet versus CLA diet of nutrient and stress responses were unsurprising but may still yield future information on the mechanism of action of CLA in the hypothalamus. Interestingly, one gene, Mael, was “rescued” by CLA in PWS mice. Mael plays a central role in spermatogenesis [67], but the overall expression level in hypothalamus was very low, making it difficult to assess any biological relevance of this finding. Only one gene was confirmed to be differentially regulated between control and CLA treatments (all genotypes) by secondary qPCR analysis, Chmp1b. The protein product of Chmp1b is an endosomal-associated protein involved in the endosomal sorting complexes required for transport (ESCRT) that may be involved in the multi-vesicular body (MVB), a specialized endosome [68-70]. Chmp1b protein has been implicated to support lipid-droplet to peroxisome fatty-acid trafficking [71]. As Chmp1b was increased with CLA diet, these data suggest there may be more membrane protein turnover or fatty-acid oxidation with CLA. However, more studies are needed to confirm this finding. The RBM3/CIRBP RNA binding protein GO pathway, which was significant for WT versus PWS mice, includes Rbm3 and Cirbp, which are two RNA binding proteins induced by hypothermia, and modulated during circadian rhythms [72]. This is an interesting finding as both PWS mice and individuals with PWS have altered circadian rhythms and sleep cycles [73]. The two RNA binding proteins are thought to control polyadenylation of mRNAs, changing post-transcriptional mRNA stability. This finding links Applicant's more recent finding that Nhlh2 mRNA stability is modulated by Snord116, possibly through a motif that is very near to the end of the transcript [5], as well as Applicant's previous data showing Nhlh2 mRNA oscillation with cold-temperature exposure [74]. Future studies to examine this pathway may help to clarify any relationship between downregulation of Nhlh2 in PWS and hypothalamic circadian and sleep patterns. Finally, for WT versus PWS mice (all treatments) two genes were further analyzed by qPCR, but only one, the Gagbr2 gene encoding GABA receptor Type A, subunit gamma2, was significantly upregulated in PWS mice with secondary analysis. In the PVN of the hypothalamus, Gabrg2 protein has been implicated in diurnal rhythmicity in metabolism and diet-induced obesity through upstream regulation by the circadian gene Bmall [75]. Loss of Gabrg2 in the PVN leads to obesity and loss of diurnal rhythm of energy expenditure and food intake [75]. Additionally, Gabrg2 is diurnally regulated in the suprachiasmatic nucleus (SCN) of hamster and mice [76]. Interestingly, GABA neurotransmitter levels are reduced in individuals with PWS, and it is thought that this reduction can lead to some of the neurodevelopmental and behavioral problems with those patients [77]. Upregulation of one of the receptors for GABA, a major neuro-inhibitory neurotransmitter could be secondary to the reduced GABA brain levels, possibly contributing to the overall GABAergic dysfunction. Interestingly, in patients who have PWS as a result of the large deletion on 15q, other forms of the GABA A receptor can be included in the deletion [78]. Of note, Applicant failed to find differential expression of some genes implicated in hypothalamus dysfunction of PWS mice, including Nhlh2, Pomc, Npy, and others. Without being bound by theory, Applicant hypothesized that a cell-type specific approach under specific signaling conditions may better capture sensitivity to Snord116-mediated regulation. Overall, the mRNA-seq and follow-up RT-QPCR show very modest changes, if any. The weak effect size for all differentially expressed genes in the current RNA-seq study also leads to difficulty in validation of differentially expressed genes, as RT-QPCR is limited by the sample size in the current study and their natural variability. Similarly, previous RNA expression studies of hypothalamus tissue from PWS mouse models have shown modest effect sizes [8,11,79-81].

As this was a dietary intervention, microbiome analysis was performed to determine if CLA diet, the PWS (Snord116m+/p−) genotype, or the interaction of these could have effects on gut microbiota. Previous analyses of individuals with PWS have found that those individuals with obesity share a similar microbiome compared to individuals with non-syndromic obesity [82,83], and that a dietary intervention that modulated obesity had similar effects on the microbiome, namely, an increase in Bifidobacterium sp. [83]. PWS and PWS-KO mice also shared a similar microbiome with WT mice (i.e., no genotype differences). These findings are somewhat in contrast to a 2021 study showing that in-dividuals with PWS had significantly fewer Bifidobacterium sp. than others in the study, including individuals with irritable bowel syndrome [84]. However, in that same study, levels of Tenericutes were significantly higher in those individuals with PWS, which was not replicated in the mouse model herein. Turicibacter sp. decreased in CLA diet, and while not much is published on this genus, it was also found to be low in Type I diabetic children [85]. Relative abundance of Bacteriodetes and Firmicutes showed the trend towards decrease and increase (non-significant), respectively, in PWS and PWS-KO compared to WT mice. This is consistent with overall Bacteriodetes/Firmicutes profile in obese vs. normal-weight individuals. Firmicutes are known for their energy harvesting capabilities and tend to show increase in obese individuals [86]. The microbiome field has been explored to great length in many studies of late, however, the consensus on its being causative or merely associative is still debated in many of them. In this Example, the modest changes observed in microbiome composition in the PWS model, are not per se causative, but show an a-sociation. Before the microbiome can be placed as one of the mechanisms, microbiome-derived metabolites, secondary bile acids, and the whole metabolome need to be studied. However, the microbiome aspect of the current Example opens up a new area of investigation in the unique mouse models Applicant used in this Example.

Overall, the original hypothesis that CLA diet would reduce body weight and body fat in mice carrying a paternally inherited deletion of Snord116 was confirmed. Consistent with the literature on CLA diet, there was no detectable change in food intake throughout the 12-week study. While fasting glucose was, as expected, increased with CLA diet in all genotypes, there was an unexpected difference in hepatic steatosis, with about half of the PWS and PWS-KO genotype mice not showing fatty liver, compared to all of the WT mice on CLA diet. This difference appears to be genotype-specific, although more work is needed to determine the mechanism. Interestingly, neither lean mass nor muscle function were compromised or improved by CLA treatment. Metabolism was also unchanged, leading us to be unable to conclude the mechanism for CLA-induced body fat loss. Studies that were designed to tease out CLA-induced or -suppressed pathways through either mRNA regulation in the hypothalamus or microbiome analysis in the gut have provided intriguing leads for future studies.

### REFERENCES FOR EXAMPLE 1

1. Chen, H.; Victor, A. K.; Klein, J.; Tacer, K. F.; Tai, D. J.; de Esch, C.; Nuttle, A.; Temirov, J.; Burnett, L. C.; Rosenbaum, M.; et al. Loss of MAGEL2 in Prader-Willi syndrome leads to decreased secretory granule and neuropeptide production. JCI Insight 2020, 5, e138576. [CrossRef] [PubMed]
2. Tan, Q.; Potter, K. J.; Burnett, L. C.; Orsso, C. E.; Inman, M.; Ryman, D. C.; Haqq, A. M. Prader-Willi-Like Phenotype Caused by an Atypical 15q11.2 Microdeletion. Genes 2020, 11, 128. [CrossRef]
3. Burnett, L. C.; LeDuc, C. A.; Sulsona, C. R.; Paull, D.; Rausch, R.; Eddiry, S.; Carli, J. F.; Morabito, M. V.; Skowronski, A. A.; Hubner, G.; et al. Deficiency in prohormone convertase PC1 impairs prohormone processing in Prader-Willi syndrome. J. Clin. Investig. 2017, 127, 293-305. [CrossRef] [PubMed]
4. Bittel, D. C.; Kibiryeva, N.; Sell, S. M.; Strong, T. V.; Butler, M. G. Whole genome microarray analysis of gene expression in Prader-Willi syndrome. Am. J. Med. Genet. A 2007, 143, 430-442. [CrossRef] [PubMed]
5. Kocher, M. A.; Huang, F. W.; Le, E.; Good, D. J. Snord116 Post-transcriptionally Increases Nhlh2 mRNA Stability: Implications for Human Prader-Willi Syndrome. Hum. Mol. Genet. 2021, 30, 1101-1110. [CrossRef] [PubMed]
6. Good, D. J.; Porter, F. D.; Mahon, K. A.; Parlow, A. F.; Westphal, H.; Kirsch, I. R. Hypogonadism and obesity in mice with a targeted deletion of the Nhlh2 gene. Nat. Genet. 1997, 15, 397-401. [CrossRef]
7. Ding, F.; Li, H. H.; Zhang, S.; Solomon, N. M.; Camper, S. A.; Cohen, P.; Francke, U. SnoRNA Snord116 (Pwcrl/MBII-85) deletion causes growth deficiency and hyperphagia in mice. PLOS ONE 2008, 3, e1709. [CrossRef]
8. Polex-Wolf, J.; Lam, B. Y.; Larder, R.; Tadross, J.; Rimmington, D.; Bosch, F.; Cenzano, V. J.; Ayuso, E.; Ma, M. K.; Rainbow, K.; et al. Hypothalamic loss of Snord116 recapitulates the hyperphagia of Prader-Willi syndrome. J. Clin. Investig. 2018, 128, 960-969. [CrossRef]
9. Kummerfeld, D. M.; Raabe, C. A.; Brosius, J.; Mo, D.; Skryabin, B. V.; Rozhdestvensky, T. S. A Comprehensive Review of Genetically Engineered Mouse Models for Prader-Willi Syndrome Research. Int. J. Mol. Sci. 2021, 22, 3613. [CrossRef]
10. Eddiry, S.; Dienne, G.; Molinas, C.; Salles, J.; Auriol, F. C.; Gennero, I.; Bieth, E.; Skryabin, B. V.; Rozhdestvensky, T. S.; Burnett, L. C.; et al. SNORD116 and growth hormone therapy impact IGFBP7 in Prader-Willi syndrome. Genet. Med. 2021, 23, 1664-1672. [CrossRef]
11. Qi, Y.; Purtell, L.; Fu, M.; Lee, N. J.; Aepler, J.; Zhang, L.; Loh, K.; Enriquez, R. F.; Baldock, P. A.; Zolotukhin, S.; et al. Snord116 is critical in the regulation of food intake and body weight. Sci. Rep. 2016, 6, 18614. [CrossRef] [PubMed]
12. Pellikaan, K.; van Woerden, G. M.; Kleinendorst, L.; Rosenberg, A. G. W.; Horsthemke, B.; Grosser, C.; van Zutven, L.; van Rossum, E. F. C.; van der Lely, A. J.; Resnick, J. L.; et al. The Diagnostic Journey of a Patient with Prader-Willi-Like Syndrome and a Unique Homozygous SNURF-SNRPN Variant; Bio-Molecular Analysis and Review of the Literature. Genes 2021, 12, 875. [CrossRef]
13. Hur, S.; Whitcomb, F.; Rhee, S.; Park, Y.; Good, D. J.; Park, Y. Effects of trans-10, cis-12 conjugated linoleic acid on body composition in genetically obese mice. J. Med. Food 2009, 12, 56-63. [CrossRef] [PubMed]
14. Kim, J. H.; Gilliard, D.; Good, D. J.; Park, Y. Preventive effects of conjugated linoleic acid on obesity by improved physical activity in nescient basic helix-loop-helix 2 knockout mice during growth period. Food Funct. 2012, 3, 1280-1285. [CrossRef] [PubMed]
15. Kim, J. H.; Park, Y.; Kim, D.; Good, D. J.; Park, Y. Dietary conjugated nonadecadienoic acid prevents adult-onset obesity in nescient basic helix-loop-helix 2 knockout mice. J. Nutr. Biochem. 2013, 24, 556-566. [CrossRef]
16. Kim, Y.; Kim, D.; Good, D. J.; Park, Y. Effects of postweaning administration of conjugated linoleic acid on development of obesity in nescient basic helix-loop-helix 2 knockout mice. J. Agric. Food Chem. 2015, 63, 5212-5223. [CrossRef]
17. Kim, J. H.; Kim, Y.; Kim, Y. J.; Park, Y. Conjugated Linoleic Acid: Potential Health Benefits as a Functional Food Ingredient. Annu. Rev. Food Sci. Technol. 2016, 7, 221-244. [CrossRef]
18. Yang, X. Growth hormone treatment for Prader-Willi syndrome: A review. Neuropeptides 2020, 83, 102084. [CrossRef]
19. Hogg, S. A review of the validity and variability of the elevated plus-maze as an animal model of anxiety. Pharmacol. Biochem. Behav. 1996, 54, 21-30. [CrossRef]
20. Coyle, C. A.; Jing, E.; Hosmer, T.; Powers, J. B.; Wade, G.; Good, D. J. Reduced voluntary activity precedes adult-onset obesity in Nhlh2 knockout mice. Physiol. Behav. 2002, 77, 387-402. [CrossRef]
21. Weir, J. B. New methods for calculating metabolic rate with special reference to protein metabolism. J. Physiol. 1949, 109, 1-9. [CrossRef] [PubMed]
22. Hamm, S. E.; Fathalikhani, D. D.; Bukovec, K. E.; Addington, A. K.; Zhang, H.; Perry, J. B.; McMillan, R. P.; Lawlor, M. W.; Prom, M. J.; Vanden Avond, M. A.; et al. Voluntary wheel running complements microdystrophin gene therapy to improve muscle function in mdx mice. Mol. Ther. Methods Clin. Dev. 2021, 21, 144-160. [CrossRef] [PubMed]
23. Love, M. I.; Huber, W.; Anders, S. Moderated estimation of fold change and dispersion for RNA-seq data with DESeq2. Genome Biol. 2014, 15, 550. [CrossRef] [PubMed]
24. Love, M. I.; Huber, W.; Ander, S. Differential Analysis of Count Data—The DESeq2 Package. 2016. Available online: https://citeseerx.ist.psu.edu/viewdoc/summary?doi=10.1.1.733.1832 (accessed on 29 Sep. 2021).
25. Love, M.; Anders, S.; Huber, W. Differential analysis of count data—The DESeq2 package-4 Feb. 2014. Genome Biol. 2014, 15, 10-1186.
26. Li, Y.; Andrade, J. DEApp: An interactive web interface for differential expression analysis of next generation sequence data. Source Code Biol. Med. 2017, 12, 2. [CrossRef]
27. Motenko, H.; Neuhauser, S. B.; O'Keefe, M.; Richardson, J. E. MouseMine: A new data warehouse for MGI. Mamm. Genome 2015, 26, 325-330. [CrossRef]
28. Kozich, J. J.; Westcott, S. L.; Baxter, N. T.; Highlander, S. K.; Schloss, P. D. Development of a dual-index sequencing strategy and curation pipeline for analyzing amplicon sequence data on the MiSeq Illumina sequencing platform. Appl. Environ. Microbiol. 2013, 79, 5112-5120. [CrossRef]
29. Bolyen, E.; Rideout, J. R.; Dillon, M. R.; Bokulich, N. A.; Abnet, C. C.; Al-Ghalith, G. A.; Alexander, H.; Alm, E. J.; Arumugam, M.; Asnicar, F.; et al. Reproducible, interactive, scalable and extensible microbiome data science using QIIME 2. Nat. Biotechnol. 2019, 37, 852-857. [CrossRef]
30. Bokulich, N. A.; Subramanian, S.; Faith, J. J.; Gevers, D.; Gordon, J. I.; Knight, R.; Mills, D. A.; Caporaso, J. G. Quality-filtering vastly improves diversity estimates from Illumina amplicon sequencing. Nat. Methods 2013, 10, 57-59. [CrossRef]
31. Amir, A.; McDonald, D.; Navas-Molina, J. A.; Kopylova, E.; Morton, J. T.; Zech, X. Z.; Kightley, E. P.; Thompson, L. R.; Hyde, E. R.; Gonzalez, A.; et al. Deblur Rapidly Resolves Single-Nucleotide Community Sequence Patterns. mSystems 2017, 2, e00191-16. [CrossRef]
32. Price, M. N.; Dehal, P. S.; Arkin, A. P. FastTree 2-Approximately maximum-likelihood trees for large alignments. PLOS ONE 2010, 5, e9490. [CrossRef] [PubMed]
33. McDonald, D.; Price, M. N.; Goodrich, J.; Nawrocki, E. P.; DeSantis, T. Z.; Probst, A.; Andersen, G. L.; Knight, R.; Hugenholtz, P. An improved Greengenes taxonomy with explicit ranks for ecological and evolutionary analyses of bacteria and archaea. ISME J. 2012, 6, 610-618. [CrossRef] [PubMed]
34. Lozupone, C.; Lladser, M. E.; Knights, D.; Stombaugh, J.; Knight, R. UniFrac: An effective distance metric for microbial community comparison. ISME J. 2011, 5, 169-172. [CrossRef] [PubMed]
35. Benjamini, Y.; Hochberg, Y. Controlling the false discovery rate: A practical and powerful approach to multiple testing. J. R. Stat. Soc. Ser. B 1995, 57, 289-300. [CrossRef]
36. Adhikari, A.; Copping, N. A.; Onaga, B.; Pride, M. C.; Coulson, R. L.; Yang, M.; Yasui, D. H.; LaSalle, J. M.; Silverman, J. L. Cognitive deficits in the Snord116 deletion mouse model for Prader-Willi syndrome. Neurobiol. Learn. Mem. 2019, 165, 106874. [CrossRef]
37. Zieba, J.; Low, J. K.; Purtell, L.; Qi, Y.; Campbell, L.; Herzog, H.; Karl, T. Behavioural characteristics of the Prader-Willi syndrome related biallelic Snord116 mouse model. Neuropeptides 2015, 53, 71-77. [CrossRef]
38. Pinkoski, C.; Chilibeck, P. D.; Candow, D. G.; Esliger, D.; Ewaschuk, J. B.; Facci, M.; Farthing, J. P.; Zello, G. A. The effects of conjugated linoleic acid supplementation during resistance training. Med. Sci. Sports Exerc. 2006, 38, 339-348. [CrossRef]
39. Poirier, H.; Niot, I.; Clement, L.; Guerre-Millo, M.; Besnard, P. Development of conjugated linoleic acid (CLA)-mediated lipoatrophic syndrome in the mouse. Biochimie 2005, 87, 73-79. [CrossRef]
40. Jing, E.; Nillni, E. A.; Sanchez, V. C.; Stuart, R. C.; Good, D. J. Deletion of the Nhlh2 transcription factor decreases the levels of the anorexigenic peptides alpha melanocyte-stimulating hormone and thyrotropin-releasing hormone and implicates prohormone convertases I and II in obesity. Endocrinology 2004, 145, 1503-1513. [CrossRef]
41. Qi, Y.; Purtell, L.; Fu, M.; Zhang, L.; Zolotukhin, S.; Campbell, L.; Herzog, H. Hypothalamus Specific Re-Introduction of SNORD116 into Otherwise Snord116 Deficient Mice Increased Energy Expenditure. J. Neuroendocrinol. 2017, 29, el2457. [CrossRef]
42. Kruger, M.; Ruschke, K.; Braun, T. NSCL-1 and NSCL-2 synergistically determine the fate of GnRH-1 neurons and control necdin gene expression. EMBO J. 2004, 23, 4353-4364. [CrossRef] [PubMed]
43. Butler, M. G.; Miller, J. L.; Forster, J. L. Prader-Willi Syndrome-Clinical Genetics, Diagnosis and Treatment Approaches: An Update. Curr. Pediatr. Rev. 2019, 15, 207-244. [CrossRef] [PubMed]
44. Zahova, S.; Isles, A. R. Animal models for Prader-Willi syndrome. Handb. Clin. Neurol. 2021, 181, 391-404. [CrossRef]
45. Baldini, L.; Robert, A.; Charpentier, B.; Labialle, S. Phylogenetic and Molecular Analyses Identify SNORD116 Targets Involved in the Prader-Willi Syndrome. Mol. Biol. Evol. 2022, 39, msab348. [CrossRef] [PubMed]
46. Li, H.; Zhuang, P.; Zhang, Y.; Shou, Q.; Lu, Y.; Wang, G.; Qiu, J.; Wang, J.; He, L.; Chen, J.; et al. Mixed conjugated linoleic acid sex-dependently reverses high-fat diet-induced insulin resistance via the gut-adipose axis. FASEB J. 2021, 35, e21466. [CrossRef]
47. Fleck, A. K.; Hucke, S.; Teipel, F.; Eschborn, M.; Janoschka, C.; Liebmann, M.; Wami, H.; Korn, L.; Pickert, G.; Hartwig, M.; et al. Dietary conjugated linoleic acid links reduced intestinal inflammation to amelioration of CNS autoimmunity. Brain 2021, 144, 1152-1166. [CrossRef]
48. Crovesy, L.; Masterson, D.; Rosado, E. L. Profile of the gut microbiota of adults with obesity; A systematic review. Eur. J. Clin. Nutr. 2020, 74, 1251-1262. [CrossRef]
49. Ze, X.; Duncan, S. H.; Louis, P.; Flint, H. J. Ruminococcus bromii is a keystone species for the degradation of resistant starch in the human colon. ISME J. 2012, 6, 1535-1543. [CrossRef]
50. Bosshard, P. P.; Zbinden, R.; Altwegg, M. Turicibacter sanguinis gen. nov., sp. nov., a novel anaerobic, Gram-positive bacterium. Int. J. Syst. Evol. Microbiol. 2002, 52, 1263-1266. [CrossRef]
51. Coupaye, M.; Tauber, M.; Cuisset, L.; Laurier, V.; Bieth, E.; Lacorte, J. M.; Oppert, J. M.; Clement, K.; Poitou, C. Effect of Genotype and Previous GH Treatment on Adiposity in Adults With Prader-Willi Syndrome. J. Clin. Endocrinol. Metab. 2016, 101, 4895-4903. [CrossRef]
52. Bekx, M. T.; Carrel, A. L.; Shriver, T. C.; Li, Z.; Allen, D. B. Decreased energy expenditure is caused by abnormal body composition in infants with Prader-Willi Syndrome. J. Pediatr. 2003, 143, 372-376. [CrossRef]
53. Morales, J. S.; Valenzuela, P. L.; Pareja-Galeano, H.; Rincon-Castanedo, C.; Rubin, D. A.; Lucia, A. Physical exercise and Prader-Willi syndrome: A systematic review. Clin. Endocrinol. 2019, 90, 649-661. [CrossRef]
54. DeLany, J. P.; Blohm, F.; Truett, A. A.; Scimeca, J. A.; West, D. B. Conjugated linoleic acid rapidly reduces body fat content in mice without affecting energy intake. Am. J. Physiol. 1999, 276, R1172-R1179. [CrossRef]
55. Kamphuis, M. M.; Lejeune, M. P.; Saris, W. H.; Westerterp-Plantenga, M. S. The effect of conjugated linoleic acid supplementation after weight loss on body weight regain, body composition, and resting metabolic rate in overweight subjects. Int. J. Obes. Relat. Metab. Disord. 2003, 27, 840-847. [CrossRef]
56. Pariza, M. W.; Park, Y.; Cook, M. E. Conjugated linoleic acid and the control of cancer and obesity. Toxicol. Sci. 1999, 52, 107-110. [CrossRef] [PubMed]
57. Bellicha, A.; Coupaye, M.; Mosbah, H.; Tauber, M.; Oppert, J. M.; Poitou, C. Physical Activity in Patients with Prader-Willi Syndrome-A Systematic Review of Observational and Interventional Studies. J. Clin. Med. 2021, 10, 2528. [CrossRef] [PubMed]
58. Reus, L.; Zwarts, M.; van Vlimmeren, L. A.; Willemsen, M. A.; Otten, B. J.; Nijhuis-van der Sanden, M. W. Motor problems in Prader-Willi syndrome: A systematic review on body composition and neuromuscular functioning. Neurosci. Biobehav. Rev. 2011, 35, 956-969. [CrossRef] [PubMed]
59. Pamukoff, D. N.; Holmes, S. C.; Shumski, E. J.; Garcia, S. A.; Rubin, D. A. Plantar Flexor Function in Adults with and without Prader-Willi Syndrome. Med. Sci. Sports Exerc. 2020, 52, 2189-2197. [CrossRef]
60. Capodaglio, P.; Vismara, L.; Menegoni, F.; Baccalaro, G.; Galli, M.; Grugni, G. Strength characterization of knee flexor and extensor muscles in Prader-Willi and obese patients. BMC Musculoskelet Disord. 2009, 10, 47. [CrossRef]
61. Lee, S. R.; Khamoui, A. V.; Jo, E.; Zourdos, M. C.; Panton, L. B.; Ormsbee, M. J.; Kim, J. S. Effect of conjugated linoleic acids and omega-3 fatty acids with or without resistance training on muscle mass in high-fat diet-fed middle-aged mice. Exp. Physiol. 2017, 102, 1500-1512. [CrossRef]
62. Shen, W.; Chuang, C. C.; Martinez, K.; Reid, T.; Brown, J. M.; Xi, L.; Hixson, L.; Hopkins, R.; Starnes, J.; McIntosh, M. Conjugated linoleic acid reduces adiposity and increases markers of browning and inflammation in white adipose tissue of mice. J. Lipid Res. 2013, 54, 909-922. [CrossRef] [PubMed]
63. Shen, W.; McIntosh, M. K. Nutrient Regulation: Conjugated Linoleic Acid's Inflammatory and Browning Properties in Adipose Tissue. Annu. Rev. Nutr. 2016, 36, 183-210. [CrossRef] [PubMed]
64. West, D. B.; Delany, J. P.; Camet, P. M.; Blohm, F.; Truett, A. A.; Scimeca, J. Effects of conjugated linoleic acid on body fat and energy metabolism in the mouse. Am. J. Physiol. 1998, 275, R667-R672. [CrossRef] [PubMed]
65. Vyas, D.; Kadegowda, A. K.; Erdman, R. A. Dietary conjugated linoleic Acid and hepatic steatosis: Species-specific effects on liver and adipose lipid metabolism and gene expression. J. Nutr. Metab. 2012, 2012, 932928. [CrossRef] [PubMed]
66. Della Casa, L.; Rossi, E.; Romanelli, C.; Gibellini, L.; Iannone, A. Effect of diets supplemented with different conjugated linoleic acid (CLA) isomers on protein expression in C57/BL6 mice. Genes Nutr. 2016, 11, 26. [CrossRef]
67. Xiao, L.; Wang, Y.; Zhou, Y.; Sun, Y.; Sun, W.; Wang, L.; Zhou, C.; Zhou, J.; Zhang, J. Identification of a novel human cancer/testis gene MAEL that is regulated by DNA methylation. Mol. Biol. Rep. 2010, 37, 2355-2360. [CrossRef]
68. Crespo-Yanez, X.; Aguilar-Gurrieri, C.; Jacomin, A. C.; Journet, A.; Mortier, M.; Taillebourg, E.; Soleilhac, E.; Weissenhorn, W.; Fauvarque, M. O. CHMPIB is a target of USP8/UBPY regulated by ubiquitin during endocytosis. PLOS Genet. 2018, 14, e1007456. [CrossRef]
69. Mccullough, J.; Clippinger, A. K.; Talledge, N.; Skowyra, M. L.; Saunders, M. G.; Naismith, T. V.; Colf, L. A.; Afonine, P.; Arthur, C.; Sundquist, W. I.; et al. Structure and membrane remodeling activity of ESCRT-III helical polymers. Science 2015, 350, 1548-1551. [CrossRef]
70. Schmidt, O.; Teis, D. The ESCRT machinery. Curr. Biol. 2012, 22, R116-R120. [CrossRef]
71. Chang, C. L.; Weigel, A. V.; Ioannou, M. S.; Pasolli, H. A.; Xu, C. S.; Peale, D. R.; Shtengel, G.; Freeman, M.; Hess, H. F.; Blackstone, C.; et al. Spastin tethers lipid droplets to peroxisomes and directs fatty acid trafficking through ESCRT-III. J. Cell Biol. 2019, 218, 2583-2599. [CrossRef]
72. Liu, Y.; Hu, W.; Murakawa, Y.; Yin, J.; Wang, G.; Landthaler, M.; Yan, J. Cold-induced RNA-binding proteins regulate circadian gene expression by controlling alternative polyadenylation. Sci. Rep. 2013, 3, 2054. [CrossRef] [PubMed]
73. Cataldi, M.; Arnaldi, D.; Tucci, V.; De Carli, F.; Patti, G.; Napoli, F.; Pace, M.; Maghnie, M.; Nobili, L. Sleep disorders in Prader-Willi syndrome, evidence from animal models and humans. Sleep Med. Rev. 2021, 57, 101432. [CrossRef]
74. Turroni, F.; Marchesi, J. R.; Foroni, E.; Gueimonde, M.; Shanahan, F.; Margolles, A.; van Sinderen, D.; Ventura, M. Microbiomic analysis of the bifidobacterial population in the human distal gut. ISME J. 2009, 3, 745-751. [CrossRef] [PubMed]
75. Kim, E. R.; Xu, Y.; Cassidy, R. M.; Lu, Y.; Yang, Y.; Tian, J.; Li, D. P.; Van Drunen, R.; Ribas-Latre, A.; Cai, Z. L.; et al. Paraventricular hypothalamus mediates diurnal rhythm of metabolism. Nat. Commun. 2020, 11, 3794. [CrossRef]
76. Walton, J. C.; McNeill, J. K. T.; Oliver, K. A.; Albers, H. E. Temporal Regulation of GABAA Receptor Subunit Expression: Role in Synaptic and Extrasynaptic Communication in the Suprachiasmatic Nucleus. eNeuro 2017, 4. [CrossRef]
77. Rice, L. J.; Lagopoulos, J.; Brammer, M.; Einfeld, S. L. Reduced gamma-aminobutyric acid is associated with emotional and behavioral problems in Prader-Willi syndrome. Am. J. Med. Genet. B Neuropsychiatr. Genet. 2016, 171, 1041-1048. [CrossRef][PubMed]
78. Egawa, K.; Saitoh, S.; Asahina, N.; Shiraishi, H. Variance in the pathophysiological impact of the hemizygosity of gamma-aminobutyric acid type A receptor subunit genes between Prader-Willi syndrome and Angelman syndrome. Brain Dev. 2021, 43, 521-527. [CrossRef] [PubMed]
79. Ding, F.; Li, H. H.; Li, J.; Myers, R. M.; Francke, U. Neonatal maternal deprivation response and developmental changes in gene expression revealed by hypothalamic gene expression profiling in mice. PLOS ONE 2010, 5, e9402. [CrossRef]
80. Khor, E. C.; Fanshawe, B.; Qi, Y.; Zolotukhin, S.; Kulkarni, R. N.; Enriquez, R. F.; Purtell, L.; Lee, N. J.; Wee, N. K.; Croucher, P. I.; et al. Prader-Willi Critical Region, a Non-Translated, Imprinted Central Regulator of Bone Mass: Possible Role in Skeletal Abnormalities in Prader-Willi Syndrome. PLOS ONE 2016, 11, e0148155. [CrossRef]
81. Zahova, S. K.; Humby, T.; Davies, J. R.; Morgan, J. E.; Isles, A. R. Comparison of mouse models reveals a molecular distinction between psychotic illness in PWS and schizophrenia. Transl. Psychiatry 2021, 11, 433. [CrossRef]
82. Peng, Y.; Tan, Q.; Afhami, S.; Deehan, E. C.; Liang, S.; Gantz, M.; Triador, L.; Madsen, K. L.; Walter, J.; Tun, H. M.; et al. The Gut Microbiota Profile in Children with Prader-Willi Syndrome. Genes 2020, 11, 904. [CrossRef] [PubMed]
83. Zhang, C.; Yin, A.; Li, H.; Wang, R.; Wu, G.; Shen, J.; Zhang, M.; Wang, L.; Hou, Y.; Ouyang, H.; et al. Dietary Modulation of Gut Microbiota Contributes to Alleviation of Both Genetic and Simple Obesity in Children. EBioMedicine 2015, 2, 968-984. [CrossRef][PubMed]
84. Dahl, W. J.; Auger, J.; Alyousif, Z.; Miller, J. L.; Tompkins, T. A. Adults with Prader-Willi syndrome exhibit a unique microbiota profile. BMC Res. Notes 2021, 14, 51. [CrossRef] [PubMed]
85. Biassoni, R.; Di Marco, E.; Squillario, M.; Barla, A.; Piccolo, G.; Ugolotti, E.; Gatti, C.; Minuto, N.; Patti, G.; Maghnie, M.; et al. Gut Microbiota in TIDM-Onset Pediatric Patients: Machine-Learning Algorithms to Classify Microorganisms as Disease Linked. J. Clin. Endocrinol. Metab. 2020, 105, e3114-e3126. [CrossRef] [PubMed]
86. Turnbaugh, P. J.; Ley, R. E.; Mahowald, M. A.; Magrini, V.; Mardis, E. R.; Gordon, J. I. An obesity-associated gut microbiome with increased capacity for energy harvest. Nature 2006, 444, 1027-1031. [CrossRef] [PubMed]

### Example 2—Reversal of Prader-Willi Syndrome Symptoms with Tonalin™ Conjugated Linoleic Acid Supplementation

Prader-Willi Syndrome (PWS) is a devastating human genetic condition, affecting up to 1 in 10,000 live births. Affected infants present with hypotonia and development delay. Then, hyperphagia typically begins around age two years, and results in morbid obesity unless drastic calorie limitation is initiated. Them most frequent cause of PWS is a paternally-inherited 15q deletion, minimally containing the SNORD116 locus, which codes for multiple non-coding RNAs [1-3]. The maternal allele of this region is imprinted and not expressed. There is, as yet, no cure.

Many of the phenotypes in PWS patients are recapitulated by the Snord116^{m+/p-} mouse model, which was used in this Example to test the hypothesis that the nutraceutical compound conjugated linoleic acid (CLA) can be used to reduce body weight and body fat in PWS patients. Indeed, according to the Foundation for Prader-Willi Researc (fpwr.org), alleviating morbid obesity and uncontrolled hyperphagia in PWS patients would represent a significant improvement to their lives.

This Example can demonstrate at least that the Snord116^{m+/p-} mouse model shows progressive weight loss and body fat loss, without loss of muscle mass when given CLA supplementation at levels equivalent to about 0.5%/gram food. Thus, deletion of SNIRD116 does not interfere with the mechanism of action of CLA, making CLA treatment a candidate to reduce morbid obesity in these patients. This Example can at least demonstrate that improved balance and locomotion is demonstrated as a result of CLA treatment, using both the Snord116^{m+/p-} mouse model, and a mouse model containing deletion of a downstream gene, Nhlh2, as measured by a rotarod apparatus. As PWS patients have altered gait and motor performance [4,5], CLA has the potential to improve this phenotype in both patients and other individuals with balance, motor or gait problems. The use of CLA as a potential treatment for balance, and motor performance is a new finding. This Example can also demonstrate at least a reduction in anxiety, as measured by an elevated plus apparatus is shown for mice containing a deletion of Nhlh2, a gene downstream, but implicated in PWS phenotypes. There was a trend towards reduced anxiety in PWS mutant mice. In a 2020 report, over 50% of PWS patients are diagnosed with a neurobehavioral phenotypes, and those, the most common is anxiety [6].

### Results

### Reduction of Body Weight and Body Fat.

Normal an d Snord116^{m+/p-} mice (PWS model), as well as mice with a complete deletion of Snord116 (PWS KO) were randomly divided into control or CLA-treatment groups. the diets (from Envigo/Harland-Teklad) were composed of 20% fat, and matched in all other components. Conjugated linoleic acid (Tonalin™, composed of 60%-90% of the cis-9, trans-11 and trans-10, cis-12 CLA isomers in a 50/50 ratio) was provided to the CLA-treatment groups to equal 0.5% per gram food intake. This amount was calculated such that it was similar to levels found in humans necessary for weight loss ((3000 mg/day). This level of CLA also matched prior work that shows that CLA treatment can improve body weight parameters in genetically obese mice containing a deletion of Nhlh2 [7-11].

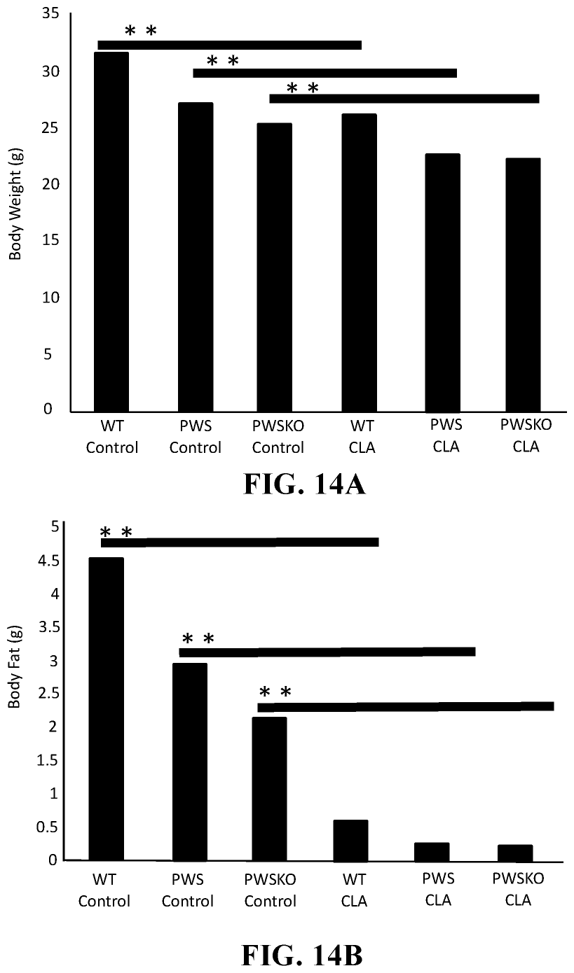

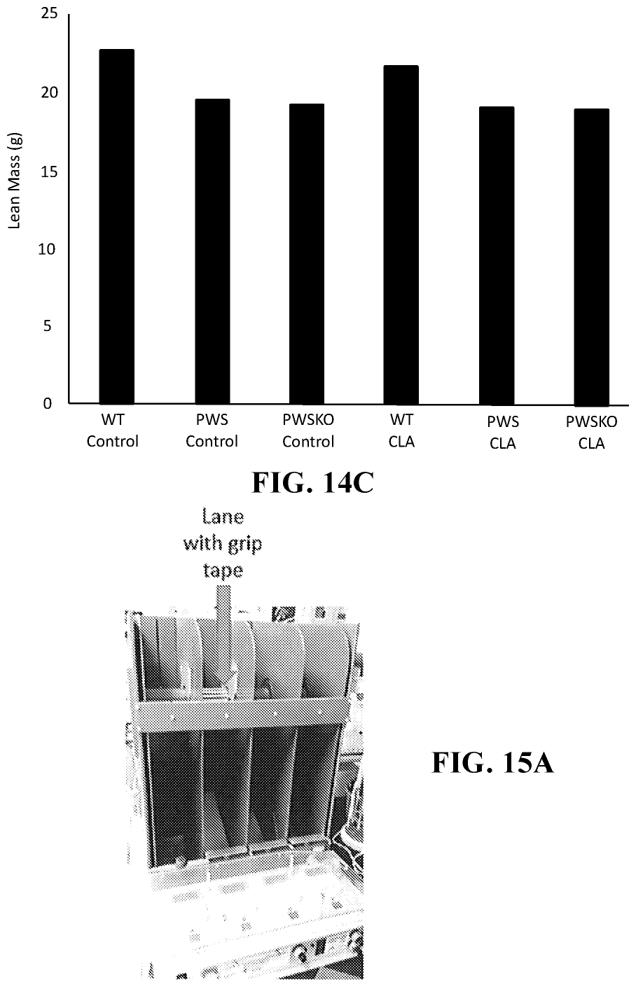

Adult mice (aged 8-10 weeks) were housed individually, and given ad libitum access to either control or CLA food for 12 weeks. They were weighed weekly, and food intake, body fat, body lean mass, and rectal temperature were measured weekly. As shown in FIG. 14A-14C, at 12 weeks, mice treated with CLA had a significant reduction in body weight, and body fat, but no changed in lean (muscle) mass.

### Improvement in Balance and Forward Locomotion.

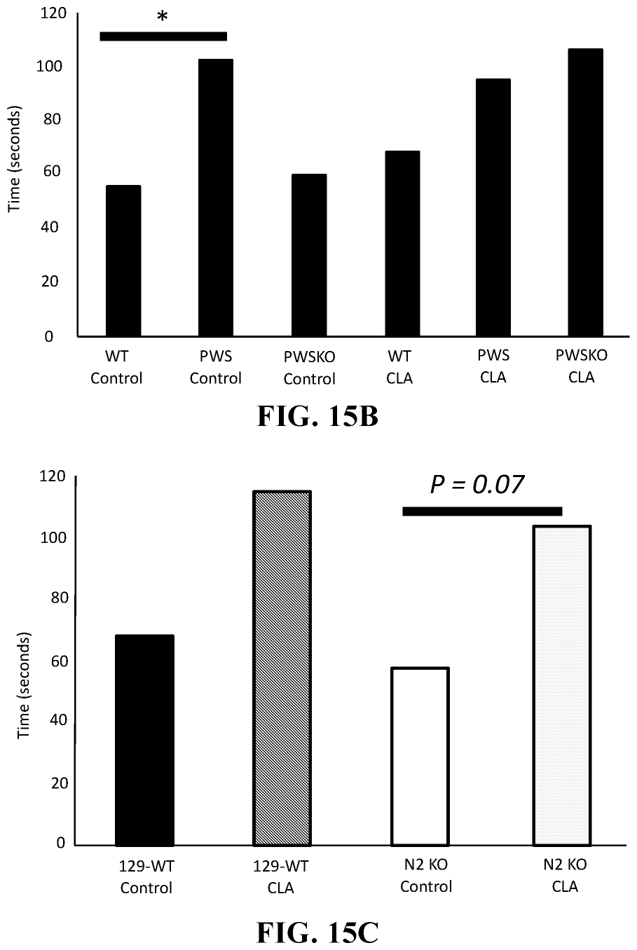

At the end of the 12 week study, mice were acclimated to using a rotarod apparatus (FIG. 15A), for one day with grip tape on the rod, and two days without grip tape, at an acceleration of 2.65%, starting from 0) rpm to a maximum speed of 20 rpm. The average of four trials, spaced at least 5 minutes apart on day 4 of testing were the basis for the experimental result. AS shown in FIG. 15B-15C, mice treated with CLA supplementation, regardless of genotype, showed balance and forward locomotion improvement.

### Improvement in Anxiety Symptoms.

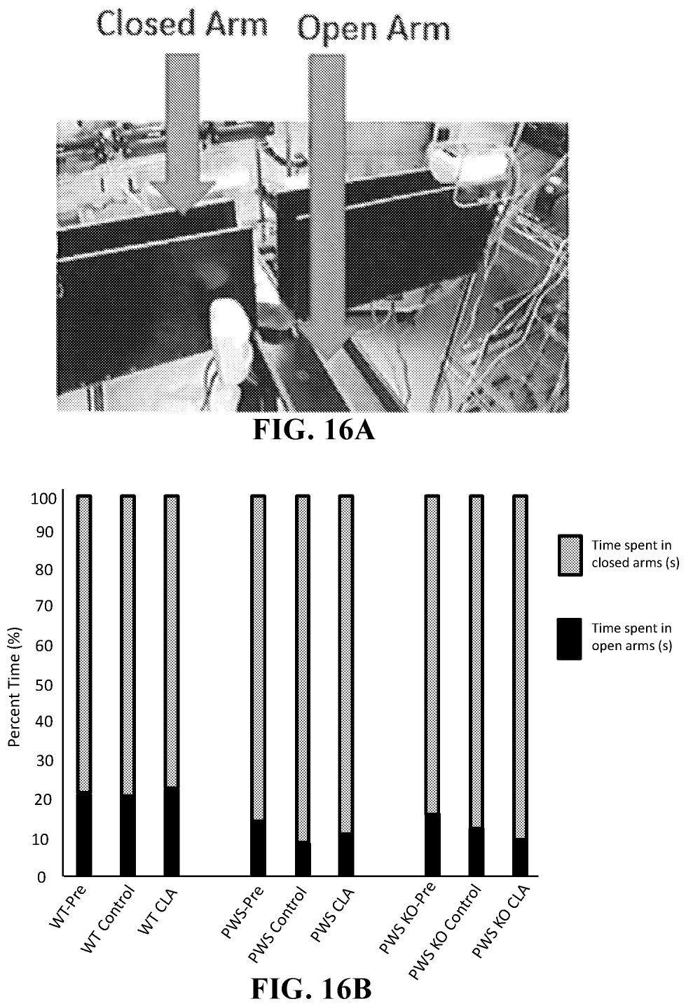

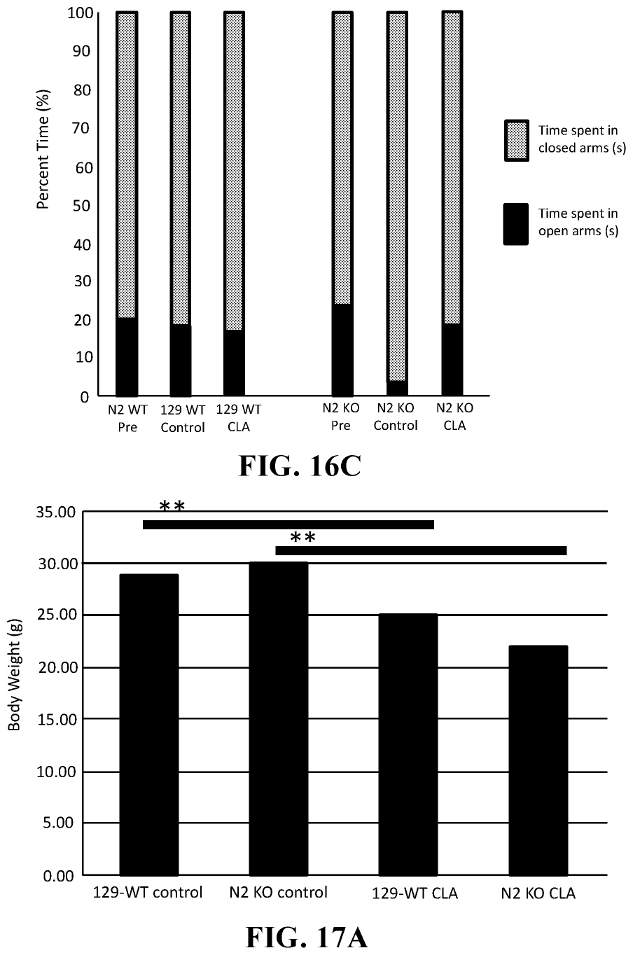

Mice were tested, using a pre-post design and an elevated plus maze for anxiety symptoms FIG. 16A. Mice that are anxious generally will spend less time in the open arms, compared to those without anxiety [12]. PWS patients show a number of neurobehavioural traits with the most commonly diagnosed phenotype being anxiety [6]. In this study mice were placed in the middle of a plus-type maze, and entrance, as well as time spent in open or closed arms over a 5 minute period were tested. Mice were tested only once before entering the 12-week feeding stud, and once at the end of the 12-week feeding study. The percent time is reported. The data shown in FIG. 16B-16C show a comparison between pre-week test results, and the two treatment groups for each genotype in the post-week test. As shown in FIGS. 16B and 16C, the percent time in open arms was less for all animals. However, PWS mice spent significantly less time in the open arms than WT mice, and there was a numeric improvement with CLA treatment (FIG. 16B). There appeared to be no difference in anxiety levels between WT and Nhlh2 KO mice in the pre-test, but a highly significant improvement for mice following 12 weeks of CLA treatment as compared to control diet treated mice (FIG. 16C). As these are two different strains of mice, the data suggest that genetic strain differences do not affect CLA action. These data suggest that CLA treatment may improve anxiety in both young and older mice. These findings can be translated to treatment of humans with anxiety.

The data in at least this Example at least supports treatment of PWS patients with CLA. CLA is categorized as generally recognized as safe (GRAS) by the Food and Drug Administration at therapeutic levels of at least 3,0000 mg/day [9]. Further these data demonstrate that the correct isomer mix of 50:50 cis-9 and trans-10, cis-12 isomers that makes up at least 80 percent of the at least 3,0000 mg/day CLA [13]. Further different dosage forms can be considered such as pills or liquid beverages, such as shakes, the latter of which may be preferable for children or patients difficult to administer pills to. The data in at least this Example supports the use of the CLA formulation for aging or elderly patients and for treatment of balance and/or locomotion disorders in patients. The data in at least this Example supports the use of the CLA formulation for treatment of anxiety in PWS patients as well as patients with non-PWS associated anxiety

### REFERENCES FOR EXAMPLE 2

1. Cavaille, J. Wiley Interdiscip Rev RNA. 2017 July; 8(4). doi: 10.1002/wrna. 1417. Epub 2017 Mar. 13.
2. Polex-Wolf et al., 2018. J Clin Inves. 128:960-969.
3. Tan et al., 2020. Genes (Basel) 11.
4. Belluscio et al., 2019. Hum Mov Sci. 63:53-61.
5. Chiu et al., 2017. Peer J. 5(e4097)
6. Feighan et al., 2020. J Intellect Disabil Res. 64:158-169.
7. Hur et al., 2009. J Med Food. 12, 56-63.
8. Kim et al., 2012. Food Funct. 3:1280-1285.
9. Kim et al., 2016. Annu Rev Food Sci Technol, 7, 221-244.
10. Kim et al., 2013. J Nutr Biochem. 24, 556-566.
11. Kim et al., 2015. J Agric Food Chem 63. 5212-5223.
12. La-Vu et al., 2020. Front Behav Neurosci. 14, 145.
13. Racine et al., 2010. Am J Clin Nutr 91:1157-1164.

### Example 3—CLA Treatment of Hypogonadism

In males, incidence of HH is as high as one in 4000, with incidence in females being about 2-5 times lower [1,2]. It was previously shown that mice containing a mutation of Nhlh2, a neuronal basic-helix-loop-helix transcription factor show a failure of pubertal onset, adult-onset obesity and hypogonadism. These mice are termed N2KO mice. In other prior studies, treatment of N2KO mice resulted in a reduction in body weight and body fat [3-6]. In this Example, N2KO and WT mice were treated with about 0.5% CLA for 12 weeks. N2KO mice showed both reduced body weight and body fat, consistent with prior data.

At the end of the study, numerous body tissues were taken during necropsy, including testes. Testes were embedded for histology, stained with H&E. Without being bound by theory, the data supports that N2KO mice on the CLA supplemented diet had improved testicular morphology as compared to N2KO mice on a control diet. There appeared to be no effect in WT mice.

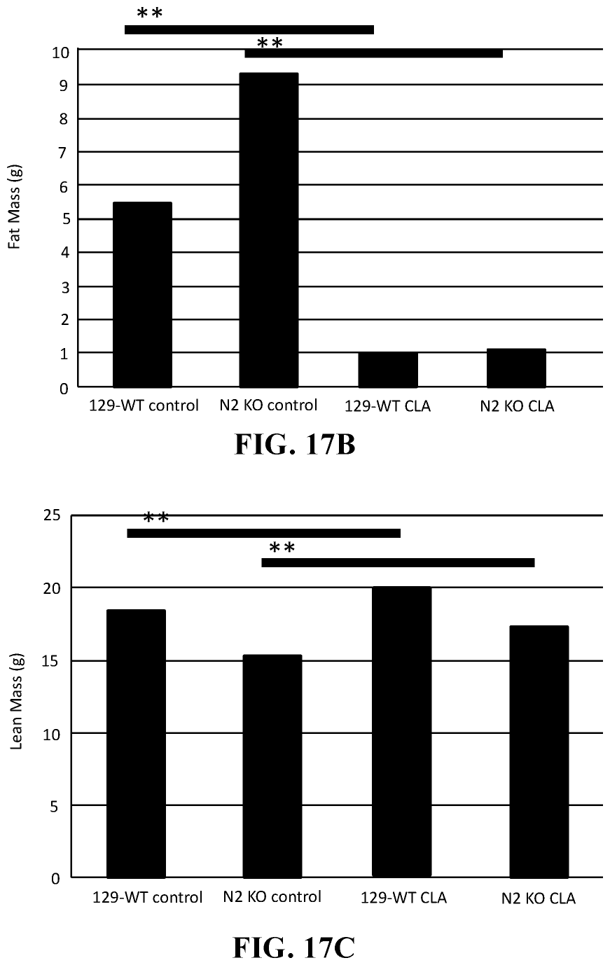

As shown in FIG. 17A-17B, N2KO mice with genetic obesity respond to CLA supplementation with improved body weight and decreased body fat (FIG. 17A-17B). Also and importantly, lean mass, which represents muscle, is improved with CLA treatment (FIG. 17C). This data is consistent with prior results.

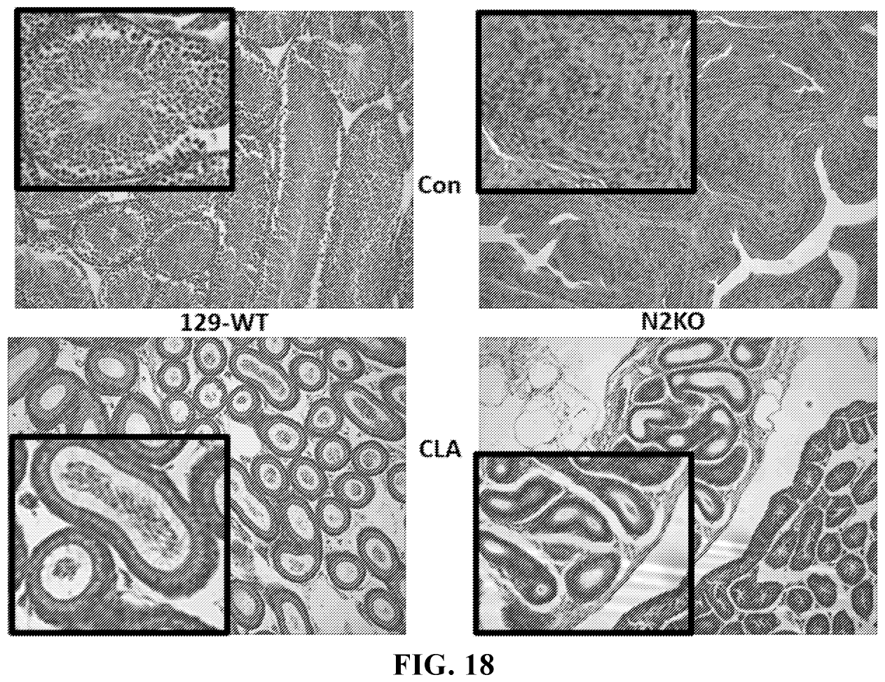

At the end of the 12-week feeding study with supplemental CLA, mice were euthanized and necropsied. Testicular tissue was removed from the abdominal cavity and placed in 4% paraformaldehyde overnight. The next day, tissue was dehydrated in 70% ethanol and stored at 4 degrees C. until further processing, which included embedding, sectioning and H&E staining. As shown in FIG. 18, normal mice have normal testicular morphology following 12 weeks of both control and CLA supplemented diet. However, N2KO mice on control diet displayed the typical degeneration of the testes, seen for this mouse model. Surprisingly, N2KO mice fed the CLA diet have an improved testicular morphology. Rather than degeneration, testicular tubules are seen, with the apparent presence of sperm in the epididymis. Testis size was not influenced by diet.

This data supports at least the use of CLA, such as Tonalin™ CLA for treatment of hypogonadism, improve testicular morphology, fertility and virility, in patients, particularly human patients. This data supports the use of CLA to simultaneously treated hypogonadism and excess fat/obesity in subjects, particularly human subjects.

### REFERENCES FOR EXAMPLE 3

1. Silveira and Latronico. 2013. J Clin Endocrinol. Metab. 98:1781-1788.
2. Good, D. J. 2021. Mol Cell Endocrinol. 520:111077.
3. Hur et al., 2009. J Med Food. 12:56-63.
4. Kim et al., 2012. Food Funct. 3:1280-1285.
5. Kim et al., 2013. J Nutr Biochem 24:556-566.
6. Kim et al., 2015. J Agric Food Chem. 63:5212-5223.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

Further attributes, features, and embodiments of the present invention can be understood by reference to the following numbered aspects of the disclosed invention. Reference to disclosure in any of the preceding aspects is applicable to any preceding numbered aspect and to any combination of any number of preceding aspects, as recognized by appropriate antecedent disclosure in any combination of preceding aspects that can be made. The following numbered aspects are provided:
1. A method of treating a Snord116 deficiency, disease, or a symptom thereof in a subject in need thereof, the method comprising:
   administering an effective amount of conjugated linoleic acid (CLA) or a formulation thereof to the subject in need thereof, optionally wherein the effective amount is administered in one or more doses.
2. The method of aspect 1, wherein the Snord116 deficiency disease is Prader-Willi Syndrome (PWS).
3. The method of any one of aspects 1-2, wherein the effective amount of CLA is at least 1,000 mg per day, optionally about 3,000 to about 5,000 mg per day.
4. The method of any one of aspects 1-3, wherein the effective amount of the CLA is about 3-10 g/kg body weight per day, optionally about 5 g/kg body weight per day.
5. The method of any one of aspects 1-4, wherein the CLA or formulation thereof comprises a mixture of CLA isoforms, optionally a 50:50 mixture of trans-10, cis-12 and trans9, cis-11 CLA isomers.
6. The method of any one of aspects 1-5, wherein the CLA or formulation thereof comprises 80-100% of a mixture of CLA isoforms, optionally a 50:50 mixture of trans-10, cis-12 and trans9, cis-11 CLA isomers.
7. The method of any one of aspects 1-6, wherein the effective amount of CLA decreases body weight, decreases fat mass, increases lean body mass, improves hypogonadism or a symptom thereof, improves testicular morphology, modulates the α-diversity of the gut microbiome, modulates hypothalamic gene expression of one or more genes, decreases anxiety, increase sperm maturation, increase spermatogenesis, increase sperm differentiation, decrease testicular degeneration or any combination thereof in the subject in need thereof.
8. The method of any one of aspects 1-7, wherein the effective amount of CLA or formulation thereof is administered daily, every other day, weekly, or monthly.
9. The method of any one of aspects 1-8, wherein the effective amount of CLA or formulation thereof is a liquid, solid, semi-solid, or an emulsion.
10. The method of any one of aspects 1-9, wherein the effective amount of CLA or formulation thereof is a dietary supplement or feed or foodstuff or beverage formulation.
11. The method of any one of aspects 1-10, wherein the subject in need thereof is a mammal, optionally a human.
12. A dietary supplement, feed, or foodstuff or beverage formulation effective for treating a Snord116 deficiency disease or a symptom thereof in a subject in need thereof, the dietary supplement comprising:
   an amount of conjugated linoleic acid (CLA) or a formulation thereof such that the dietary supplement delivers an effective amount of the CLA to the subject in need thereof in one or more doses, optionally 1-3 doses.
13. The dietary supplement, feed, or foodstuff or beverage formulation of aspect 12, wherein the Snord116 deficiency disease is Prader-Willi Syndrome (PWS).
14. The dietary supplement, feed, or foodstuff or beverage formulation of any one of aspects 12-13, wherein the effective amount of CLA is at least 1,000 mg per day, optionally about 3,000 to about 5,000 mg per day.
15. The dietary supplement, feed, or foodstuff or beverage formulation of any one of aspects 12-14, wherein the effective amount of the CLA is about 3-10 g/kg body weight per day, optionally about 5 g/kg body weight per day.
16. The dietary supplement, feed, or foodstuff or beverage formulation of any one of aspects 12-15, wherein the CLA or formulation thereof comprises a mixture of CLA isoforms, optionally a 50:50 mixture of trans-10, cis-12 and trans9, cis-11 CLA isomers.
17. The dietary supplement, feed, or foodstuff or beverage formulation of any one of aspects 12-16, wherein the CLA or formulation thereof comprises 80-100% of a mixture of CLA isoforms, optionally a 50:50 mixture of trans-10, cis-12 and trans9, cis-11 CLA isomers.
18. The dietary supplement, feed, or foodstuff or beverage formulation of any one of aspects 12-17, wherein the effective amount of CLA decreases body weight, decreases fat mass, increases lean body mass, improves hypogonadism or a symptom thereof, improves testicular morphology, modulates the α-diversity of the gut microbiome, modulates hypothalamic gene expression of one or more genes, decreases anxiety, increase sperm maturation, increase spermatogenesis, increase sperm differentiation, decrease testicular degeneration or any combination thereof in the subject in need thereof.
19. The dietary supplement, feed, or foodstuff or beverage formulation of any one of aspects 12-18, wherein the dietary supplement, feed, or foodstuff or beverage formulation is a liquid, solid, semi-solid, or an emulsion.
20. The dietary supplement, feed, or foodstuff or beverage formulation of any one of aspects 12-19, wherein the dietary supplement, feed, or foodstuff or beverage formulation is adapted for daily, every other day, weekly, or monthly administration.
21. The dietary supplement, feed, or foodstuff or beverage formulation of any one of aspects 12-20, wherein the subject in need thereof is a mammal, optionally a human.
22. A method of treating hypogonadism in a subject having a Nhlh2 deficiency, disease, or symptom thereof, the method comprising:
   an effective amount of conjugated linoleic acid (CLA) or a formulation thereof to the subject in need thereof.
23. The method of aspect 22, wherein the effective amount of CLA is at least 1,000 mg per day, optionally about 3,000 to about 5,000 mg per day.
24. The method of any one of aspects 22-23, wherein the effective amount of the CLA is about 3-10 g/kg body weight per day, optionally about 5 g/kg body weight per day.
25. The method of any one of aspects 22-24, wherein the CLA or formulation thereof comprises a mixture of CLA isoforms, optionally a 50:50 mixture of trans-10, cis-12 and trans9, cis-11 CLA isomers.
26. The method of any one of aspects 22-25, wherein the CLA or formulation thereof comprises 80-100% of a mixture of CLA isoforms, optionally a 50:50 mixture of trans-10, cis-12 and trans9, cis-11 CLA isomers.
27. The method of any one of aspects 22-26, wherein the effective amount of CLA decreases body weight, decreases fat mass, increases lean body mass, improves hypogonadism or a symptom thereof, improves testicular morphology, increases sperm maturation, increase spermatogenesis, increase sperm differentiation, decrease testicular degeneration or any combination thereof in the subject in need thereof.
28. The method of any one of aspects 22-27, wherein the effective amount of CLA or formulation thereof is administered daily, every other day, weekly, or monthly.
29. The method of any one of aspects 22-28, wherein the effective amount of CLA or formulation thereof is a liquid, solid, semi-solid, or an emulsion.
30. The method of any one of aspects 22-29, wherein the effective amount of CLA or formulation thereof is a dietary supplement or feed or foodstuff or beverage formulation.
31. The method of any one of aspects 22-30, wherein the subject in need thereof is a mammal, optionally a human.
32. A dietary supplement, feed, or foodstuff or beverage formulation hypogonadism in a subject having a Nhlh2 deficiency, disease, or symptom thereof in a subject in need thereof, the dietary supplement comprising:
   an amount of conjugated linoleic acid (CLA) or a formulation thereof such that the dietary supplement delivers an effective amount of the CLA to the subject in need thereof in one or more doses, optionally 1-3 doses.
33. The dietary supplement, feed, or foodstuff or beverage formulation of aspect 32, wherein the effective amount of CLA is at least 1,000 mg per day, optionally about 3,000 to about 5,000 mg per day.
34. The dietary supplement, feed, or foodstuff or beverage formulation of any one of aspects 32-33, wherein the effective amount of the CLA is about 3-10 g/kg body weight per day, optionally about 5 g/kg body weight per day.
35. The dietary supplement, feed, or foodstuff or beverage formulation of any one of aspects 32-34, wherein the CLA or formulation thereof comprises a mixture of CLA isoforms, optionally a 50:50 mixture of trans-10, cis-12 and trans9, cis-11 CLA isomers.
36. The dietary supplement, feed, or foodstuff or beverage formulation of any one of aspects 32-35, wherein the CLA or formulation thereof comprises 80-100% of a mixture of CLA isoforms, optionally a 50:50 mixture of trans-10, cis-12 and trans9, cis-11 CLA isomers.
37. The dietary supplement, feed, or foodstuff or beverage formulation of any one of aspects 32-36, wherein the effective amount of CLA decreases body weight, decreases fat mass, increases lean body mass, improves hypogonadism or a symptom thereof, improves testicular morphology, increases sperm maturation, increase spermatogenesis, increase sperm differentiation, decrease testicular degeneration or any combination thereof in the subject in need thereof.
38. The dietary supplement, feed, or foodstuff or beverage formulation of any one of aspects 32-37, wherein the dietary supplement, feed, or foodstuff formulation is a liquid, solid, semi-solid, or an emulsion.
39. The dietary supplement, feed, or foodstuff or beverage formulation of any one of aspects 32-38, wherein the dietary supplement, feed, or foodstuff formulation is adapted for daily, every other day, weekly, or monthly administration.
40. The dietary supplement, feed, or foodstuff or beverage formulation of any one of aspects 32-39, wherein the subject in need thereof is a mammal, optionally a human.

What is claimed is:

1. A method of treating a Snord116 deficiency, disease, or a symptom thereof in a subject in need thereof, the method comprising:
administering an effective amount of conjugated linoleic acid (CLA) or a formulation thereof to the subject in need thereof, optionally wherein the effective amount is administered in one or more doses.

2. The method of claim 1, wherein the Snord116 deficiency disease is Prader-Willi Syndrome (PWS).

3. The method of claim 1, wherein the effective amount of CLA is at least 1,000 mg per day, optionally about 3,000 to about 5,000 mg per day.

4. The method of claim 1, wherein the effective amount of the CLA is about 3-10 g/kg body weight per day, optionally about 5 g/kg body weight per day.

5. The method of claim 1, wherein the CLA or formulation thereof comprises a mixture of CLA isoforms, optionally a 50:50 mixture of trans-10, cis-12 and trans9, cis-11 CLA isomers.

6. The method of claim 1, wherein the CLA or formulation thereof comprises 80-100% of a mixture of CLA isoforms, optionally a 50:50 mixture of trans-10, cis-12 and trans9, cis-11 CLA isomers.

7. The method of claim 1, wherein the effective amount of CLA decreases body weight, decreases fat mass, increases lean body mass, improves hypogonadism or a symptom thereof, improves testicular morphology, modulates the α-diversity of the gut microbiome, modulates hypothalamic gene expression of one or more genes, decreases anxiety, increase sperm maturation, increase spermatogenesis, increase sperm differentiation, decrease testicular degeneration or any combination thereof in the subject in need thereof.

8. The method of claim 1, wherein the effective amount of CLA or formulation thereof is administered daily, every other day, weekly, or monthly.

9. The method of claim 1, wherein the effective amount of CLA or formulation thereof is a liquid, solid, semi-solid, or an emulsion.

10. The method of claim 1, wherein the effective amount of CLA or formulation thereof is a dietary supplement or feed or f foodstuff or beverage formulation.

11. The method of claim 1, wherein the subject in need thereof is a mammal, optionally a human.

12. A dietary supplement, feed, or foodstuff or beverage formulation effective for treating a Snord116 deficiency disease or a symptom thereof in a subject in need thereof, the dietary supplement comprising:
an amount of conjugated linoleic acid (CLA) or a formulation thereof such that the dietary supplement delivers an effective amount of the CLA to the subject in need thereof in one or more doses, optionally 1-3 doses.

13. The dietary supplement, feed, or foodstuff or beverage formulation of claim 12, wherein the Snord116 deficiency disease is Prader-Willi Syndrome (PWS).

14. The dietary supplement, feed, or foodstuff or beverage formulation of claim 12, wherein the effective amount of CLA is at least 1,000 mg per day, optionally about 3,000 to about 5,000 mg per day.

15. The dietary supplement, feed, or foodstuff or beverage formulation of claim 12, wherein the effective amount of the CLA is about 3-10 g/kg body weight per day, optionally about 5 g/kg body weight per day.

16. The dietary supplement, feed, or foodstuff or beverage formulation of claim 12, wherein the CLA or formulation thereof comprises a mixture of CLA isoforms, optionally a 50:50 mixture of trans-10, cis-12 and trans9, cis-11 CLA isomers.

17. The dietary supplement, feed, or foodstuff or beverage formulation of claim 12, wherein the CLA or formulation thereof comprises 80-100% of a mixture of CLA isoforms, optionally a 50:50 mixture of trans-10, cis-12 and trans9, cis-11 CLA isomers.

18. The dietary supplement, feed, or foodstuff or beverage formulation of claim 12, wherein the effective amount of CLA decreases body weight, decreases fat mass, increases lean body mass, improves hypogonadism or a symptom thereof, improves testicular morphology, modulates the α-diversity of the gut microbiome, modulates hypothalamic gene expression of one or more genes, decreases anxiety, increase sperm maturation, increase spermatogenesis, increase sperm differentiation, decrease testicular degeneration or any combination thereof in the subject in need thereof.

19. The dietary supplement, feed, or foodstuff or beverage formulation of claim 12, wherein the dietary supplement, feed, or foodstuff or beverage formulation is a liquid, solid, semi-solid, or an emulsion.

20. The dietary supplement, feed, or foodstuff or beverage formulation of claim 12, wherein the dietary supplement, feed, or foodstuff or beverage formulation is adapted for daily, every other day, weekly, or monthly administration.

21. The dietary supplement, feed, or foodstuff or beverage formulation of claim 12, wherein the subject in need thereof is a mammal, optionally a human.

22. A method of treating hypogonadism in a subject having a Nhlh2 deficiency, disease, or symptom thereof, the method comprising:
an effective amount of conjugated linoleic acid (CLA) or a formulation thereof to the subject in need thereof.

23. The method of claim 22, wherein the effective amount of CLA is at least 1,000 mg per day, optionally about 3,000 to about 5,000 mg per day.

24. The method of claim 22, wherein the effective amount of the CLA is about 3-10 g/kg body weight per day, optionally about 5 g/kg body weight per day.

25. The method of claim 22, wherein the CLA or formulation thereof comprises a mixture of CLA isoforms, optionally a 50:50 mixture of trans-10, cis-12 and trans9, cis-11 CLA isomers.

26. The method of claim 22, wherein the CLA or formulation thereof comprises 80-100% of a mixture of CLA isoforms, optionally a 50:50 mixture of trans-10, cis-12 and trans9, cis-11 CLA isomers.

27. The method of claim 22, wherein the effective amount of CLA decreases body weight, decreases fat mass, increases lean body mass, improves hypogonadism or a symptom thereof, improves testicular morphology, increases sperm maturation, increase spermatogenesis, increase sperm differentiation, decrease testicular degeneration or any combination thereof in the subject in need thereof.

28. The method of claim 22, wherein the effective amount of CLA or formulation thereof is administered daily, every other day, weekly, or monthly.

29. The method of claim 22, wherein the effective amount of CLA or formulation thereof is a liquid, solid, semi-solid, or an emulsion.

30. The method of claim 22, wherein the effective amount of CLA or formulation thereof is a dietary supplement or feed or foodstuff or beverage formulation.

31. The method of claim 22, wherein the subject in need thereof is a mammal, optionally a human.

32. A dietary supplement, feed, or foodstuff or beverage formulation hypogonadism in a subject having a Nhlh2 deficiency, disease, or symptom thereof in a subject in need thereof, the dietary supplement comprising:
an amount of conjugated linoleic acid (CLA) or a formulation thereof such that the dietary supplement delivers an effective amount of the CLA to the subject in need thereof in one or more doses, optionally 1-3 doses.

33. The dietary supplement, feed, or foodstuff or beverage formulation of claim 32, wherein the effective amount of CLA is at least 1,000 mg per day, optionally about 3,000 to about 5,000 mg per day.

34. The dietary supplement, feed, or foodstuff or beverage formulation of claim 32, wherein the effective amount of the CLA is about 3-10 g/kg body weight per day, optionally about 5 g/kg body weight per day.

35. The dietary supplement, feed, or foodstuff or beverage formulation of claim 32, wherein the CLA or formulation thereof comprises a mixture of CLA isoforms, optionally a 50:50 mixture of trans-10, cis-12 and trans9, cis-11 CLA isomers.

36. The dietary supplement, feed, or foodstuff or beverage formulation of claim 32, wherein the CLA or formulation thereof comprises 80-100% of a mixture of CLA isoforms, optionally a 50:50 mixture of trans-10, cis-12 and trans9, cis-11 CLA isomers.

37. The dietary supplement, feed, or foodstuff or beverage formulation of claim 32, wherein the effective amount of CLA decreases body weight, decreases fat mass, increases lean body mass, improves hypogonadism or a symptom thereof, improves testicular morphology, increases sperm maturation, increase spermatogenesis, increase sperm differentiation, decrease testicular degeneration or any combination thereof in the subject in need thereof.

38. The dietary supplement, feed, or foodstuff or beverage formulation of claim 32, wherein the dietary supplement, feed, or foodstuff or beverage formulation is a liquid, solid, semi-solid, or an emulsion.

39. The dietary supplement, feed, or foodstuff or beverage formulation of claim 32, wherein the dietary supplement, feed, or foodstuff or beverage formulation is adapted for daily, every other day, weekly, or monthly administration.

40. The dietary supplement, feed, or foodstuff or beverage formulation of claim 32, wherein the subject in need thereof is a mammal, optionally a human.
